(19) United States
(12) Patent Application Publication
(10) Pub. No.: US 2019/0328753 A1
(43) Pub. Date: Oct. 31, 2019

(54) COMPOSITIONS AND METHODS FOR TREATING OCULAR DISEASES

(71) Applicants: Richard W. Yee, Houston, TX (US); Kenneth Hughes, Houston, TX (US)

(72) Inventors: Richard W. Yee, Houston, TX (US); Kenneth Hughes, Houston, TX (US)

(21) Appl. No.: 16/510,551

(22) Filed: Jul. 12, 2019

Related U.S. Application Data
(63) Continuation of application No. PCT/US19/13216, filed on Jan. 11, 2019.
Application No. PCT/US19/13216 is a continuation-in-part of application No. 15/555,527, filed on Sep. 4, 2017, now Pat. No. 10,350,223.
(60) Provisional application No. 62/616,289, filed on Jan. 11, 2018.
(60) Provisional application No. 62/127,362, filed on Mar. 3, 2015.

(51) Int. Cl. A61K 31/585; A61P 27/04

(52) U.S. Cl. CPC A61K 31/585; A61P 27/04

(57) ABSTRACT

Described methods and compositions for treating Meibomian Gland Disease (MGD) for normalizing gland secretions and improving symptoms of ocular surface diseases associated with MGD. The methods concern treatment of a patient with aldosterone antagonists, such as spironolactone or analogues of spironolactone. Spironolactone is desirably added in a novel treatment composition, preferably in the form of an aqueous solution, emulsion or suspension in small but effective concentrations and in a novel vehicle that adds to the increased solubility of what previously was known to be an insoluble active agent. Moreover, it is believed that the specific lower but effective concentrations of spironolactone, and/or the pluronic vehicle of the treatment composition, permits optimal expression of essential lipids and upregulation of genes that control lipid production necessary to a more normalized lipid component of the tear film for the treatment and/or the prevention of signs and/or symptoms of MGD.

### CROSS-REFERENCE TO RELATED APPLICATIONS

The present application is a Continuation application of International Application No. PCT/US19/13216, filed Jan. 11, 2019, which claims priority to and the benefit of the filing date of U.S. Provisional Application No. 62/616,289, filed Jan. 11, 2018, and the present application is a Continuation-in-Part Application of U.S. application Ser. No. 15/555,527, filed Sep. 4, 2017, which is a National Stage application under 35 U.S.C. § 371 of International Application No. PCT/US16/20683, filed Mar. 3, 2016, which international application claims priority to and the benefit of the filing date of U.S. Provisional Application No. 62/127,362, filed Mar. 3, 2015. The disclosure of each of these applications is hereby incorporated by reference herein in their entireties.

### SEQUENCE LISTING

The instant application contains a Sequence Listing which has been submitted electronically in ASCII format and is hereby incorporated by reference in its entirety, as well as in paper form. The information recorded in computer readable form is identical to the written (on paper) sequence listing and does not include matter which goes beyond the disclosure in International Application No. PCT/US19/13216, filed Jan. 11, 2019. Said ASCII copy, created on Jul. 12, 2019, is named YEE-101-CIP_seq_list_ST25 and is 58 KB in size.

### FIELD OF THE INVENTION

Disclosed herein are compositions for treating ocular diseases. Further disclosed are methods of using such compositions for treating ocular disorders of the eye by administering the described compositions to an ocular region or periocular region of a patient, which include the ocular surface, the eyelid glands, and the posterior segment, e.g., the retina.

### BACKGROUND OF THE INVENTION

Ocular surface diseases affect millions of Americans each year (see Schein et al., American J. Ophthalmology, 124:723-738, (1997)). One such ocular surface disease, “dry eye disease”, a generic description for an ocular surface disease of the tear film, can cause considerable pain and discomfort to those afflicted. Mild cases may only present symptoms of drying or irritation, while more severe cases may include burning sensations or substantial impairments to a person's vision.

Chronic dryness can adversely impact normal daily activities such as reading, driving, and engaging in outdoor activities among other things. Ocular surface diseases, such as dry eye, are on the rise, and particularly among older populations. It has been estimated that approximately 4.9 million Americans 50 years and older have dry eye or chronic dry eye disease with the number of women appearing to outnumber men similarly affected. (see Smith, The Ocular Surface 5(2): 93-407 (2007)).

Dry eye is typically divided into two broad classes—aqueous tear-deficient dry eye (ADDE) and evaporative dry eye (EDE). The former, ADDE, generally refers to a disorder in which the lacrimal glands fail to produce enough of the watery component of tears to maintain a healthy eye surface. ADDE can be further divided in to two major subclasses, Sjogren syndrome dry eye and non-Sjogren syndrome dry eye (primary and secondary lacrimal gland deficiencies, obstruction of the lacrimal gland ducts, reflex hyposecretion, reflex motor block). The latter, EDE, is generally characterized by excessive water loss from the ocular surface due to evaporation. As with ADDE, EDE, can also be divided into two major subclasses—intrinsically caused and extrinsically caused EDE. Intrinsic causes can include inflammation of the meibomian glands which make the lipid or oily part of tears that slows evaporation and keeps the tears stable, eye lid disorders, and infrequent blinking. EDE is often referred to as posterior blepharitis, meibomian gland disease or meibomitis. Extrinsic causes can include other ocular surface disorders, diseases, or infections, contact lens wear, and allergies.

Many ocular surface diseases, including dry eye, are characterized by the presence by a common final pathway of inflammatory related lesions during the examination of the ocular surface. Early signs of ocular surface disease, primary or secondary to inflammation, may further include redness, chemosis, and/or engorged vasculature (usually localized in bulbar or palpebral conjunctiva). Later stage ocular surface disease often manifests further due to inadequate, intermittent, and/or untreated inflammation. Signs and symptoms of mature ocular surface disease include anterior lid margin vascularization around the orifice, obstruction of varying degrees of the meibum secretion, degrees of meibum viscosity and turbidity, Zone A posterior lid margin vascularization, chalasis, and meibomian gland loss and/or drop out. These lesions can be accompanied by vascularization suggesting both acute and chronic processes that will be ongoing unless multiple treatment approaches quell the common final pathways promoting the chronic morbidity associated with the varying degrees and frequency of inflammatory insults.

A number of risk factors found to correlate to the development of dry eye have been identified and include being female, older age, postmenopausal estrogen therapy, diabetes, a diet that is low in omega 3 essential fatty acids or has a high ratio of omega 6 to omega 3 fatty acids, refractive surgery, vitamin A deficiency, radiation therapy, bone marrow transplant, hepatitis C, certain classes of systemic and ocular medications including antihistamines (see Smith, 2007). Other risk factors may include autoimmune deficiencies, microbial infection (viral and/or bacterial), connective tissue diseases, systemic cancer chemotherapy, and certain medications (see Smith, 2007).

Current solutions for treating dry eye include tear supplementation (e.g., lubricants), tear retention, tear stimulation, tear substitutes, anti-inflammatory therapy, and essentially fatty acids, and environmental strategies. (see Pflugfelder, The Ocular Surface 5(2): 163-178, 2007). Non-limiting examples of current solutions include topical artificial tears, topical cyclosporine A (commercially available as RESTASIS®), lifitegrast (commercially available as XIIDRA®), systemic omega 3 fatty acids, systemic flaxseed, oral antibiotics (i.e., minocycline, doxycycline, tetracycline, azithromycin), topical antibiotics, oral steroids, topical steroids, topical non-steroidals, topical anti-allergy drops, as well as manual procedures, including mechanical opening and clearing of blocked glands (e.g., meibomian gland probing and LIPIFLOW®), intense pulse light therapy, punctal plugs, and punctal cautery. Of the solutions discussed, lubricants are the easiest, least invasive, and most frequently employed solution to dry eye. The effects of lubricants, however, are ephemeral and require constant reapplication for sustained relief. None of the current solutions are sufficient—a broader spectrum solution is needed to alleviate the symptoms associated with dry eye disease.

The present disclosure relates to the topical treatment of ocular surface diseases, particularly dry eye, with topical applications of at least one aldosterone antagonist in the eye or surrounding adnexal structures surrounding or adjacent to the ocular surface (including the tear film, cornea, conjunctiva including goblet cells, ocular lymphatics, eye lids, eye lid glands including for example lacrimal glands, accessory lacrimal glands, such as glands of Zeiss and Moll, meibomian glands (such as meibomian gland ducts and peri-ductal regions, including associated ductal cells, and peri-ductal cells, such as acinar cells), glands of Wolfring, etc.), as a way of providing therapeutically useful concentrations of the drug at its site of action. This class of topical drugs with its diuretic, anti-inflammatory, antiandrogenic, lipid producing capabilities, and other unspecified drug actions on the ocular surface may be therapeutic locally while minimizing drug entry into the blood stream, and therefore, preventing or avoiding possible systemic side effects. For example, in vitro data shows that aldosterone antagonists such as spironolactone are capable of inducing lipid like secretions of corneal epithelial cells and possibly other cells of the ocular surface and associated lid adnexa, including the meibomian glands of the eyelid and its cellular components. The topical or injectable applications can include other active agents such as anti-inflammatories and/or antibiotics, including dapsone (diaminodiphenyl sulfone or DDS), which provides both anti-inflammatory effects and antibiotic effects.

Aldosterone antagonists have been used in the cosmetic and skin care industries. For example, the aldosterone antagonist spironolactone has been used as an ingredient in cosmetic skin and hair care compositions (U.S. Pat. App. Pub. No. 2010/0029574 and U.S. Pat. No. 7,879,910). Further, spironolactone has been used in the pharmaceutical industries to treat skin conditions (EP Pat. No. 0582458, PCT Pat. App. Pub. No. WO 2010/038234, and U.S. Pat. App. Pub. No. 2013/0143850), and for treating glaucoma (U.S. Pat. No. 3,551,554). The optical correction industry has also employed the use of aldosterone antagonists and methods for producing contact lenses have been described that employ spironolactone among other aldosterone antagonists (US Pat. App. Pub. No.: 2012/0113384). Other methods of using spironolactone are disclosed in U.S. Pat. No. 8,003,690, EP Pat. No. 0126684, U.S. Pat. App. Pub. Nos. 2010/0003354 and 2006/0210604, and PCT Pat. App. Pub. Nos. WO 2012/093117 and WO 2013/170317.

Spironolactone is used in the management of hyperaldosteronism, adolescent and adult acne, and female hirsutism. See also, Arita, R., Zavala, M., & Yee, R. W., “MGD Diagnosis,” Curr Opthalmol Rep, 49-57 (Jun. 4, 2014); Kim, G. K. and Del Rosso, J. Q., “Oral Spironolactone in Post-teenage Female Patients with Acne Vulgaris,” J Clin Aesthet Dermatol, 5(3): 37-50 (March 2012); Tavakkoli, F., “Review of the role of Spironolactone in the therapy of children,” 18th Expert Committee on the Selection and Use of Essential Medicines (Mar. 21, 2011).

To date, however, aldosterone antagonists, including spironolactone, have not been used for the treatment of ocular surface disorders relating to the eyelids and meibomian glands and in particular to treat Meibomian Gland Disease (MGD). Aldosterone antagonists are, as the name suggests, receptor antagonists at the mineralocorticoid receptor. Antagonism of these receptors inhibits sodium resorption in the collecting duct of the nephron in the kidneys. This interferes with sodium/potassium exchange, reducing urinary potassium excretion and weakly increasing water excretion (diuresis). Additionally, aldosterone antagonists, such as spironolactone, may also be employed for the purpose of reducing elevated or unwanted androgen activity in the body at its site of action and possibly demonstrating positive clinical effects on the glands of the eye and surrounding structures based on the clinical improvement noted from our patients using a topical eye application. Because of the multiple mechanisms of action of spironolactone, the inventors' data suggests another new mechanism of lipid production or secretion or affecting the lipid metabolism in a positive way to improve the oil content, inflammation, volume, and/or downregulating of the over inflammatory effects and thus improving the symptoms of MGD and the morbidity associated with MGD's chronic effects on the ocular surface and ocular surface's lid anatomy and pathophysiology. Based on the inventors' RT-PCR finding of upregulation of the ELOVL4 gene, another use of the non-toxic composition of spironolactone is in the posterior segment for the genetic disease Stargardt's disease and other retinal pathologies such as age-related macular degeneration.

As provided herein, aldosterone antagonists have been demonstrated to have clinical efficacy in patients who have a variety of symptoms and signs of MGD or meibomian gland related issues (e.g., based on Schirmer's testing, such as eyes that produce adequate amounts of tears (i.e. Schirmer's I test≥10) but have abnormal lipid or abnormal Meibomian Gland Disease. In particular, the inventors have found that aldosterone antagonists are useful for treating eye lid disease, including meibomian glands and disease associated therewith, as well as have a positive effect on the possible role of corneal epithelial cells in producing lipids. Examples of related efforts in this area include those described in U.S. Pat. Nos. 8,957,052; 3,711,602; 4,543,351; 8,709,393; 9,241,944; 9,610,294; 9,682,089; 9,730,948, U.S. Published Patent Application Nos. 2001/0019721; 2004/0043026; 2004/0067916; 2010/0068301; 2013/0131024; 2013/0143850; 2015/0216879; 2015/0342876; 2016/0082020; 2016/0136183; 2017/0273992; U.S. Reissue Pat. No. RE32112; European Patent Nos. EP0028525; EP0410348; EP0582458; and EP0603405; French Patent No. FR2588755; and International Patent Application Nos. WO00/72883; WO2006/002022; and WO2011/157798. Yet despite these efforts, there remains a need in the art for safe and effective therapies for treating ocular surface disorders relating to the eyelids and meibomian glands and in particular to treat Meibomian Gland Disease (MGD).

Spironolactone has been found to modulate the ELOVL4 gene and reasonably plays a role in retinal diseases such as Stargardt's macular dystrophy and age-related macular degeneration. Aldosterone antagonists, including spironolactone would be expected to increase production of very long-chain fatty acids (VLCFAs) produced by the ELOVL4 protein in corneal epithelial cells. These fatty acids can then be used to replace/supplement the oils not being produced by the Meibomian glands, which not only contributes to increased production of lipids but increases the quality of the lipids by substituting or replacing the lipids normally generated with different higher-quality lipids. ELOV4 may be involved as an important and possible causative or disease related gene for Meibomian Gland Disease. Severe forms of MGD may show abnormal gene regulation or missense mutations in this gene. Healthy cells such as meibocytes, ductal epithelial cells, corneal epithelial cells, conjunctival epithelial cells, progenitor cells, or pluripotent stem cells lacking the mutation can be cultured as donor cells for administration to a recipient (i.e. patient) with the mutation. Such ELOVL4 mutations may be corrected in the patient through gene therapy techniques. Appropriate genomic editing vectors can be designed to replace the mutated DNA directly in the patient with “healthy” DNA. Such vectors, as well as a donor template such as a plasmid or oligonucleotide, can be administered to the ocular surface or eyelids of a patient. The sgRNA and donor template are designed to insert the non-mutated ELOVL4 promoter in place of the mutated ELOVL4 promoter through homology-directed repair.

### SUMMARY OF THE INVENTION

The present disclosure extends the use of aldosterone antagonists to include use for treating ocular disease, such as ocular surface diseases, which includes the ocular region, including treatment of the eyelid for MGD and the posterior segment for conditions such as Stargardt's macular dystrophy and age-related macular degeneration.

Ocular disease that can be treated with methods and/or compositions of the invention can include any ophthalmic condition and or disease, including front of the eye diseases and/or back of the eye diseases, including any related or associated pathways involved in the disease process and treatment. The front of the eye diseases can deal with cellular or subcellular components of the front of the eye anatomy or histology, which includes the acellular tear film layer and its lipid aqueous mucin components. Front of the eye diseases also include diseases to the upper and lower eyelids including disease to the meibomian gland and its cellular and tissue components, (e.g., the muscle, lipid producing holocrine, exocrine and endocrine glands and its vascular and connective tissue components, etc.) as well as the conjunctiva and its associated cells including goblet cells, fibroblast cells, vascular and component blood cells. Front of the eye disorders further encompass any conditions or diseases of the corneal layers including the multi layers of epithelial cells, stromal cells and fibroblasts, corneal endothelial cells, corneal nerve and associated cells and ground substances. Diseases of the front of the eye could include, but is not limited to, inflammation, diffuse lamellar keratitis, corneal diseases, edemas, or opacifications with an exudative or inflammatory component, diseases of the eye that are related to systemic autoimmune diseases, any ocular surface disorders from dry eye (including ADDE, EDE, and chronic dry eye generally, keratoconjunctivitis, such as vernal keratoconjunctivitis, atopic keratoconjunctivitis and sicca keratoconjunctivitis), lid margin diseases, Meibomian Gland Disease or dysfunction, dysfunctional tear syndromes, anterior and/or posterior blepharitis, Staphylococcal blepharitis, microbial infection, computer vision syndrome (e.g., as well as any situations where users are staring at monitors, phones, e-readers, tablets, such as ipads, etc.), conjunctivitis (e.g., persistent allergic, giant papillary, seasonal intermittent allergic, perennial allergic, toxic, conjunctivitis caused by infection by bacteria, fungi, parasites, viruses or Chlamydia), conjunctival edema, anterior uveitis, and any inflammatory components or components of the aqueous fluid, inflammatory conditions resulting from surgeries such as LASIK®, LASEK®, refractive surgery, intraocular lens implantation (IOL), irreversible corneal edema as a complication of cataract surgery, edema as a result of insult or trauma (physical, chemical, pharmacological, etc.), genetic diseases of the cornea (corneal dystrophies including keratoconus, posterior polymorphous dystrophy; Fuch's dystrophies (corneal and endothelial), etc.), aphakic and pseudophakic bullous keratopathy, scleral diseases with or without inflammatory components, ocular cicatricial pemphigoid, pterygium, etc.

The back of the eye diseases can deal with cellular or subcellular components of the back of the eye anatomy and histology including the retina and all of the 10 or more cells comprising the layers of the retina, e.g., such as photoreceptors outer and inner layers, nuclear cell layers, amacrine and gangion cells, macula, fovea, and vitreous. Additional components of the back of the eye include the ciliary body, iris, uvea and the retinal pigment cells. Back of the eye diseases include processes that involve the optic nerve and its entire cellular and sub cellular components such as the axons and their innervations. These include diseases such as primary open angle glaucoma, acute and chronic closed angle glaucoma and any other secondary glaucomas. Diseases of the back of the eye also may include, but are not limited to, diseases of the optic nerve (including its cellular and sub cellular components such as the axons and their innervations), glaucomas (including primary open angle glaucoma, acute and chronic closed angle glaucoma and any other secondary glaucomas), myopic retinopathies, macular edema (including clinical macular edema or angiographic cystoid macular edema arising from various aetiologies such as diabetes, exudative macular degeneration and macular edema arising from laser treatment of the retina), diabetic retinopathy, age-related macular degeneration, retinopathy of prematurity, retinal ischemia and choroidal neovascularization and like diseases of the retina, genetic disease of the retina and macular degeneration, pars planitis, Posner Schlossman syndrome, Bechet's disease, Vogt-Koyanagi-Harada syndrome, hypersensitivity reactions, toxoplasmosis chorioretinitis, inflammatory pseudotumor of the orbit, chemosis, conjunctival venous congestion, periorbital cellulitis, acute dacryocystitis, non-specific vasculitis; sarcoidosis, cytomegalovirus infection, and the like. Genetic disease, e.g., Stargardt's retinal disease and age-related macular degeneration may also benefit from treatment with appropriate compositions of aldosterone antagonists, such as spironolactone. The inventors' non-toxic compositions may offer direct treatment to retinal pathology based on the inventors' new finding that spironolactone upregulates the RT-PCR for the enzyme of the ELOVL4 gene.

Compositions can comprise an effective amount of at least one aldosterone antagonist, as well as isomers, salts, and solvates thereof, and a carrier, such as a pharmaceutically acceptable carrier. The one or more aldosterone antagonists may be chosen from spironolactone, eplerenone, canrenone (e.g., canrenoate potassium), prorenone (e.g., prorenoate potassium), mexrenone (e.g., mexrenoate potassium), an acceptable isomer, salt or solvate thereof, or combinations comprising the same. Further, the pharmaceutically acceptable carrier may be any carrier. In particular aspects, the carrier may be or include any one or more of water, an aqueous solution, a polymer such as hydroxypropyl methylcellulose (hypromellose or HPMC), petrolatum, mineral oil, castor oil, carboxymethyl cellulose, polyvinyl alcohol, hydroxypropyl cellulose, hyaluronic acid (hyaluronan or HA), glycerin, polyvinyl alcohol, poly(acrylic acid), polycarbophil, polyethylene glycol (PEG) such as Polyethylene Glycol 200 (PEG 200), Polyethylene Glycol 300 (PEG 300), Polyethylene Glycol 400 (PEG 400), or any polyethylene glycol in liquid form, propylene glycol (PG), polysorbate 80, povidone, which may be otherwise referred to as povidone iodine, and/or dextran. The aldosterone antagonist can be present in the carrier by weight or by volume in an amount from 0.05% to 10%, or from 0.05% to 1%, or from 0.05% to 0.5%, or from 0.3% to 0.8% or from 0.4% to 1.2%, or from 0.6% to 1.5%, or from 1% to 2%, or from 3% to 4%, and so on. For example, the amount of aldosterone antagonist (such as at least one aldosterone antagonist, or isomer, salt, or solvate thereof selected from the group consisting of spironolactone, eplerenone, canrenone, prorenone, mexrenone, derivatives thereof, and combinations thereof) can range from above 0 mg/cc to 10 mg/cc, or from 0.0000025 mg/cc to 10 mg/cc, or from 0.000005 mg/cc to 10 mg/cc, 0.000005 mg/cc to 8 mg/cc, 0.000005 mg/cc to 6 mg/cc, 0.000005 mg/cc to 5 mg/cc, 0.000005 mg/cc to 4 mg/cc, 0.000005 mg/cc to 3 mg/cc, 0.000005 mg/cc to 2 mg/cc, 0.000005 mg/cc to 1 mg/cc, 0.000005 mg/cc to 0.5 mg/cc, 0.000005 mg/cc to 0.1 mg/cc, 0.000005 mg/cc to 0.05 mg/cc, 0.000005 mg/cc to 0.04 mg/cc, 0.000005 mg/cc to 0.03 mg/cc, 0.000005 mg/cc to 0.025 mg/cc, 0.000005 mg/cc to 0.02 mg/cc, 0.000005 mg/cc to 0.01 mg/cc, 0.000005 mg/cc to 0.009 mg/cc, 0.000005 mg/cc to 0.008 mg/cc, 0.000005 mg/cc to 0.007 mg/cc, 0.000005 mg/cc to 0.006 mg/cc, 0.000005 mg/cc up to 0.005 mg/cc, 0.000005 mg/cc to 0.004 mg/cc, 0.000005 mg/cc to 0.003 mg/cc, 0.000005 mg/cc to 0.0025 mg/cc, 0.000005 mg/cc to 0.002 mg/cc, 0.000005 mg/cc to 0.001 mg/cc, 0.000005 mg/cc to 0.0009 mg/cc, 0.000005 mg/cc to 0.0008 mg/cc, 0.000005 mg/cc to 0.0007 mg/cc, 0.000005 mg/cc to 0.0006 mg/cc, 0.000005 mg/cc to 0.0005 mg/cc, 0.000005 mg/cc to 0.0004 mg/cc, 0.000005 mg/cc to 0.0003 mg/cc, 0.000005 mg/cc to 0.0002 mg/cc, 0.000005 mg/cc to 0.00015 mg/cc, 0.000005 mg/cc to 0.0001 mg/cc, 0.000005 mg/cc to 0.00009 mg/cc, 0.000005 mg/cc to 0.00008 mg/cc, 0.000005 mg/cc to 0.00007 mg/cc, 0.000005 mg/cc to 0.00006 mg/cc, 0.000005 mg/cc to 0.00005 mg/cc, 0.000005 mg/cc to 0.00004 mg/cc, 0.000005 mg/cc to 0.00003 mg/cc, 0.000005 mg/cc to 0.000025 mg/cc, 0.000005 mg/cc to 0.00002 mg/cc, 0.000005 mg/cc to 0.000015 mg/cc, 0.000005 mg/cc to 0.00001 mg/cc, 0.000005 mg/cc to 0.000009 mg/cc, 0.000005 mg/cc to 0.000008 mg/cc, 0.000005 mg/cc to 0.000007 mg/cc, and 0.000005 mg/cc to 0.000006 mg/cc, or any ranges in between.

Still further, the amount of aldosterone antagonist (such as at least one aldosterone antagonist, or isomer, salt, or solvate thereof selected from the group consisting of spironolactone, eplerenone, canrenone, prorenone, mexrenone, derivatives thereof, and combinations thereof) can range from above 0 mg/cc and below 10 mg/cc, or below 9 mg/cc, or below 8 mg/cc, or below 7 mg/cc, or below 6 mg/cc, or below 5 mg/cc, or below 4 mg/cc, or below 3 mg/cc, or below 2 mg/cc, or below 1 mg/cc, or below 0.5 mg/cc, or below 0.1 mg/cc, or below 0.05 mg/cc, or below 0.04 mg/cc, or below 0.03 mg/cc, or below 0.025 mg/cc, or below 0.01 mg/cc, or from above 0 mg/cc to 0.000005 mg/cc, or from above 0 mg/cc to 0.009 mg/cc, to 0.008 mg/cc, to 0.007 mg/cc, to 0.006 mg/cc, up to 0.005 mg/cc, to 0.004 mg/cc, to 0.003 mg/cc, to 0.002 mg/cc, to 0.001 mg/cc, to 0.0009 mg/cc, to 0.0008 mg/cc, to 0.0007 mg/cc, to 0.0006 mg/cc, to 0.0005 mg/cc, to 0.0004 mg/cc, to 0.0003 mg/cc, to 0.0002 mg/cc, to 0.00015 mg/cc, to 0.0001 mg/cc, to 0.00009 mg/cc, to 0.00008 mg/cc, to 0.00007 mg/cc, to 0.00006 mg/cc, to 0.00005 mg/cc, to 0.00004 mg/cc, to 0.00003 mg/cc, to 0.000025 mg/cc, to 0.00002 mg/cc, to 0.000015 mg/cc, to 0.00001 mg/cc, to 0.000009 mg/cc, to 0.000008 mg/cc, to 0.000007 mg/cc, to 0.000006 mg/cc, or from above 0.0000025 mg/cc to below 0.005 mg/cc, or from above 0.0000025 mg/cc to below 0.0025 mg/cc.

Embodiments described herein may provide a composition consisting essentially of at least one aldosterone antagonist (including, isomers, salts, and solvates thereof), and a carrier, such as a pharmaceutically acceptable carrier, for example, PEG 300 and/or one or more pluronic.

The compositions described herein are useful in various physical forms. Non-limiting examples of acceptable compositional forms include liquids (e.g., eye drops), sprays, suspensions, gels, pastes, ointments, nanosized drug particles, pellets, emulsifications, creams, solids, etc. Pluronic can serve also as a carrier for the aldosterone antagonists such as spironolactone. This use of pluronic at concentrations within this range of 0.01% to 30% at cooler refrigerated temperature, e.g., 4 degrees with or without a therapeutic agent, e.g., spironolactone or other drug of interest, can be directly inserted or injected into a body lumen, e.g., the meibomian gland via the gland orifice such that the composition at the body temperature will create a stent to maintain the lumen from collapsing or closing, e.g., after meibomian gland duct dilation and or probing. Once the carrier is solidified, the pluronic will maintain the lumen during wound healing of the lumen as well as release the drug into the glandular region for an appropriate healing and therapeutic effect during the course of the ductal lumen healing after meibomian gland probing or dilation. One aspect of healing is to prevent the lumen from closing or scarring or development of an intraductal membrane. Other agents that can be administered in this manner include but are not limited to, e.g., testosterone, steroids, non steroidals, aldosterone antagonists, e.g., spironolactone, eplerenone, antibiotics, e.g., dapsone, tetracyclines, minocyclines, azithromycins or any antifibrotic agents, e.g., 5fu (fluorouracil), and/or mitomycin C.

It is another object of the embodiments described herein, to provide a method for treating an ocular surface disease, which includes the ocular or lid region, including treatment of the eyelid for MGD. The method comprises topically administering to an ocular region of an animal, such as a mammal (e.g., a human, canine, feline, etc.), a composition comprising an effective amount of at least one aldosterone antagonist (including isomers, salts, and solvates thereof) and a carrier, such as a pharmaceutically acceptable carrier to treat ocular surface disease, which includes the ocular or lid region, including treatment of the eyelid for MGD. Other routes of administration are also acceptable, such as intraductal injection. One option for intraductal injection is by injecting the composition with active agent with pluronic via a canula into the meibomian gland ductal system to maintain a stent-like action to maintain a patient lumen or enlarge an existing lumen during the injection. The compositions can comprise antibiotics and/or steroids or a steroid-like moiety. Compositions comprising an aldosterone antagonist, a steroid, such as prednisone, non-steroidal, antifibrotic, antifibrinolytic, and/or an antibiotic, such as dapsone, are included as embodiments of the invention. Method embodiments may include any of the compositions described herein.

### BRIEF DESCRIPTION OF THE DRAWINGS

The accompanying drawings illustrate certain aspects of embodiments of the present invention, and should not be used to limit the invention. Together with the written description the drawings serve to explain certain principles of the invention.

FIG. 1A is a bar graph showing baseline and follow-up Subjective Global Assessment for individual patients of the Pilot Study of Example 3. Zero to 10 on the y-axis. (Zero is no complaints and 10 is defined as the worse ocular surface complaint a patient has been experiencing). Red bar represents pre-treatment score. Purple bar represents post-treatment score.

FIG. 1B is a box plot of baseline and follow-up Subjective Global Assessment Scores of the Pilot Study of Example 3.

FIG. 2A is a bar graph showing baseline and follow-up Turbidity Scores for individual patients of the Pilot Study of Example 3. (Zero to 4 on the y-axis. Zero represents clear meibum and 4 represents toothpaste-like meibum). Red bar represents pre-treatment score. Purple bar represents post-treatment score.

FIG. 2B is a box plot of baseline and follow-up Turbidity Scores of the Pilot Study of Example 3.

FIG. 3A is a bar graph showing baseline and follow-up Zone A Scores for individual patients of the Pilot Study of Example 3. (Zero to 4 on the y-axis. Zero represents no vessels and 4 represents engorged vessels or vascular telangiectasias). Red bar represents pre-treatment score. Purple bar represents post-treatment score.

FIG. 3B is a box plot of baseline and follow-up Zone A Scores of the Pilot Study of Example 3.

FIGS. 4A-F are micrographs showing corneal epithelial cells of Example 6. FIGS. 4A-4C show microscopic images of control, 0.03 mg/ml, and 0.015 mg/ml treated cells, while FIGS. 4D-F show microscopic images of control, 0.03 mg/ml, and 0.015 mg/ml treated cells which were stained for lipids.

FIGS. 4G-H are micrographs showing corneal epithelial cells of Example 6. FIGS. 4G-4H show microscopic images of 0.006 mg/ml, and 0.003 mg/ml treated cells, while FIGS. 4I-4J show microscopic images of 0.006 mg/ml, and 0.003 mg/ml treated cells which were stained for lipids.

FIGS. 5A-L are micrographs showing corneal epithelial cells of Example 7. The top rows (FIGS. 5A-C, 5G-5I) represent unstained cells showing cell morphology, and the bottom rows (FIGS. 5D-F, 5J-5L) represent cells stained for lipids with Oil Red O. FIGS. 5A and 5D represent confluent morphology of the control group (no spironolactone). FIGS. 5B and 5E represent a 50× dilution showing non confluence and relative toxicity. FIGS. 5C and 5F represent a 100× dilution also showing non confluence and some relative toxicity. FIGS. 5G and 5J represent a 500× dilution, FIGS. 5H and 5K represent a 1000× dilution, and FIGS. 5I and 5L represent a 5000× dilution all demonstrating confluent nontoxic cell morphology.

FIG. 6 is a photographic image showing a normal (grade 0) Zone A in a patient. Zone A represents the region of the lid margin, 1 mm region posterior to the posterior edge of the lower lid suggesting normal anatomy and clinically suggesting little or no significant ocular surface irritation.

FIG. 7A is a photographic image showing non-symptomatic soft contact wearer in a 23 year old medical student. 7B is a photographic image showing a symptomatic soft contact wearer in a 23 year old medical student demonstrating significant vascularization suggesting a chronic ocular surface irritation of a given etiology.

FIGS. 8A-8D are photographic images showing different levels of Zone A, grades 1-4 of Zone A ocular irritation in a variety of patients.

FIG. 9 is a bar graph showing significant statistical measurements for baseline vs. first follow-up normal Schirmer's (n=75) for the clinical trial of Example 8.

FIG. 10 is a bar graph showing significant statistical measurements for baseline vs. first follow-up low Schirmer's (n=27) for the clinical trial of Example 8.

FIG. 11 is a bar graph showing significant statistical measurements for first follow-up vs. final follow-up normal Schirmer's (n=28) for the clinical trial of Example 8.

FIG. 12 is a bar graph showing significant statistical measurements for first follow-up vs. final follow-up high Schirmer's (n=10) for the clinical trial of Example 8.

FIG. 13 is a bar graph showing RT-PCR results of normalized ELOVL4 gene RT-PCR expression in the experiment of Example 9, in which corneal epithelial (HTCE) cells were treated with different dilutions of a 0.025 mg/mL spironolactone formulation.

FIGS. 14A and 14B are micrographs of a control eye (FIG. 14A) and eye with treatment of 0.025 mg/ml of preservative free spironolactone (FIG. 14B) administered twice a day for more than 25 days, showing no deleterious toxic effect in the normal in vivo animal model.

### BRIEF DESCRIPTION OF THE SEQUENCE LISTING

SEQ ID NO: 1 represents Homo sapiens ELOVL fatty acid elongase 4 (ELOVL4), RefSeqGene on chromosome 6 (public accession number NG_009108.1).

SEQ ID NO: 2 represents Homo sapiens ELOVL fatty acid elongase 4 (ELOVL4), mRNA (public accession number NM_022726.3).

SEQ ID NO: 3 represents Homo sapiens elongation of very long chain fatty acids protein 4 (public accession number NP_073563.1).

### DETAILED DESCRIPTION

Definitions:

As used herein, the singular forms “a”, “an” and “the” mean to include the plural forms as well, unless the context clearly indicates otherwise.

As used herein, the terms “administer(s)” “administered”, “administering” refers to the placement of a composition into a subject by a method or route which results in at least partial localization of the composition at a desired site such that desired effect is produced. A compound or composition described herein can be administered by any appropriate route known in the art including, but not limited to, topical administration (e.g., ophthalmic drops).

As used herein, the term “aldosterone antagonist(s)” means a compound that suppresses the receptor-mediated activity of aldosterone and/or mineralocorticoid receptors to predict factors which stimulate or suppress aldosterone secretion.

As used herein, the terms “carrier”, and “pharmaceutically acceptable carrier” may be used interchangeably, and mean any liquid, suspension, gel, salve, solvent, liquid, diluent, fluid ointment base, nanoparticle, liposome, micelle, giant micelle, and the like, which is suitable for use in contact with a subject without causing adverse physiological responses, and which does not interact with the other components of the composition in a deleterious manner. A number of carrier ingredients are known for use in making topical or injectable formulations, such as gelatin, polymers, fats and oils, lecithin, collagens, alcohols, water, etc. For example, injectables can be prepared, including compositions for injecting active agent into the meibomian gland orifice and/or the meibomian gland ducts. The term “pharmaceutically acceptable” means those compounds, materials, compositions, and dosage forms which are, within the scope of sound medical judgment, suitable for use in contact with the tissues of a subject without excessive toxicity, irritation, or other problem or complication, commensurate with a reasonable benefit/risk ratio.

As used herein, the term “isomer(s)” means all stereoisomers of the compounds and/or molecules referred to herein (e.g., aldosterone antagonists, such as spironolactone, eplerenone, canrenone, prorenone, mexrenone, etc., polymers, such as hydroxypropyl methylcellulose, etc.), including enantiomers, diastereomers, as well as all conformers, rotamers, and tautomers, unless otherwise indicated. The compounds and/or molecules disclosed herein include all enantiomers in either substantially pure levorotatory or dextrorotatory form, or in a racemic mixture, or in any ratio of enantiomers. Where embodiments disclose a (D)-enantiomer, that embodiment also includes the (L)-enantiomer; where embodiments disclose a (L)-enantiomer, that embodiment also includes the (D)-enantiomer. Where embodiments disclose a (+)-enantiomer, that embodiment also includes the (−)-enantiomer; where embodiments disclose a (−)-enantiomer, that embodiment also includes the (+)-enantiomer. Where embodiments disclose a (S)-enantiomer, that embodiment also includes the (R)-enantiomer; where embodiments disclose a (R)-enantiomer, that embodiment also includes the (S)-enantiomer. Embodiments are intended to include any diastereomers of the compounds and/or molecules referred to herein in diastereomerically pure form and in the form of mixtures in all ratios. Unless stereochemistry is explicitly indicated in a chemical structure or chemical name, the chemical structure or chemical name is intended to embrace all possible stereoisomers, conformers, rotamers, and tautomers of compounds and/or molecules depicted.

As used herein, the terms “treat”, “treating”, or “treatment(s)” means the application or administration of a composition described herein, or identified by a method described herein, to a subject, or application or administration of the therapeutic agent to an isolated tissue or cell line from a subject, who has a disease, a symptom of disease, or a predisposition toward a disease, with the purpose to cure, heal, alleviate, relieve, alter, remedy, ameliorate, improve, or affect the disease, the symptoms of disease, or the predisposition toward disease. As used herein, the term “subject” refers to an animal, such as a mammal, including for example a human or domesticated animal (e.g., a dog or cat), which is to be the recipient of a particular treatment.

As used herein, terms “effective amount”, “therapeutically effective amount”, or “pharmaceutically effective amount” may be used interchangeably and mean the amount of a compound that, when administered to a subject for treating a state, disorder or condition, is sufficient to effect such treatment. The “effective amount” will vary depending on the compound, the disease and its severity, and the age, weight, physical condition and responsiveness of the mammal to be treated.

As used herein, the terms “ocular” or “ocular region” means the eye, surrounding tissues of the eye, and to bodily fluids in the region of the eye, including the periocular region. Specifically, the term includes the cornea or, the sclera or, the uvea, the conjunctiva (e.g., bulbar conjunctiva, palpebral conjunctiva, and tarsal conjunctiva), anterior chamber, lacrimal sac, lacrimal canals, lacrimal ducts, medial canthus, nasolacrimal duct, and the eyelids (e.g., upper eyelid and lower eyelid). Additionally, the term includes the inner surface of the eye (conjunctiva overlying the sclera), and the inner surface of the eyelids (e.g., the palpebral conjunctiva).

As used herein, the term “conjunctiva” means the mucous membrane lining the inner surfaces of the eyelids and anterior part of the sclera.

As used herein, the term “cornea” means the clear central frontal tissue of the eye, including its six layers. One such layer is the corneal epithelium, which is comprised of a stratified squamous non-keratinized epithelium.

As used herein, the term “eye(s)” means the light sensing organs of a subject and can refer to the sense organ providing vision to a subject.

As used herein, the term “eyelid” means a movable cover over the eye, which may further comprise eyelashes and ciliary and meibomian glands along its margin. The eyelid consists of loose connective tissue containing a thin plate of fibrous tissue lined with mucous membrane (conjunctiva).

As used herein, “meibomian gland” refers to one of several sebaceous glands that secrete sebum from their ducts on the posterior margin of each eyelid. The glands are embedded adjacent to the tarsal plate of each eyelid and include ductal and periductal components.

As used herein, the term “canthus” means either corner of the eye where the upper and lower eyelids meet.

As used herein, the term “mucus” means the viscous, slippery secretions of mucous membranes and glands, containing mucin, white blood cells, water, inorganic salts, and exfoliated cells.

As used herein, the term “lacrimal apparatus” refers to one or more of a lacrimal gland, lacrimal duct, lacrimal sac, or lacrimal canal, or any organ associated with the production or drainage of tears.

As used herein, the term “sclera” means the collagenous outer-wall of the eyeball comprising mostly collagen and some elastic tissue, which is covered by conjunctiva. In humans, the sclera is sometimes referred to as the white of the eye.

As used herein, the term “tear(s)” means the liquid produced by lacrimation, for cleaning and lubricating the eyes. Tear film is composed of a lipid/oil layer (secreted from meibomian glands), an aqueous layer and a mucous layer. The function of the lipid/oil layer is to slow the evaporation of the tear fluid. “Schirmer's I test” is a clinical procedure for measuring adequate tear production using a strip of filter paper without anesthesia; a negative test result when the strip measurement is less than or equal to 10 mm of moisture on the filter paper in 5 minutes).

Compositions

The compositions disclosed comprise an effective amount, such as a pharmaceutically effective amount, of at least one aldosterone antagonist, including isomers, salts, and solvates thereof, as described herein and a carrier, such as a pharmaceutically effective carrier, for administration to an ocular or lid region of a subject to treat an ocular surface disease, which can include treatment of the eyelid for MGD.

In a particular aspect, the compositions disclosed consist essentially of an effective amount, such as a pharmaceutically effective amount, of at least one aldosterone antagonist, including isomers, salts, and solvates thereof, as described herein and a carrier such as a pharmaceutically effective carrier for administration to an ocular region and/or lid region, such as the eyelids, of a subject to treat an ocular surface disease, such as MGD. These formulations are more likely also to be compatible to the very sensitive cell of the retina and can be administered directly by injection into the eye or by suprachoroidal routes.

The compositions may be in the form of a liquid (e.g., an ophthalmic drop or an intraductal or ductal orifice injectable), a suspension, a gel, a slurry, an ointment, a cream, an emulsion, a solid, a powder of variable sizes macro to nano particle sized (wettable powder or dry powder), or a pellet. In particular aspects, the composition is a liquid composition. Another aspect is the composition is using pluronic. At given concentrations 0.01% to 30% at colder temperatures, pluronic with or without a therapeutic agent when reaching the body or room temperature will solidify and act as a stent or a dilator for the intraductal or ductal orifice to enhance the maintenance of the lumen, reestablish it and be a source of drug delivery to the meibomian glands.

In particular embodiments, the at least one aldosterone antagonists may be chosen from spironolactone, eplerenone, canrenone (e.g., canrenoate potassium), prorenone (e.g., prorenoate potassium), mexrenone (e.g., mexrenoate potassium), an acceptable isomer, salt or solvate thereof, or combinations comprising the same. Further, the pharmaceutically acceptable carrier may be any carrier. In particular aspects, the carrier may be any one or more of water, an aqueous solution, a polymer such as hydroxypropyl methylcellulose (hypromellose or HPMC), pluronic, petrolatum, mineral oil, carboxymethyl cellulose, polyvinyl alcohol, hydroxypropyl cellulose, hyaluronic acid (hyaluronan or HA), glycerin, polyvinyl alcohol, polyethylene glycol (PEG) such as PEG 300, PEG 200, Polyethylene Glycol 400 (PEG 400), propylene glycol (PG), polysorbate 80, povidone, which may be otherwise referred to as povidone iodine, and/or dextran. The aldosterone antagonist can be present in the carrier by weight or by volume in an amount from 0.025 mg/cc or lower, such as 0.0005 mg/cc, or 0.00005 mg/cc, or 0.000005 mg/cc, or from 0.05% to 10%, such as from 0.05% to 1%, or from 0.05% to 0.5%, or from 0.3% to 0.8% or from 0.4% to 1.2%, or from 0.6% to 1.5%, or from 1% to 2%, or from 3% to 4%, and so on. Further, for example, the amount of aldosterone antagonist (such as at least one aldosterone antagonist, or isomer, salt, or solvate thereof selected from the group consisting of spironolactone, eplerenone, canrenone, prorenone, mexrenone, derivatives thereof, and combinations thereof) can range from above 0 mg/cc to 10 mg/cc, or from 0.0000025 mg/cc to 10 mg/cc, or from 0.000005 mg/cc to 10 mg/cc, 0.000005 mg/cc to 8 mg/cc, 0.000005 mg/cc to 6 mg/cc, 0.000005 mg/cc to 5 mg/cc, 0.000005 mg/cc to 4 mg/cc, 0.000005 mg/cc to 3 mg/cc, 0.000005 mg/cc to 2 mg/cc, 0.000005 mg/cc to 1 mg/cc, 0.000005 mg/cc to 0.5 mg/cc, 0.000005 mg/cc to 0.1 mg/cc, 0.000005 mg/cc to 0.05 mg/cc, 0.000005 mg/cc to 0.04 mg/cc, 0.000005 mg/cc to 0.03 mg/cc, 0.000005 mg/cc to 0.025 mg/cc, 0.000005 mg/cc to 0.02 mg/cc, 0.000005 mg/cc to 0.01 mg/cc, 0.000005 mg/cc to 0.009 mg/cc, 0.000005 mg/cc to 0.008 mg/cc, 0.000005 mg/cc to 0.007 mg/cc, 0.000005 mg/cc to 0.006 mg/cc, 0.000005 mg/cc up to 0.005 mg/cc, 0.000005 mg/cc to 0.004 mg/cc, 0.000005 mg/cc to 0.003 mg/cc, 0.000005 mg/cc to 0.0025 mg/cc, 0.000005 mg/cc to 0.002 mg/cc, 0.000005 mg/cc to 0.001 mg/cc, 0.000005 mg/cc to 0.0009 mg/cc, 0.000005 mg/cc to 0.0008 mg/cc, 0.000005 mg/cc to 0.0007 mg/cc, 0.000005 mg/cc to 0.0006 mg/cc, 0.000005 mg/cc to 0.0005 mg/cc, 0.000005 mg/cc to 0.0004 mg/cc, 0.000005 mg/cc to 0.0003 mg/cc, 0.000005 mg/cc to 0.0002 mg/cc, 0.000005 mg/cc to 0.00015 mg/cc, 0.000005 mg/cc to 0.0001 mg/cc, 0.000005 mg/cc to 0.00009 mg/cc, 0.000005 mg/cc to 0.00008 mg/cc, 0.000005 mg/cc to 0.00007 mg/cc, 0.000005 mg/cc to 0.00006 mg/cc, 0.000005 mg/cc to 0.00005 mg/cc, 0.000005 mg/cc to 0.00004 mg/cc, 0.000005 mg/cc to 0.00003 mg/cc, 0.000005 mg/cc to 0.000025 mg/cc, 0.000005 mg/cc to 0.00002 mg/cc, 0.000005 mg/cc to 0.000015 mg/cc, 0.000005 mg/cc to 0.00001 mg/cc, 0.000005 mg/cc to 0.000009 mg/cc, 0.000005 mg/cc to 0.000008 mg/cc, 0.000005 mg/cc to 0.000007 mg/cc, and 0.000005 mg/cc to 0.000006 mg/cc, or any ranges in between.

Still further, the amount of aldosterone antagonist (such as at least one aldosterone antagonist, or isomer, salt, or solvate thereof selected from the group consisting of spironolactone, eplerenone, canrenone, prorenone, mexrenone, derivatives thereof, and combinations thereof) can range from above 0 mg/cc and below 10 mg/cc, or below 9 mg/cc, or below 8 mg/cc, or below 7 mg/cc, or below 6 mg/cc, or below 5 mg/cc, or below 4 mg/cc, or below 3 mg/cc, or below 2 mg/cc, or below 1 mg/cc, or below 0.5 mg/cc, or below 0.1 mg/cc, or below 0.05 mg/cc, or below 0.04 mg/cc, or below 0.03 mg/cc, or below 0.025 mg/cc, or below 0.01 mg/cc, from above 0 mg/cc to 0.000005 mg/cc, or from above 0 mg/cc to 0.009 mg/cc, to 0.008 mg/cc, to 0.007 mg/cc, to 0.006 mg/cc, up to 0.005 mg/cc, to 0.004 mg/cc, to 0.003 mg/cc, to 0.002 mg/cc, to 0.001 mg/cc, to 0.0009 mg/cc, to 0.0008 mg/cc, to 0.0007 mg/cc, to 0.0006 mg/cc, to 0.0005 mg/cc, to 0.0004 mg/cc, to 0.0003 mg/cc, to 0.0002 mg/cc, to 0.00015 mg/cc, to 0.0001 mg/cc, to 0.00009 mg/cc, to 0.00008 mg/cc, to 0.00007 mg/cc, to 0.00006 mg/cc, to 0.00005 mg/cc, to 0.00004 mg/cc, to 0.00003 mg/cc, to 0.000025 mg/cc, to 0.00002 mg/cc, to 0.000015 mg/cc, to 0.00001 mg/cc, to 0.000009 mg/cc, to 0.000008 mg/cc, to 0.000007 mg/cc, to 0.000006 mg/cc, or from above 0.00001 mg/cc to below 0.005 mg/cc, or from above 0.00001 mg/cc to below 0.0025 mg/cc.

Aldosterone Antagonists:

As disclosed throughout, compositions described herein comprise at least one aldosterone antagonist. The aldosterone antagonists may be a natural aldosterone antagonist, (i.e., not synthetically produced), a synthetic aldosterone antagonist (e.g., a chemically synthesized aldosterone antagonist) or combinations thereof. In embodiments, the aldosterone antagonists can include one or more of the aldosterone antagonists disclosed for example in U.S. Pat. Nos. 9,241,944 and/or 9,682,089, which are incorporated by reference herein in their entireties.

In embodiments, aldosterone antagonist(s) may be any aldosterone antagonist or derivative thereof known in the art, including non-limiting representative examples provided in U.S. Pat. No. 4,192,802, U.S. Pat. App. Pub. No. 2003/0199483, U.S. Pat. App. Pub. Nos. 2004/0102423 and 2009/0325918, EP 0046291, and WO 2004/085458, all of which are incorporated by reference herein in their entireties.

Aldosterone antagonists and derivatives can have the following structure:

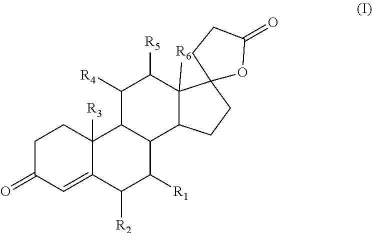

wherein R₁, R₂, R₃, R₄, R₅, and R₆ may each independently represent a hydrogen atom, an oxygen atom, a halogen atom (e.g., fluorine, chlorine, bromine, iodine), a saturated or unsaturated, branched or unbranched, substituted or unsubstituted aliphatic or aromatic hydrocarbon containing between 1 and 20 carbon atoms, such as an alkyl group, an alkenyl group, an alkynyl group, an alkoxy group, an acyl group, an acetyl group, an aryl group, an aryloxy group, an acrylyl group, a carbonyl group, a cycloalkyl group, a hydroxyalkyl group, an alkoxyalkyl group, a hydroxycarbonyl group, an alkoxycarbonyl group, an acyloxyalkyl group, a heteroaryl group, a heterocyclyl group, a ketal group, an acetal group, an amine group, an amide group, an imide group, an azide group, a sulfur-containing group, a thiol group, a sulfide group, a disulfide group, a sulfinyl group, a sulfonyl group, an acetylthio group, a formyl group, a furyl group, a hydroxyl group, a hetero atom, a cyano group, or an ester, ether, ketone, or aldehyde functional group, as well as substituted groups thereof. When R₁ and R₂ are each a hydrogen atom, there is a C—C double bond present between the carbon atoms to which R₁ and R₂ are attached.

For example, in particular embodiments R₁, R₂, R₃, R₄, R₅, and R₆ may each independently represent a methyl group, an ethyl group, a propyl group, a butyl group, a cyclopropyl group, a cyclobutyl group, a cyclopentyl group, a cyclohexyl group, a cycloheptyl group, a methoxy group, an ethoxy group, a propoxy group, a butoxy group, an acetyl group, a propionyl group, a butyryl group, a hydroxymethyl group, a hydroxyethyl group, a hydroxypropyl group, a hydroxybutyl group, a methoxymethyl group, a methoxyethyl group, a methoxypropyl group, a methoxybutyl group, an ethoxy group, an ethoxymethyl group, an ethoxyethyl group, an ethoxypropyl group, an ethoxybutyl group, a propoxymethyl group, a propoxyethyl group, a propoxypropyl group, a propoxybutyl group, a butoxymethyl group, a butoxyethyl group, a butoxypropyl group, a butoxybutyl group, a methoxycarbonyl group, an ethoxycarbonyl group, a propoxycarbonyl group, a butoxycarbonyl group, an acetoxymethyl group, an acetoxyethyl group, an acetoxypropyl group, an acetoxybutyl group, a propionyloxymethyl group, a propionyloxyethyl group, a butyryloxymethyl group, a butyryloxyethyl group, a phenoxy group, a phenyl group, a benzyl group, a benzoyl group, a benzoxy group, a pyrrolyl group, an imidazolyl group, a thiazolyl group, a pyridyl group, a pyrimidyl group, an oxazolyl group, an acetylthio group, a furyl group, a thienyl group, an epoxy group, or substituted groups thereof.

In one embodiment, the at least one aldosterone antagonist is one or more aldosterone antagonist selected from the group consisting of spironolactone, eplerenone, canrenone, prorenone, mexrenone, and combinations thereof, as well as isomers, salts, and solvates thereof

In a particular aspect, the at least one aldosterone antagonist is spironolactone, including derivatives, isomers, salts, and solvates thereof. Spironolactone is an aldosterone antagonist of Structure (I) above where R₁ is an acetylthio group, R₃ and R₆ are each a methyl group, and R₂, R₄, and R₅ are each a hydrogen atom. Spironolactone derivatives comprising Structure II (below) and any one or more of the substituents mentioned above for Structure I are also included. Spironolactone refers to aldactone, 3-(3-oxo-7a-acetylthio-17/3-hydroxy-androst-4-en-17a-yl) propiolactone. Spironolactone, commercially available as ALDACTONE® from Pfizer and also referred to as 7α-acetylthio-3-oxo-17α-pregn-4-ene-21,17-carbolactone or 17-hydroxy-7α-mercapto-3-oxo-17α-pregn-4-ene-21-carboxylic acid, γ-lactone acetate, has the molecular formula C₂₄H₃₂O₄S and a molar mass of 416.574 g mol⁻¹. Spironolactone has the following structure (II):

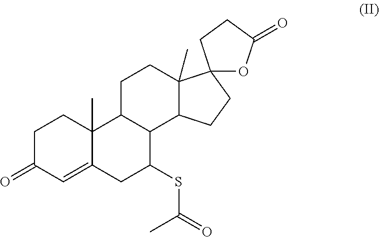 [SMILES: CC(=O)SC1CC2=CC(=O)CCC2(C)C2CCC3(C)C(CCC34CCC(=O)O4)C12]

In particular embodiments, R₁ of Structure I above can be an ester functional group namely —COOR′, R₂ is a hydrogen atom, R₃ is a methyl group, R₄ is an oxygen atom and forms a 3-membered heterocyclic ring together with the carbon atom to which it is attached and an adjacent carbon atom, R₅ is a hydrogen atom, and R₆ is a methyl group. In a particular aspect, R′ of the —COOR' group can be a C₁₋₁₀ alkyl group, such as a methyl ethyl, propyl, or butyl group.

In another particular aspect, the at least one aldosterone antagonist is eplerenone, including derivatives, isomers, salts, and solvates thereof. Eplerenone is an aldosterone antagonist of Structure (I) above where R₁ is a —COOR′ group, R₂ and R₅ are each a hydrogen atom, R′, R₃, and R₆ are each a methyl group, and R₄ is an oxygen atom that forms a 3-membered heterocyclic ring together with the carbon atom of the ring to which it is attached and an adjacent carbon atom in the ring. Eplerenone derivatives comprising Structure III (below) and any one or more of the substituents mentioned above for Structure I are also included. Eplerenone is commercially available as INSPRA® from Pfizer, also referred to as pregn-4-ene-7,21-dicarboxylic acid, 9,11-epoxy-17-hydroxy-3-oxo, γ-lactone, methyl ester (7α, 11α, 17α), has the molecular formula C₂₄H₃₀O₆, a molar mass of 414.49 g mol⁻¹, and the following structure (III):

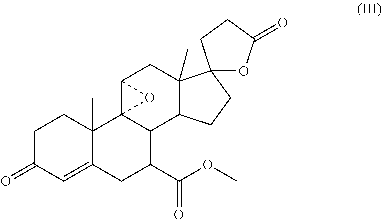 [SMILES: COC(=O)C1CC2=CC(=O)CCC2(C)[C@@]23O[C@@H]2CC2(C)C(CCC24CCC(=O)O4)C13]

In another particular aspect, the at least one aldosterone antagonist is canrenone, including derivatives, isomers, salts, and solvates thereof. Canrenone is an aldosterone antagonist of Structure (I) above where R₃ and R₆ are each a methyl group, R₁ and R₂ are each a hydrogen atom, and there is a double bond present between the carbon atom on which is attached Ri and the carbon atom on which is attached R₂. Canrenone derivatives comprising Structure IV (below) and any one or more of the substituents mentioned above for Structure I are also included. Canrenone may otherwise be referred to as 10,13-dimethylspiro[2,8,9,11,12,14,15,16-octahydro-1H-cyclopenta[a]phenanthrene-17,5′-oxolane]-2′,3-dione, has the molecular formula C₂₂H₂₈O₃, a molar mass of about 340.456 g mol⁻¹, and has the following structure (IV):

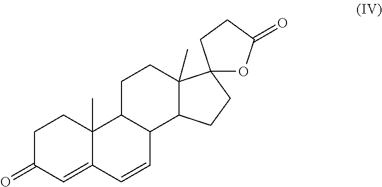 [SMILES: CC12CCC(=O)C=C1C=CC1C2CCC2(C)C1CCC21CCC(=O)O1]

In another particular aspect, the at least one aldosterone antagonist is prorenone, including derivatives, isomers, salts, and solvates thereof. Prorenone is an aldosterone antagonist of Structure (I) above with no C—C double bonds and where R₁ is a C₁ alkyl group (CH₂) and forms a 3-membered ring together with the carbon atom of the ring to which it is attached and an adjacent carbon atom in the ring, R₃ and R₆ are each a methyl group, R₂, R₄ and R₅ are each a hydrogen atom. Prorenone derivatives comprising Structure V (below) and any one or more of the substituents mentioned above for Structure I are also included. Prorenone, also referred to as 3-(17β-hydroxy-6β,7β-methylene-3-oxo-4-androsten-17α-yl)propionic acid γ-lactone, has the molecular formula C₂₃H₃₀O₃, a molar mass of about 354.48 g mol⁻¹, and the following structure (V):

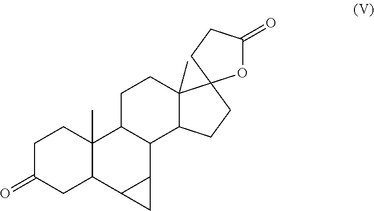 [SMILES: CC12CCC(=O)CC1C1CC1C1C2CCC2(C)C1CCC21CCC(=O)O1]

In another particular aspect, the at least one aldosterone antagonist is mexrenone, including derivatives, isomers, salts, and solvates thereof. Mexrenone is an aldosterone antagonist of Structure (I) above where R₁ is a —COOR′ group, R₂, R₄, and R₅ are each a hydrogen atom, R′, R₃, and R₆ are each a methyl group. Mexrenone derivatives comprising Structure VI (below) and any one or more of the substituents mentioned above for Structure I are also included. Mexrenone, also referred to as 17-hydroxy-3-oxo-17α-pregn-4-ene-7α,21-dicarboxylic acid 7-methyl ester gamma-lactone, has the molecular formula C₂₄H₃₂O₅ and a molar mass of about 400.51 g mol⁻¹. Mexrenone has the following structure (VI):

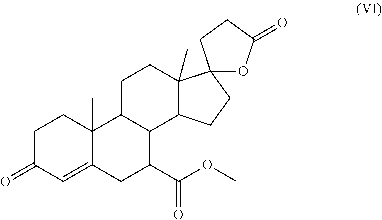 [SMILES: COC(=O)C1CC2=CC(=O)CCC2(C)C2CCC3(C)C(CCC34CCC(=O)O4)C12]

In a particular aspect, the at least one aldosterone antagonists used in the compositions described herein may be at least two of the above aldosterone antagonists (i.e., at least two of spironolactone, eplerenone, canrenone, prorenone, mexrenone), at least three of the above aldosterone antagonists, at least four of the above aldosterone antagonists, up to and including all of the above aldosterone antagonists, including isomers, salts, and solvates thereof.

Pharmaceutically Acceptable Carriers:

The carriers (e.g., pharmaceutically acceptable carriers) described herein will allow the one or more aldosterone antagonist(s) to remain efficacious (e.g., capable of treating an ocular surface disease which includes the ocular or lid region, including treatment of the eyelid for MGD). Non-limiting examples of carriers described herein include liquids, suspensions, gels, ointments, nanosized drug particles, pellets, slurries, or solids (including wettable powders or dry powders). The selection of the carrier material will depend on the intended application. One goal is to provide formulations with little to no burning or stinging, or reduced burning or stinging.

Carriers and pharmaceutically acceptable carriers for use with the compositions of the present invention are well known in the pharmaceutical arts. Non-limiting examples of such carriers include such vehicles as water; organic solvents, alcohols, lower alcohols that are readily capable of evaporating from the skin, ethanol, glycols, glycerin, aliphatic alcohols, mixtures of water and organic solvents, mixtures of water and alcohol, mixtures of organic solvents such as alcohol and glycerin, lipid-based materials such as fatty acids, acylglycerols, oils, mineral oils, fats of natural or synthetic origin, phosphoglycerides, sphingolipids, waxes, DMSO, protein-based materials such as collagen and gelatin, volatile and/or non-volatile silicon-based materials, cyclomethicone, dimethiconol, dimethicone copolyol (Dow Corning, Midland, Mich., USA), hydrocarbon-based materials such as petrolatum and squalane, sustained-release vehicles such as microsponges and polymer matrices, suspending agents, emulsifying agents, and other vehicles and vehicle components that are suitable for administration to the ocular region, as well as mixtures of topical vehicle components as identified above or otherwise known to the art.

Carriers such as those known in the art may be useful in delivering the active ingredient of the invention to the area of interest. Such carriers include liposomes, polymeric micelles, microspheres, and nanoparticles. The active ingredient of the invention can be dispersed or emulsified in a medium in a conventional manner to form a liquid preparation or mixed with a semi-solid (gel) or solid carrier to form a paste, powder, ointment, cream, lotion or the like. Other examples of suitable pharmaceutically acceptable carriers include water, buffered saline, petroleum jelly (vaseline), petrolatum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, synthetic polymers, alcohols, polyols, salt solutions, alcohol, silicone, waxes, polyethylene glycols, propylene glycol, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyinylpyrrolidone, and the like. Examples of suitable carriers for sustained or delayed release in a moist environment include gelatin, gum arabic, xanthane polymers. Pharmaceutical carriers capable of releasing the active ingredient of the invention when exposed to any oily, fatty, waxy, or moist environment on the area being treated, include thermoplastic or flexible thermoset resin or elastomer including thermoplastic resins such as polyvinyl halides, polyvinyl esters, polyvinylidene halides and halogenated polyolefins, elastomers such as brasiliensis, polydienes, and halogenated natural and synthetic rubbers, and flexible thermoset resins such as polyurethanes, epoxy resins and the like. Controlled delivery systems are described, for example, in U.S. Pat. No. 5,427,778 which provides gel formulations and viscous solutions for delivery of the active ingredient of the invention to a wound site. Gels have the advantages of having a high water content to keep the wound moist, the ability to absorb wound exudate, easy application and easy removal by washing. Preferably, the sustained or delayed release carrier is a gel, liposome, microsponge or microsphere.

In a particular embodiment, the pharmaceutical composition is an ophthalmic drop or an ophthalmic ointment. Common ingredients of such ophthalmic drops or ointments have been reviewed (see The Pharmaceutical Journal, PJ June 2017 online, online |URI: 20202915; M. B. Abelson et al., “The Foundation of a Good Formulation”, Review of Ophthalmology, published online 3 Mar. 2017; and Rajasekaran A, et al. “A comparative review on conventional and advanced ocular drug delivery formulations”. International Journal of PharmTech Research 2010; 2:1:668-674; each incorporated herein by reference). The eye drop is provided in any formulation generally used, for example, in the form of an aqueous eye drop such as aqueous eye drop solution, aqueous eye drop suspension, viscous eye drop solution, solubilized eye drop solution and the like, or in the form of a non-aqueous eye drop such as a non-aqueous eye drop solution, non-aqueous eye drop suspension and the like. When the composition for treating cornea of the present invention is prepared as an aqueous eye drop, it preferably contains an additive which is usually used in an aqueous eye drop. The examples of such an additive include preservatives, isotonic agents, buffering agents, stabilizer, pH regulators or the like. When the composition is used in a form of an eye ointment, it includes any formulations usually used. For example, it can be easily produced by optionally heating an eye ointment base and mixing it with an active ingredient of the invention. The active ingredient of the invention may be optionally dissolved or suspended in a suitable solvent, for example, sterilized pure water, distilled water for injection, vegetable oil such as castor oil and the like, before mixing with the eye ointment base. The examples of the eye ointment base agent include purified lanolin, Vaseline, plastibase, liquid paraffin and the like. The above-mentioned preservative, stabilizer and the like can be optionally blended provided the object of the present invention is not hurt.

For ophthalmic delivery, an active ingredient may be combined with ophthalmologically acceptable preservatives, co-solvents, surfactants, viscosity enhancers, penetration enhancers, buffers, sodium chloride, or water to form an aqueous, sterile ophthalmic suspension or solution. Solution formulations may be prepared by dissolving the active ingredient in a physiologically acceptable isotonic aqueous buffer. Further, the solution may include an acceptable surfactant to assist in dissolving the active ingredient.

Viscosity enhancers, such as hydroxy methyl cellulose, hydroxy ethyl cellulose, sodium carboxy methyl cellulose, hydroxypropyl methyl cellulose, polyalcohol, and polyvinylpyrrolidone, or the like may be added to the compositions of the present invention to improve the retention of the compound. Other examples of viscosity enhancers include polyvinyl alcohol, poloxamers, hyaluronic acid, carbomers, and polysaccharides, that is, cellulose derivatives, gellan gum, and xanthan gum.

Examples of permeation or penetration enhancers include benzalkonium chloride, polyoxyethylene glycol ethers (lauryl, stearyl and oleyl), ethylenediaminetetra acetic acid sodium salt, sodium taurocholate, saponins, bile salts, and cremophor EL. Other examples of permeation enhancers include, for example, dimethylsulfoxide (DMSO), dimethyl formamide (DMF), N,N-dimethylacetamide (DMA), decylmethylsulfoxide (C10MSO), polyethylene glycol monolaurate (PEGML), glyceral monolaurate, lecithin, 1-substituted azacycloheptan-2-ones, particularly 1-N-dodecylcyclazacylcoheptan-2-ones (available under the trademark Azone™ from Nelson Research & Development Co., Irvine, Calif.), alcohols and the like.

In some embodiments, Durezol (difluprednate) is added as an emulsifying agent. Other additives that improve solubility that may be added include certain surfactants, caffeine, and nicotinamide derivatives. In some embodiments, cyclodextrins may be included in the formulations to act as carriers for hydrophobic drug molecules in aqueous solution.

In order to prepare a sterile ophthalmic ointment formulation, the active ingredient is combined with a preservative in an appropriate vehicle, such as mineral oil, liquid lanolin, or white petrolatum. Sterile ophthalmic gel formulations may be prepared by suspending the active ingredient in a hydrophilic base prepared from the combination of, for example, CARBOPOL®-940 (BF Goodrich, Charlotte, N.C.), or the like, according to methods known in the art. VISCOAT® (Alcon Laboratories, Inc., Fort Worth, Tex.) may be used for intraocular injection, for example. Other compositions of the present invention may contain penetration enhancing agents such as cremophor and TWEEN® 80 (polyoxyethylene sorbitan monolaureate, Sigma Aldrich, St. Louis, Mo.), in the event the active ingredient is less penetrating in the eye.

Additional embodiments include enhancers such as oleic acid, 1-methyl-2 pyrrolidone, 2,2-dimethyl octanoic acid and N,N dimethyl lauramide/propylene glycol monolaureate or combinations thereof as described in European Patent No. EP0582458B1.

Other examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (1) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As solvents volatile monohydric aliphatic alcohols can be used, such as ethanol and propanol. Further favorable solvents are di- and polyhydric alcohols, such as glycerol, propylene glycol, and polyethylene glycol, because, besides functioning as solvents, they at the same time function as humectants of the skin, which is adapted to improve the absorption. A particularly favorable solvent is propylene glycol, which additionally also functions as a bactericidal agent.

Other vehicles can include crotamiton, glycol monosalicylate, peppermint oil Methyl salicylate, sesame oil, beeswax, liquid paraffin, squalene, vaseline, ethanol, isopropanol, methanol, 2-octyldodecanol, 1,3-butylene glycol, polyethylene glycol 200, 300 or 400, isopropyl myristate, diisopropyl adipate, octadodecyl myristate, isopropyl palmitate, butyl stearate, diethyl sebacate, glyceryl tricaprate, propylene glycol didecanate, and purified water.

Other ingredients in the formulation may include cetearyl alcohol, sodium lauryl sulfate, glycerol monostearate, polyoxyethylene stearate mixture, light mineral oil, diisopropyl adipate, white petrolatum, propyl-p-hydroxybenzoate (propyl paraben), methyl-p-hydroxybenzoate (methyl paraben), sodium citrate dihydrate, citric acid monohydrate, and purified water, USP. Dyes and colorants may also be used, such as trypan blue or methylene blue.

Any of the well-known techniques, carriers, and excipients are suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co. Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

The carrier may also be a commercially available neutral base known in the art. A neutral base has no significant therapeutic effect of its own. It simply conveys the active pharmaceutical ingredient, although some vehicles may do so with greater ease or effectiveness than others. A neutral base may be a cream used cosmetically for softening and/or cleaning the skin. Non-limiting examples include EUCERIN® (Beiersdorf Aktiengesellschaft Corp., Hamburg, Germany), AQUAPHOR® (Beiersdorf Aktiengesellschaft Corp., Hamburg, Germany), and liposomal vehicles. A preferred neutral base is VANICREAM® (Pharmaceutical Specialties, Inc., Rochester, Minn., USA). VANICREAM® is composed of purified water, white petrolatum, cetearyl alcohol and ceteareth-20, sorbitol solution, propylene glycol, simethicone, glyceryl monostearate, polyethylene glycol monostearate, sorbic acid, and butylated hydroxytoluene (BHT).

The compositions or carriers may be a transdermal gel such as Pluronic Lecithin Organogel (PLO). See Murdan, A Review of Pluronic Lecithin Organogel as a Topical and Transdermal Drug Delivery System, Hospital Pharmacist, July/August 2005, Vol. 12, pp. 267-270. Compositions using pluronic can range from 0.01% to 30% by weight of the composition of pluronic. Pluronic with or without a therapeutic agent such as spironolactone is injected into the meibomian gland via the meibomian gland orifice and serves as a stent to maintain an open lumen. The benefit would be to maintain a patient lumen to prevent the pathological process of fibrosis and further the chance of maintaining a lumen to prevent further loss of functional glands as well as promote healing and reestablishment of an open lumen and meibomian gland loss and function. These same compositions may be amenable for injection into the eye via the eye or surprachoroidal circulation for disease of the retina that have pathophysiologic associations with the ELOVL4 gene and/or Stargardt's disease and/or age-related macular degeneration.

In particular embodiments, the carrier is a polymer. Non-limiting examples of acceptable polymers include, hydroxyethyl cellulose, hydroxypropyl methyl cellulose, carboxymethyl cellulose (e.g., cellulose, or Gum Cellulose), polyethylene oxide, dextrans, and the like.

In a more particular embodiment, the carrier is hydroxypropyl methyl cellulose (HPMC) (also referred to as hypromellose).

Additional Compounds:

The compositions and/or carriers provided herein may include one or more additional compounds, or be used contemporaneously (e.g., used separately but with the compositions and/or carriers described herein) with, one or more additional compounds. Additional compounds may include antibiotics, steroids, anti-inflammation agents, analgesics, surfactants, chelating agents, buffering agents, pH adjusting agents, adjuvants, or combinations thereof. The additional compounds can provide any purpose, so long as the additional compounds are suitable for use in a composition or carrier used on a subject. Beneficial purposes of additional compounds may include synergistic effects when combined with the active ingredients of the composition (i.e., a greater than additive effect), composition and/or carrier stabilization, enhanced delivery of the compositions to the subject, ease of formulating, and combinations thereof.

Antibiotics:

In some aspects, the compositions and/or carriers may further include at least one antibiotic. The antibiotic may be any antibiotic suitable for use in a subject, in particular a mammalian subject, and more particularly, in a human subject. Non-limiting examples of antibiotics that may be used with the compositions and/or carriers described herein include amikacin, gentamicin, kanamycin, neomycin, netilmicin, streptomycin, tobramycin, teicoplanin, vancomycin, azithromycin, clarithromycin, dirithromycin, erythromycin, roxithromycin, troleandomycin, amoxicillin, ampicillin, azlocillin, carbenicillin, clozacillin, dicloxacillin, flucozacillin, meziocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, oflazacin, trovafloxacin, mafenide, sulfacetamide, sulfamethizole, sulfasalazine, sulfisoxazole, trimethoprim, cotrimoxazole, demeclocycline, soxycycline, minocycline, oxytetracycline, tetracycline, vancomycin, and salts thereof, and the like. Additionally, the antibiotics may include any sulfone such as dapsone (diaminodiphenyl sulfone (DDS)) or any dapsone derivative, such as amino acid amides of dapsone (see Pochopin et al., International Journal of Pharmaceutics, 121(2):157-167 (1995)), PROMIN (sodium glucosulfone), DIASONE (sulfoxone sodium), SULPHETRONE (solapsone), PROMIZOLE (thiazolsulfone), PROMACETIN (acetosulfone) and the like. Additional sulfones have been described (see Doub, Medicinal Chem, 5:350-425 (1961)). In any embodiment of the methods described in this disclosure, a sulfone such as dapsone may be administered to an ocular region of a subject with at least one aldosterone antagonist, or isomer, salt, or solvate thereof. The sulfone and at least one aldosterone antagonist may be administered in the same composition or in separate compositions, and may be administered simultaneously or sequentially one to the other. In embodiments, dapsone can be present in the composition in an amount ranging from 0.0005 wt % to 10 wt %, such as from 0.05 wt % to 5 wt %, or from 0.1 wt % to 3 wt %, or from 0.5 wt % to 0.8 wt %, or from 0.7 wt % to 4 wt % based on the total weight of the composition. Dapsone can be present in the composition with an amount of aldosterone antagonist (such as spironolactone) ranging from about 0.0005 wt % to 10 wt %, such as from about 0.0005 wt % to 1 wt %, or from 0.005 wt % to 5 wt %, or from 0.05 wt % to 3 wt %, or from about 0.5 wt % to 2 wt %, or from 0.07 wt % to about 6 wt % based on the total weight of the composition.

In embodiments, the amount of aldosterone antagonist (such as at least one aldosterone antagonist, or isomer, salt, or solvate thereof selected from the group consisting of spironolactone, eplerenone, canrenone, prorenone, mexrenone, derivatives thereof, and combinations thereof) can range from above 0 mg/cc to 10 mg/cc, or from 0.0000025 mg/cc to 10 mg/cc, or from 0.000005 mg/cc to 10 mg/cc, 0.000005 mg/cc to 8 mg/cc, 0.000005 mg/cc to 6 mg/cc, 0.000005 mg/cc to 5 mg/cc, 0.000005 mg/cc to 4 mg/cc, 0.000005 mg/cc to 3 mg/cc, 0.000005 mg/cc to 2 mg/cc, 0.000005 mg/cc to 1 mg/cc, 0.000005 mg/cc to 0.5 mg/cc, 0.000005 mg/cc to 0.1 mg/cc, 0.000005 mg/cc to 0.05 mg/cc, 0.000005 mg/cc to 0.04 mg/cc, 0.000005 mg/cc to 0.03 mg/cc, 0.000005 mg/cc to 0.025 mg/cc, 0.000005 mg/cc to 0.02 mg/cc, 0.000005 mg/cc to 0.01 mg/cc, 0.000005 mg/cc to 0.009 mg/cc, 0.000005 mg/cc to 0.008 mg/cc, 0.000005 mg/cc to 0.007 mg/cc, 0.000005 mg/cc to 0.006 mg/cc, 0.000005 mg/cc up to 0.005 mg/cc, 0.000005 mg/cc to 0.004 mg/cc, 0.000005 mg/cc to 0.003 mg/cc, 0.000005 mg/cc to 0.0025 mg/cc, 0.000005 mg/cc to 0.002 mg/cc, 0.000005 mg/cc to 0.001 mg/cc, 0.000005 mg/cc to 0.0009 mg/cc, 0.000005 mg/cc to 0.0008 mg/cc, 0.000005 mg/cc to 0.0007 mg/cc, 0.000005 mg/cc to 0.0006 mg/cc, 0.000005 mg/cc to 0.0005 mg/cc, 0.000005 mg/cc to 0.0004 mg/cc, 0.000005 mg/cc to 0.0003 mg/cc, 0.000005 mg/cc to 0.0002 mg/cc, 0.000005 mg/cc to 0.00015 mg/cc, 0.000005 mg/cc to 0.0001 mg/cc, 0.000005 mg/cc to 0.00009 mg/cc, 0.000005 mg/cc to 0.00008 mg/cc, 0.000005 mg/cc to 0.00007 mg/cc, 0.000005 mg/cc to 0.00006 mg/cc, 0.000005 mg/cc to 0.00005 mg/cc, 0.000005 mg/cc to 0.00004 mg/cc, 0.000005 mg/cc to 0.00003 mg/cc, 0.000005 mg/cc to 0.000025 mg/cc, 0.000005 mg/cc to 0.00002 mg/cc, 0.000005 mg/cc to 0.000015 mg/cc, 0.000005 mg/cc to 0.00001 mg/cc, 0.000005 mg/cc to 0.000009 mg/cc, 0.000005 mg/cc to 0.000008 mg/cc, 0.000005 mg/cc to 0.000007 mg/cc, and 0.000005 mg/cc to 0.000006 mg/cc, or any ranges in between.

Still further, the amount of aldosterone antagonist (such as at least one aldosterone antagonist, or isomer, salt, or solvate thereof selected from the group consisting of spironolactone, eplerenone, canrenone, prorenone, mexrenone, derivatives thereof, and combinations thereof) can range from above 0 mg/cc and below 10 mg/cc, or below 9 mg/cc, or below 8 mg/cc, or below 7 mg/cc, or below 6 mg/cc, or below 5 mg/cc, or below 4 mg/cc, or below 3 mg/cc, or below 2 mg/cc, or below 1 mg/cc, or below 0.5 mg/cc, or below 0.1 mg/cc, or below 0.05 mg/cc, or below 0.04 mg/cc, or below 0.03 mg/cc, or below 0.025 mg/cc, or below 0.01 mg/cc, from above 0 mg/cc to 0.000005 mg/cc, or from above 0 mg/cc to 0.009 mg/cc, to 0.008 mg/cc, to 0.007 mg/cc, to 0.006 mg/cc, up to 0.005 mg/cc, to 0.004 mg/cc, to 0.003 mg/cc, to 0.002 mg/cc, to 0.001 mg/cc, to 0.0009 mg/cc, to 0.0008 mg/cc, to 0.0007 mg/cc, to 0.0006 mg/cc, to 0.0005 mg/cc, to 0.0004 mg/cc, to 0.0003 mg/cc, to 0.0002 mg/cc, to 0.00015 mg/cc, to 0.0001 mg/cc, to 0.00009 mg/cc, to 0.00008 mg/cc, to 0.00007 mg/cc, to 0.00006 mg/cc, to 0.00005 mg/cc, to 0.00004 mg/cc, to 0.00003 mg/cc, to 0.000025 mg/cc, to 0.00002 mg/cc, to 0.000015 mg/cc, to 0.00001 mg/cc, to 0.000009 mg/cc, to 0.000008 mg/cc, to 0.000007 mg/cc, to 0.000006 mg/cc, or from above 0.0000025 mg/cc to below 0.005 mg/cc, or from above 0.00001 mg/cc to below 0.0025 mg/cc.

Steroidal Compounds:

In another aspect, the compositions and/or carriers may further include at least one steroid. The steroid may be any steroid suitable for use in a subject, in particular a mammalian subject, and more particularly, in a human subject. Non-limiting examples of steroids that may be used with the compositions and/or carriers described herein include 21-acetoxypregnenolone, acetonide, alclomethasone, algestone, amcinonide, beclomethasone, betamethasone, budesonide, chenodeoxycholic acid, chloroprednisone, clobetasol, clobetasone, clocortolone, cloprednol, corticosterone, cortisone, cortivazol, deflazacort, desonide, desoximethasone, dexamethasone, diflorasone diflucortolone, difluprednate, ethynylestradiol, estradiol, fluazacort, flucloronide, flumethasone, flunisolide, fluocinolone acetonide, fluocinonide, fluocortyn butyl, fluocortolone, fluorometholone, fluticasone propionate, fluperolone acetate, fluprednidene acetate, fluprednisolone, flurandrenolide, formocortal, halcinonide, halobetasol propionate, halomethasone, halopredone acetate, hexacetonide, hydrocortamate, hydrocortisone, loteprednol etabonate, mazipredone, medrysone, meprednisone, mestranol, methylprednisolone, mitatrienediol, momethasone furoate, moxestrol, paramethasone, prednicarbate, prednisolone, prednisolone 25-diethylaminoacetate, prednisolone sodium phosphate, prednisone, prednival, prednylidene, rimexolone, triamcinolone, triamcinolone, tixocortol, triamcinolone, ursodesoxycholic acid, and salts thereof, and the like.

Anti-Inflammatory Agents:

In yet other aspects, the compositions and/or carriers may further include at least one anti-inflammatory agent. The anti-inflammatory agent may be any anti-inflammatory agent suitable for use in a subject, in particular a mammalian subject, and more particularly, in a human subject. Non-limiting examples of anti-inflammatory agents include aceclofenac, acemetacin, acetylsalicylic acid, 5-amino-acetylsalicylic acid, alclofenac, alminoprofen, amfenac, bendazac, bermoprofen, α-bisabolol, bromfenac, bromosaligenin, bucloxic acid, butibufen, carprofen, cinmetacin, clidanac, clopirac, diclofenac sodium, diflunisal, ditazol, enfenamic acid, etodolac, etofenamate, felbinac, fenbufen, fenclozic acid, fendosal, fenoprofen, fentiazac, fepradinol, flufenamic acid, flunixin, flunoxaprofen, flurbiprofen, glucametacin, glycol salicylate, ibuprofen, ibuproxam, indomethacin, indoprofen, isofezolac, isoxepac, isoxicam, ketoprofen, ketorolac, lornoxicam, loxoprofen, meclofenamic acid, mefenamic acid, meloxicam, mesalamine, metiazinic acid, mofezolac, naproxen, niflumic acid, oxaceprol, oxaprozin, oxyphenbutazone, parsalmide, perisoxal, phenyl acetylsalicylate, olsalazine, pyrazolac, piroxicam, pirprofen, pranoprofen, protizinic acid, salacetamide, salicilamide O-acetic acid, salicylsulphuric acid, salsalate, sulindac, suprofen, suxibuzone, tenoxicam, tiaprofenic acid, tiaramide, tinoridine, tolfenamic acid, tolmetin, tropesin, xenbucin, ximoprofen, zaltoprofen, zomepirac, tomoxiprol or sulindac, salts thereof, and the like.

Analgesics:

In yet other aspects, the compositions and/or carriers may further include at least one analgesic. The analgesic may be any analgesic suitable for use in a subject, in particular a mammalian subject, and more particularly, in a human subject. Non-limiting examples of analgesics include acetaminophen (i.e., paracetamol), acetaminosalol, aminochlorthenoxazin, acetylsalicylic 2-amino-4-picoline acid, acetylsalicylsalicylic acid, anileridine, benoxaprofen, benzylmorphine, 5-bromosalicylic acetate acid, bucetin, buprenorphine, butorphanol, capsaicine, cinchophen, ciramadol, clometacin, clonixin, codeine, desomorphine, dezocine, dihydrocodeine, dihydromorphine, dimepheptanol, dipyrocetyl, eptazocine, ethoxazene, ethylmorphine, eugenol, floctafenine, fosfosal, glafenine, hydrocodone, hydromorphone, hydroxypethidine, ibufenac, p-lactophenetide, levorphanol, meptazinol, metazocine, metopon, morphine, nalbuphine, nicomorphine, norlevorphanol, normorphine, oxycodone, oxymorphone, pentazocine, phenazocine, phenocoll, phenoperidine, phenylbutazone, phenylsalicylate, phenylramidol, salicin, salicylamide, tiorphan, tramadol, diacerein, actarit or salts thereof, and the like.

Surf Actants/Wetting Agents:

In some aspects, the compositions and/or carriers may also include at least one surfactant or wetting agent. The surfactant may be selected from, but is not limited to, anionic, cationic, amphoteric, zwitterionic, and nonionic surfactants. If the surfactant is nonionic, it may be selected from the group consisting of polysorbates, poloxamers, alcohol ethoxylates, ethylene glycol-propylene glycol block copolymers, fatty acid amides, alkylphenol ethoxylates, or phospholipids, and the like. In particular, nonionic surfactants may include one or more poloxamer, such as one or more pluronic poloxamer, including but not limited to pluronic lecithin organogel, or pluronic can be provided by pluronic P-123, pluronic F-127, pluronic P-85, and/or pluronic F-68.

Chelating Agents:

In still other aspects, the compositions and/or carriers may also include a chelating agent, including but not limited to, edetate salts, like edetate disodium, edetate calcium disodium, edetate sodium, edetate trisodium, edetate dipotassium, and the like.

Buffering Agents:

In yet other aspects, the compositions and/or carriers may also include at least one buffer. Non-limiting examples of buffers may include phosphates (e.g., sodium phosphate, sodium dihydrogen phosphate, disodium hydrogen phosphate, potassium phosphate, potassium dihydrogen phosphate and dipotassium hydrogen phosphate, etc.), borates (e.g., sodium borate, potassium borate, etc.) citrates (e.g., sodium citrate, disodium citrate, etc.), acetates (e.g., sodium acetate, potassium acetate, etc.) carbonates (e.g., sodium carbonate, sodium hydrogen carbonate, etc.), and the like. Examples of organic buffers include HEPES, TES, BES, MES, MOPS, or PIPES. In some embodiments, the buffering agents maintain the pH of the composition at 4.75 to 8.0. In other embodiments, the buffering agents maintain the pH of the composition at 5.0-7.5 or 6.0-8.0. In other embodiments, the buffering agents maintain the pH of the composition at 5.2 to 7.2, or 5.5 to 6.8, or 7.0-8.0. In other embodiments, the buffering agents maintain the pH of the composition at 4.9 to 7.8, or 5.1 to 7.4, or 5.3 to 7.9, or 7.2-7.6. In other embodiments, the buffering agents maintain the pH of the composition at 7.4.

pH Adjusting Agents:

In yet still other aspects, the compositions and/or carriers may also include at least one pH adjusting agent. Non-limiting examples of pH adjusting agents include sodium hydroxide, potassium hydroxide, sodium carbonate, hydrochloric acid, phosphoric acid, citric acid, acetic acid, and the like.

Preservatives:

In still yet other aspects, the compositions and/or carriers may be preservative free or may also include at least one preservative. Non-limiting examples of preservatives include p-hydroxy benzoate esters, benzalkonium chloride, benzethonium chloride, esters of parahydroxybenzoates (parabens), organic mercurial compounds (phenylmercuric acetate, phenylmercuric nitrate and thimerosal), detergent (Polyquad), oxidizing agent (Purite), chlorobutanol, benzyl alcohol, phenylethylalcohol, sorbic acid or its salts, chlorhexidine gluconate, sodium dehydroacetate, cetylpyridinium chloride, alkyldiaminoethylglycine hydrochloride, and ethylenediaminetetraacetic acid (EDTA). Other compounds that may be included in the compositions and/or carriers can include oleic acid, 1-methyl-2 pyrrolidone, 2,2-dimethyl octanoic acid and N,N dimethyl lauramide/propylene glycol monolaureate or combinations thereof, which may be included for example to minimize the barrier characteristics of the upper most layer of the corneal and conjunctival surfaces, thus, improving efficacy.

Adjuvants:

Further still, the compositions or carriers provided herein, may also include one or more adjuvants. Non-limiting examples of suitable adjuvants include phosphatidic acid, sterols such as cholesterol, aliphatic amines such as stearylamine, saturated or unsaturated fatty acids such as stearic acid, palmitic acid, myristic acid, linoleic acid, oleic acid, and salts thereof, and the like.

Antioxidants:

Antioxidants may be added to optimize the stability of therapeutic agents that degrade by oxidation. Examples of such antioxidants include sodium metabisulphite, butylated hydroxytoluene, and butylated hydroxyanisole.

Methods

Further disclosed are methods for treating at least one ocular surface disease comprising administering to an ocular region of a subject one or more of the compositions described herein.

In another aspect, methods for treating at least one ocular surface disease comprise administering a composition comprising a pharmaceutically effective amount of at least one aldosterone antagonist (including isomers, salts, and solvates thereof) and a carrier to the ocular region of a subject.

In still a more particular aspect of the disclosed methods, the composition described herein is a composition that delivers at least one aldosterone antagonist (including isomers, salts, and solvates thereof) having a desired therapeutically effective amount of aldosterone antagonist in the range of about 0.00000025 wt. % to 1.00 wt. % of the composition or carrier to the ocular region of a subject to be treated. It is envisioned that the therapeutically effective amount of the at least one aldosterone antagonist could be greater than 1.00 wt. % depending what can be tolerated by the subject being treated and the clinical effect(s) at the site of action (ocular surface anatomical structures, including the cornea, conjunctiva, lid margin epithelium, blood vessels, the meibomian gland/sebaceous gland complex, ducts, lumens, orifices. etc.). As provided throughout, the aldosterone antagonists used to carry out the methods described herein can be any aldosterone antagonist or isomer, salt, or solvate thereof. In particular aspects, the aldosterone antagonist is spironolactone or an isomer, salt or solvate thereof.

In a particular aspect, the methods described herein treat front of the eye ocular surface diseases. In another aspect, the methods described herein treat back of the eye ocular surface diseases. In still another aspect, the methods described herein treat both front of the eye diseases and back of the eye diseases.

Non-limiting examples of front of the eye ocular surface diseases include inflammation, diffuse lamellar keratitis, corneal diseases, edemas, or opacifications with an exudative or inflammatory component, diseases of the eye that are related to systemic autoimmune diseases, any ocular surface disorders, keratoconjunctivitis, such as vernal keratoconjunctivitis, atopic keratoconjunctivitis, and sicca keratoconjunctivitis,), lid margin diseases, meibomian gland disease or dysfunction, dysfunctional tear syndromes, anterior and or posterior blepharitis, microbial infection, computer vision syndrome, conjunctivitis (e.g., persistent allergic, giant papillary, seasonal intermittent allergic, perennial allergic, toxic, conjunctivitis caused by infection by bacteria, fungi, parasites, viruses or Chlamydia), conjunctival edema anterior uveitis and any inflammatory components or components of the aqueous fluid, inflammatory conditions resulting from surgeries such as LASIK®, LASEK®, refractive surgery, intraocular lens implantation (IOL), irreversible corneal edema as a complication of cataract surgery, edema as a result of insult or trauma (physical, chemical, pharmacological, etc), genetic diseases of the cornea (corneal dystrophies including keratoconus, posterior polymorphous dystrophy; Fuch's dystrophies (corneal and endothelial), etc.), aphakic and pseudophakic bullous keratopathy, scleral diseases with or without inflammatory components, ocular cicatricial pemphigoid, pterygium, and the like.

Non-limiting examples of back of the eye diseases include diseases of the optic nerve (including its cellular and sub cellular components such as the axons and their innervations), glaucomas (including primary open angle glaucoma, acute and chronic closed angle glaucoma and any other secondary glaucomas), myopic retinopathies, macular edema (including clinical macular edema or angiographic cystoid macular edema arising from various etiologies such as diabetes, exudative macular degeneration and macular edema arising from laser treatment of the retina), diabetic retinopathy, age-related macular degeneration, retinopathy of prematurity, retinal ischemia and choroidal neovascularization and like diseases of the retina, genetic disease of the retina, e.g., Stargardt's macular dystrophy and age-related macular degeneration, pars planitis, Posner Schlossman syndrome, Bechet's disease, Vogt-Koyanagi-Harada syndrome, hypersensitivity reactions, toxoplasmosis chorioretinitis, inflammatory pseudotumor of the orbit, chemosis, conjunctival venous congestion, periorbital cellulitis, acute dacryocystitis, non-specific vasculitis, sarcoidosis, cytomegalovirus infection, and the like.

In particular aspects, the methods described herein treat ocular surface disease (including ADDE, EDE, chronic dry eye, MGD, etc.).

In embodiments, compositions described herein are administered to the ocular region of a subject by topical administration. In one aspect, there is provided a method for treating ocular surface disease which includes the ocular or lid region, including treatment of the eyelid for MGD, the method comprising: topically administering to an ocular region of a subject a composition comprising spironolactone and hydroxypropyl methylcellulose, and/or optionally comprising one or more preservatives, and/or optionally comprising one or more compounds for increasing efficacy; and reducing or preventing one or more symptoms or causes of ocular surface disease. In embodiments the spironolactone of the compositions can be substituted with or supplemented with one or more of eplerenone, canrenone, prorenone, and/or mexrenone.

In still other embodiments, the compositions are administered with one or more additional pharmaceutical agents. The one or more additional pharmaceutical agents may be administered, before, after, or simultaneously with the administration of the compositions described herein. In one aspect, the one or more additional pharmaceutical agents is administered before the administration of the compositions described herein. In another aspect, the one or more additional pharmaceutical agents is administered after the compositions described herein. In still yet another aspect, the one or more additional pharmaceutical agents is administered simultaneously with the administration of the compositions described herein. In embodiments where the one or more additional pharmaceutical agents is administered simultaneously with the administration of the compositions, the additional pharmaceutical agent may be formulated with the compositions described herein or administered as a separate pharmaceutical agent at about the same time as the compositions described herein are administered.

Such methods can comprise administering a composition comprising from 0 to 10%, such as from between 0.000005 mg/cc to 0.009 mg/cc, or from between 0.05% and 10%, such as from between 0.05% and 1%, or from between 0.1% and 1%, or from between 0.15% and 0.8%, or from between 0.2% and 0.7%, or from between 0.3% and 0.5%, or from between 0.4% and 0.9% aldosterone antagonist, such as spironolactone, eplerenone, canrenone, prorenone, and/or mexrenone (based on weight or volume of the composition). In embodiments, the amount of aldosterone antagonist (such as at least one aldosterone antagonist, or isomer, salt, or solvate thereof selected from the group consisting of spironolactone, eplerenone, canrenone, prorenone, mexrenone, derivatives thereof, and combinations thereof) in the composition(s) administered can range from above 0 mg/cc to 10 mg/cc, or from 0.0000025 mg/cc to 10 mg/cc, or from 0.000005 mg/cc to 10 mg/cc, 0.000005 mg/cc to 8 mg/cc, 0.000005 mg/cc to 6 mg/cc, 0.000005 mg/cc to 5 mg/cc, 0.000005 mg/cc to 4 mg/cc, 0.000005 mg/cc to 3 mg/cc, 0.000005 mg/cc to 2 mg/cc, 0.000005 mg/cc to 1 mg/cc, 0.000005 mg/cc to 0.5 mg/cc, 0.000005 mg/cc to 0.1 mg/cc, 0.000005 mg/cc to 0.05 mg/cc, 0.000005 mg/cc to 0.04 mg/cc, 0.000005 mg/cc to 0.03 mg/cc, 0.000005 mg/cc to 0.025 mg/cc, 0.000005 mg/cc to 0.02 mg/cc, 0.000005 mg/cc to 0.01 mg/cc, 0.000005 mg/cc to 0.009 mg/cc, 0.000005 mg/cc to 0.008 mg/cc, 0.000005 mg/cc to 0.007 mg/cc, 0.000005 mg/cc to 0.006 mg/cc, 0.000005 mg/cc up to 0.005 mg/cc, 0.000005 mg/cc to 0.004 mg/cc, 0.000005 mg/cc to 0.003 mg/cc, 0.000005 mg/cc to 0.0025 mg/cc, 0.000005 mg/cc to 0.002 mg/cc, 0.000005 mg/cc to 0.001 mg/cc, 0.000005 mg/cc to 0.0009 mg/cc, 0.000005 mg/cc to 0.0008 mg/cc, 0.000005 mg/cc to 0.0007 mg/cc, 0.000005 mg/cc to 0.0006 mg/cc, 0.000005 mg/cc to 0.0005 mg/cc, 0.000005 mg/cc to 0.0004 mg/cc, 0.000005 mg/cc to 0.0003 mg/cc, 0.000005 mg/cc to 0.0002 mg/cc, 0.000005 mg/cc to 0.00015 mg/cc, 0.000005 mg/cc to 0.0001 mg/cc, 0.000005 mg/cc to 0.00009 mg/cc, 0.000005 mg/cc to 0.00008 mg/cc, 0.000005 mg/cc to 0.00007 mg/cc, 0.000005 mg/cc to 0.00006 mg/cc, 0.000005 mg/cc to 0.00005 mg/cc, 0.000005 mg/cc to 0.00004 mg/cc, 0.000005 mg/cc to 0.00003 mg/cc, 0.000005 mg/cc to 0.000025 mg/cc, 0.000005 mg/cc to 0.00002 mg/cc, 0.000005 mg/cc to 0.000015 mg/cc, 0.000005 mg/cc to 0.00001 mg/cc, 0.000005 mg/cc to 0.000009 mg/cc, 0.000005 mg/cc to 0.000008 mg/cc, 0.000005 mg/cc to 0.000007 mg/cc, and 0.000005 mg/cc to 0.000006 mg/cc, or any ranges in between.

Still further, the amount of aldosterone antagonist (such as at least one aldosterone antagonist, or isomer, salt, or solvate thereof selected from the group consisting of spironolactone, eplerenone, canrenone, prorenone, mexrenone, derivatives thereof, and combinations thereof) in the composition(s) administered can range from above 0 mg/cc and below 10 mg/cc, or below 9 mg/cc, or below 8 mg/cc, or below 7 mg/cc, or below 6 mg/cc, or below 5 mg/cc, or below 4 mg/cc, or below 3 mg/cc, or below 2 mg/cc, or below 1 mg/cc, or below 0.5 mg/cc, or below 0.1 mg/cc, or below 0.05 mg/cc, or below 0.04 mg/cc, or below 0.03 mg/cc, or below 0.025 mg/cc, or below 0.01 mg/cc, from above 0 mg/cc to 0.000005 mg/cc, or from above 0 mg/cc to 0.009 mg/cc, to 0.008 mg/cc, to 0.007 mg/cc, to 0.006 mg/cc, up to 0.005 mg/cc, to 0.004 mg/cc, to 0.003 mg/cc, to 0.002 mg/cc, to 0.001 mg/cc, to 0.0009 mg/cc, to 0.0008 mg/cc, to 0.0007 mg/cc, to 0.0006 mg/cc, to 0.0005 mg/cc, to 0.0004 mg/cc, to 0.0003 mg/cc, to 0.0002 mg/cc, to 0.00015 mg/cc, to 0.0001 mg/cc, to 0.00009 mg/cc, to 0.00008 mg/cc, to 0.00007 mg/cc, to 0.00006 mg/cc, to 0.00005 mg/cc, to 0.00004 mg/cc, to 0.00003 mg/cc, to 0.000025 mg/cc, to 0.00002 mg/cc, to 0.000015 mg/cc, to 0.00001 mg/cc, to 0.000009 mg/cc, to 0.000008 mg/cc, to 0.000007 mg/cc, to 0.000006 mg/cc, or from above 0.00001 mg/cc to below 0.005 mg/cc, or from above 0.00001 mg/cc to below 0.0025 mg/cc.

Such methods can include administering the composition(s) from between 1-8 times daily; and/or from between 1-4 times daily, for 1-4 weeks; and/or from between 1-4 times daily, for up to 4 weeks, then from 1-2 times daily. The composition(s) can be administered at any frequency, such as any number of times per day, every other day, or every 2-3 days, or weekly or monthly. The composition(s) can be administered for any period of time as well, such as for up to 1 day, from 1-7 days, or for up to 1 week, for up to 30 days, or up to 60 days, or up to 90 days, or up to 120 days, or from 1-52 weeks, or for up to 1 year, or from 1-20 years, or indefinitely. Methods of administering can include administering a first formulation for a selected period of time and frequency, then administering a second formulation for a selected period of time and frequency. For example, a first composition with a certain amount of active agent, such as one or more aldosterone antagonist, like spironolactone, can be administered to a patient for a selected period of time and frequency. Then a second composition with an amount of active agent that is lower or higher or the same as that of the first composition, and/or in the same or different form (e.g., suspension, solution, injectable, etc.) can be administered to the patient for a selected period of time and frequency, where the time and/or frequency for administering the second composition can be the same or different. In embodiments, for example, a suspension of an effective amount of active agent could be administered to a patient as the first composition, then a solution of an effective amount of active agent could be administered as the second composition. Further, for example, the first composition administered can have an amount of active agent at one amount, while the second composition administered can have an amount of active agent at the same or a different amount, such as a higher or lower amount. For some patients, it may be expected that the compositions can be administered indefinitely, permanently, or otherwise on a long-term basis as a maintenance therapy. In some, instances, the method can include administering the compositions described herein to a subject throughout the lifetime of the subject as a maintenance therapy.

The method can be used to prevent and/or reduce one or more symptoms and/or causes of ocular surface disease which includes the ocular or lid region, including treatment of the eyelid for MGD, such as impaired vision, burning sensation, redness, irritation, grittiness, filminess, inflammation, discomfort, pain, chemosis, chalasis, engorged vasculature, anterior lid margin vascularization, Zone A posterior lid margin vascularization, or meibomian gland obstruction, secretion, viscosity, turbidity, loss, drop out, or dysfunction. According to methods of the invention, the reducing or preventing of symptoms or causes of ocular surface disease is quantitatively or qualitatively evidenced by vital staining, such as by lissamine green staining.

In an aspect, the compositions described herein are topically administered to the eye to treat ocular surface disease, which includes the ocular or lid region, including treatment of the eyelid for MGD. In a particular aspect, the compositions described herein are topically administered to the cornea to treat ocular surface disease. In still another particular aspect, the compositions described herein are topically administered to the sclera to treat ocular surface disease. In still yet another particular aspect, the compositions described herein are topically administered to the conjunctiva to treat ocular surface disease. In yet still another particular aspect, the compositions described herein are topically administered to the lacrimal sac to treat ocular surface disease. In another particular aspect, the compositions described herein are topically administered to the lacrimal canals to treat ocular surface disease. In still another particular aspect, the compositions described herein are topically administered to the lacrimal ducts to treat ocular surface disease. In yet another particular aspect, the compositions described herein are topically administered to the canthus to treat ocular surface disease. In still yet another particular aspect, the compositions described herein are topically administered to the eyelids to treat ocular surface disease.

In one aspect, the compositions described herein are topically administered by administering a liquid or a microsuspension, such as an imperceivable microsuspension solution (e.g., ophthalmic drops) to the ocular region of a subject for example to treat ocular surface disease, which includes treating the ocular or lid region, including treatment of the eyelid for MGD. Not wishing to be bound by theory, the core mechanism of MGD is an obstructive process that is caused by hyperkeratinization of the meibomian duct and orifice, together with increased viscosity of meibum. Hyperkeratinization appears to be the main pathological mechanism of MGD. This hyperkeratinization results in narrowing of the MG orifices and a loss of functional glandular tissue. The increased viscosity of meibum results in increased stasis inside the ductal system, contributing to the obstructive process. Further, alterations in the lipid composition of meibum may contribute to an increase in tear film instability and evaporation in patients with MGD. Associated processes with MGD include altered cell differentiation, seborrhea, commensal bacterial growth, and the production of inflammatory mediators. For a detailed review of the pathophysiology of MGD, see E. Knop et al., “The International Workshop on Meibomian Gland Dysfunction: Report of the Subcommittee on Anatomy, Physiology, and Pathophysiology of the Meibomian Gland”, Invest Ophthalmol Vis Sci. 2011 March; 52(4): 1938-1978, incorporated by reference herein in its entirety.

In yet another particular aspect, the compositions described herein are topically administered by administering a suspension to the ocular region of a subject for example to treat ocular surface disease which includes the ocular or lid region, including treatment of the eyelid for MGD. In another particular aspect, the compositions described herein are topically administered by administering a cream to the ocular region of a subject for example to treat ocular surface disease. In still another particular aspect, the compositions described herein are topically administered by administering an emulsion to the ocular region of a subject for example to treat ocular surface disease. In yet another particular aspect, the compositions described herein are topically administered by administering a gel to the ocular region of a subject for example to treat ocular surface disease. In still yet another particular aspect, the compositions described herein are topically administered by administering a paste, pellet, ointment, spray, or nanoparticle vehicle to the ocular region of a subject for example to treat ocular surface disease. In preferred embodiments, the composition comprises xanthan gum. In yet still another particular aspect, the compositions described herein are topically administered by administering a gel to the ocular region of a subject for example to treat ocular surface disease. In another particular aspect, the compositions described herein are topically administered by administering an ointment to the ocular region of a subject for example to treat ocular surface disease. In still another particular aspect, the compositions described herein are topically administered by administering a particle (e.g., a nanosized or macrosized particle, pellet, etc.) to the ocular region of a subject for example to treat ocular surface disease. In yet another particular aspect, the compositions described herein are topically administered by administering a slurry to the ocular region of a subject for example to treat ocular surface disease.

The administering step can be performed by any method known in the art (e.g., liquid dropper, injection, nanoparticle vehicles, gum materials (e.g., xanthan gum materials, sprays, application of the compositions described herein to a material worn over the eye, such as a patch, contact lenses, etc.). Various drug forms and methods for topical ocular administration have been reviewed, and each are contemplated for use with the invention (see P. Baranowski et al., “Ophthalmic Drug Dosage Forms: Characterization and Research Methods”, The Scientific World Journal Volume 2014 (2014), Article ID 861904, http://dx.doi.org/10.1155/2014/861904, 14 pages; A Patel, “Ocular drug delivery systems: An overview”, World J Pharmacol. 2013; 2(2): 47-64; each incorporated by reference herein in their entireties). The step of administering the compositions may be repeated as necessary (e.g., more than once, as in the administering step is repeated twice, three times, four times, five times, six times, seven times, eight times, nine times, ten times, eleven times, twelve times, thirteen times, fourteen times, fifteen times, sixteen times, seventeen times, eighteen times, nineteen times, twenty times, etc.) until the ocular surface disease is treated.

The aldosterone antagonist, alone or in combination with other active agents, such as dapsone and/or prednisone, or a preparation comprising these components, can be injected subconjunctivally as well as subtarsally into the eye lids and/or meibomian glands directly and/or into the ducts of the glands directly.

### EXAMPLES

The following examples are provided for illustrative purposes and are not intended to limit the scope of the embodiments as claimed herein. Any variations in the exemplified examples which occur to the skilled artisan are intended to fall within the scope of the present disclosure.

### Example 1

### Preparation of the Composition

Materials:

Aldosterone Antagonist:

Spironolactone powder (Letco Medical, Decatur, Ala., USA) or (PCCA, Houston, Tex., USA) (or an equivalent amount of eplerenone, canrenone, prorenone, and/or mexrenone, or combinations with spironolactone).

Carrier:

Hypromellose—PF (preservative free 0.3% solution of Hypromellose without sodium chloride; buffered with sodium phosphate) (Prepared by Greenpark Pharmacy of Houston, Tex.).

Methods:

In a glass mortar with pestle, wet the spironolactone with drops of hypromellose (HPMC) until a paste is made. Preferably, the HPMC starting material for mixing with the aldosterone agent comprises from 0.01% to 5% HPMC, such as from 0.05% to 0.8%, or from 0.1% to 0.5%, or from 0.2% to 1%, or from 0.3% to 2%, or from 0.4% to 3%, or from 0.5% to 4%, and is preservative free. Continue to gradually add the hypromellose until a total amount of approximately 90% to 99.9% by weight of the total composition is the HPMC starting material and is mixed to make a suspension of the spironolactone. Preferably, in the finished product, approximately 91% to 99.8% HPMC starting material is used (percent by weight of the total composition), such as from about 92% to 99.7%, or from about 93% to 99.6%, or from about 94% to 99.5%, or from about 95% to 99.4%, or from about 96% to 99.3%, or from about 97% to 99.2%, or from about 98% to 99.1%, or about 99%. Transfer the suspension into proper size amber glass vial with a spin bar. Cap and seal the vial. Ensure proper size vial is selected to leave enough head space in top of the vial to prevent the cap from coming off during autoclaving. Autoclave the vial with the contents. Immediately after autoclave, place vial onto a hot plate spin bar stirrer. Spin bar stir the suspension over night at room temperature. Transfer 15 ml into each drop container. Sterilization procedures can be used in addition to or alternatively to autoclaving. Any known sterilization procedure or combinations of such procedures can be used. Discard any remaining contents 30 days after opening. (preservative free).

### Example 2

### Administering the Composition to a Subject

A composition of Example 1 is administered to a number of subjects. The subjects are instructed to administer the composition of Example 1 to the eye up to four-times a day using ophthalmic drops for 1-4 weeks.

Results indicate that after two weeks of treatment using the composition of Example 1 as instructed, the subjects are reporting less redness, less irritation, less grittiness, and greater tolerance for their symptoms.

Quantitative results indicate that patients using the composition of Example 1 as instructed tend to have less conjunctival redness, improved obstruction of the meibomian glands, and/or improved turbidity of the glands. Quantitative results can be obtained using any vital staining technique, including for example lissamine green staining, rose Bengal staining, and/or sodium fluorescein staining. Such staining techniques can be used to identify and/or quantify a degree of epithelial cellular disruption, for example by staining dead and degenerate cells while not staining healthy cells. Treated patients/subjects may also exhibit improved keratitis scores.

### Example 3

### Pilot Study

Background:

Conventional treatments for dry eye syndrome which includes the ocular or lid region, including treatment of the eyelid for MGD have focused on addressing tear levels and inflammation, but have failed to demonstrate efficacy in all patients. New therapies have increasingly addressed meibomian gland dysfunction (MGD). Topical spironolactone is a drug with low toxicity and the potential to regulate and improve sebaceous gland meibum secretions through a variety of mechanisms.

Purpose:

The objective of this study was to investigate the effectiveness of topical spironolactone in treating MGD, a major component of dry eye syndrome.

Design:

Retrospective cohort study.

Methods Setting: Clinical practice.

Patient Study Population: Twenty patients from November 2014 to February 2015 with moderate to severe meibomian gland disease were included in this study. The prescribing information included administering a composition to both eyes of the subjects 4 times per day as a topical drop for one month and then 2 times per day henceforth for maintenance. Any formulation in this disclosure can be administered according to any protocol provided herein as well, or according to typical treatment protocols. Patients who were taking glaucoma medications, steroid eye drops and other lipid-altering eye drops prior to starting spironolactone were excluded. Several parameters were analyzed in describing MGD, including subjective global dry eye assessment, keratitis and conjunctival staining, anterior blepharitis grade, gland obstruction grade, meibum turbidity grade, meibum viscosity grade, Zone A posterior lid margin grade, best corrected vision, and Schirmer's score. These parameters were compared in a pre-post study. Follow-up times ranged from 1 to 7 weeks, with an average of approximately 3 weeks. Main outcome measures: Parameters in the prevalence of meibomian gland dysfunction (subjective global assessment, lissamine green keratitis and conjunctival staining, anterior blepharitis grade, obstruction grade, vascularity grade, turbidity grade, Zone A grade, vision and Schirmer's tear score) in patients with moderate to severe MGD. Zone A is the region of the posterior lid margin approximately 1 mm behind the posterior lid margin that is typically avascular. When vascularized, it suggests MGD or chronic inflammation.

Results:

Patients with moderate to severe MGD had improved self-reported global assessment scores (p=0.010), turbidity score (p=0.001), and Zone A scores (p=0.025) after treatment with topical ophthalmic suspensions of spironolactone.

Conclusions:

MGD patients reported improved symptoms after using compounded topical spironolactone ophthalmic suspensions for longer than one week. The quality of expressed meibum secretions of MGD patients clinically showed improved clarity and viscosity post-treatment. Inflammation decreased at the avascular region ˜0.5 mm posterior to the posterior lid margin post-treatment. This study demonstrates the potential for spironolactone to regulate meibum quality and address inflammation in treating MGD.

Spironolactone has potential to be used to treat MGD due to its pharmacological properties. It is believed that spironolactone addresses oil production in MGD by modulating testosterone receptors, and address inflammation associated with MGD by suppressing production of cytokines and cortisol. Patients taking spironolactone had an improvement in subjective dry-eye symptoms, turbidity scores (quality of expressed meibum), and Zone A scores (posterior lid margin inflammation).

Detailed Methods:

This study is a retrospective chart review of 20 patients, 12 female and 8 male with a mean age of 48.3±18.4 years. Corrected visual acuity was measured prior to starting spironolactone and during the follow-up visit. Corrected visual acuity was recorded in log MAR based on the corresponding line read correctly on the Snellen chart. Patients self-assessed dry-eye global scores based on the presence of symptoms during the current exam on a scale from 0 to 10 based on (0=no dry eye symptoms, 10=the worst dry eye symptoms felt ever). Keratitis and conjunctival scores were evaluated using lissamine green staining on nasal conjunctival, central corneal, and temporal conjunctival regions on a scale from 0 to 3 (0=no staining and 3=confluent staining). Schirmer's test was performed without topical anesthetics to evaluate tear film production. Anterior blepharitis scores were evaluated using a slit lamp and graded on a scale from 0 to 4 (0=no dandruff and 4=dandruff across the entire lash line). Lid margin abnormalities such as vascularity and inflammation of the avascular region ˜0.5 mm posterior to the posterior lid margin (Zone A) were evaluated and graded on a scale from 0 to 4 (0=no vascularization and 4=vascularization of the entire margin). See Arita, R., Zavala, M., & Yee, R. W., “MGD Diagnosis,” Curr Opthalmol Rep, 49-57 (Jun. 4, 2014). meibomian gland expression (obstruction) was evaluated by applying pressure to the lower lid and graded on a scale from 0 to 4. The quality of the expressed meibum (turbidity and viscosity) was also evaluated and graded from 0 to 4 (0=clear oil; 4=cloudy & toothpaste-like). The diagnosis of MGD was made based on the presence of a score of 3+ on symptoms or 2+ on lid margin abnormalities (see Arita et al., “Proposed diagnostic criteria for obstructive meibomian gland dysfunction,” Ophthalmology, 116:2058-2063 (2009)). Patients taking glaucoma medications or steroid eye-drops were excluded from the study. Patients included in the study started using spironolactone eye drops prior to using other topical eye medications or systemic medications to treat any dry eye conditions. Patients were prescribed spironolactone after previously taking omega-3 fatty acid and flax seed oil supplements and practicing blinking exercises with limited improvement. The average follow-up interval in this pre-post study was 22.4 days, ranging from 2 to 6 weeks. Statistical analysis was performed using STATA 13 by fitting scored data to a non-parametric model with the Wilcoxon signed-rank test and testing continuous data with a paired t-test.

Detailed Results:

The mean subjective global assessment score of MGD prior to treatment based on self-reported symptoms was 6.0±0.610. After treatment with spironolactone, the mean subjective global assessment score was 4.6±0.534. The patients had an improvement of 1.4±0.597 (p=0.0113) in self-reported global assessment scores. The mean keratitis and conjunctival staining scores of the right eye prior to treatment were 1.0±0.201, 0.10±0.100, and 0.78±0.194 in the nasal, corneal, and temporal regions respectively. The mean keratitis and conjunctival staining scores of the left eye prior to treatment were 0.95±0.198, 0.20±0.138, and 0.30±0.164 in the nasal, corneal, and temporal regions respectively. Post-treatment mean keratitis and conjunctival scores were 0.83±0.189, 0.050±0.050 and 0.55±0.181, in nasal, corneal, and temporal regions of the right eye and 0.60±0.148, 0.20±0.156 and 0.40±0.134 in nasal, corneal, and temporal regions of the left eye, respectively. The mean change of keratitis and conjunctival scores were −0.20±0.142 (p=0.168), −0.05±0.114 (p=0.655), and −0.23±0.187 (p=0.293) in nasal, corneal, and temporal regions of the right eye and −0.35±0.146 (p=0.037), 0.00±0.126 (p=1.00), 0.10±0.204 (p=0.366) in nasal, corneal, and temporal regions of the left eye.

The mean visions of patients prior to treatment were 0.072±0.036 and 0.022±0.034 log Mar units in the left and right eyes, respectively. After treatment, the vision of patients was 0.063±0.032 and 0.069±0.051 log units in the left and right eyes, respectively. The mean change in MAR was −0.0088±0.032 log units (p=0.999) for the left eye and 0.047±0.043 log units (p=0.236) for the right eye.

The mean Schirmer's scores of patients prior to treatment were 14.15±2.171 mm and 14.30±2.306 mm for the left and right eyes, respectively. Post treatment, the mean Schirmer's scores were 13.63±2.421 mm and 14.55±2.426 mm for the left and right eyes. The mean change in Schirmer's score was 0.250±1.008 and −0.525±1.496 for the left and right eyes, respectively.

The mean anterior blepharitis score was 0.55±0.226 prior to treatment and 0.38±0.114 post-treatment; mean improvement in anterior blepharitis score was 0.175±0.230 (p=0.8985). The mean vascularity score was 1.73±0.264 prior to treatment and 1.48±0.253 post-treatment, with a mean improvement of 0.25±0.194 (p=0.2699). The mean obstruction score was 1.98±0.234 prior to treatment and 1.58±0.236 post-treatment, with a mean improvement of 0.40±0.222 (p=0.1114). The mean turbidity score was 2.95±0.125 prior to treatment and 2.3±0.193 post-treatment, with a mean improvement of 0.65±0.141 (p=0.0010), corresponding to an improvement of 22.03%. The mean zone A score was 3.55±0.149 prior to treatment and 3.125±0.188 post-treatment, with a mean improvement of 0.425±0.186 (p=0.0248), corresponding to an improvement of 11.97%. The pre-treatment and post-treatment measurements, and change between pre- and post-treatment, are summarized below in Tables I-III.

TABLE I
Pre-treatment Descriptive Statistics

| | | Standard |
|---|---|---|
| Parameter | Mean | Deviation |
| Subjective Global Assessment | 6.0 | 2.73 |
| Conjunctival OD (Nasal) | 1.0 | 0.896 |
| Keratitis OD (Corneal) | 0.10 | 0.447 |
| Conjunctival OD (Temporal) | 0.78 | 0.866 |
| Conjunctival OS (Nasal) | 0.95 | 0.887 |
| Keratitis OS (Corneal) | 0.20 | 0.616 |
| Conjunctival OS (Temporal) | 0.30 | 0.733 |
| Vision OD (log Mar units)* n = 19 | 0.022 | 0.149 |
| Vision OS (log Mar units) | 0.072 | 0.160 |
| Schirmer's OD (mm) | 14.2 | 9.71 |
| Schirmer's OS (mm) | 14.3 | 10.3 |
| Anterior Blepharitis | 0.55 | 1.01 |
| Vascularity | 1.73 | 1.18 |
| Obstruction | 1.98 | 1.04 |
| Turbidity | 2.95 | 0.560 |
| Zone A | 3.55 | 0.667 |

*One patient was excluded from vision since he had prosthesis in his right eye

TABLE II
Post-treatment Descriptive Statistics

| | | Standard |
|---|---|---|
| Parameter | Mean | Deviation |
| Subjective Global Assessment | 4.6 | 2.39 |
| Conjunctival OD (Nasal) | 0.83 | 0.847 |
| Keratitis OD (Corneal) | 0.050 | 0.224 |
| Conjunctival OD (Temporal) | 0.55 | 0.809 |
| Conjunctival OS (Nasal) | 0.60 | 0.661 |
| Keratitis OS (Corneal) | 0.20 | 0.696 |
| Conjunctival OS (Temporal) | 0.40 | 0.598 |
| Vision OD (log Mar units)* n = 19 | 0.069 | 0.221 |
| Vision OS (log Mar units) | 0.063 | 0.141 |
| Schirmer's OD (mm) | 13.6 | 10.8 |
| Schirmer's OS (mm) | 14.6 | 10.9 |
| Anterior Blepharitis | 0.38 | 0.510 |
| Vascularity | 1.48 | 1.13 |
| Obstruction | 1.58 | 1.05 |
| Turbidity | 2.3 | 0.865 |
| Zone A | 3.125 | 0.841 |

*One patient was excluded from vision since he had prosthesis in his right eye

TABLE III
Pre-post change

| Parameter | Mean Change | Change % | SEM | p-value |
|---|---|---|---|---|
| Subjective Global | −1.4 | −23.33% | 2.67 | 0.0113 |
| Assessment | | | | |
| Conjunctival OD (Nasal) | −0.20 | −20.00% | 0.637 | 0.168 |
| Keratitis OD (Corneal) | −0.05 | −50.00% | 0.510 | 0.655 |
| Conjunctival OD | −0.23 | −29.49% | 0.835 | 0.293 |
| (Temporal) | | | | |
| Conjunctival OS (Nasal) | −0.35 | −36.84% | 0.651 | 0.037 |
| Keratitis OS (Corneal) | 0.00 | 0.00% | 0.562 | 1.00 |
| Conjunctival OS | 0.10 | 20.00% | 0.912 | 0.366 |
| (Temporal) | | | | |
| Vision OD (log Mar units)* | 0.047 | 213.64% | 0.189 | 0.236 |
| n = 19 | | | | |
| Vision OS (log Mar units) | 0.009 | −7.20% | 0.144 | 0.999 |
| Schirmer's OD (mm) | −0.525 | −3.71% | 6.69 | 0.7295 |
| Schirmer's OS (mm) | 0.25 | 1.75% | 4.51 | 0.8067 |
| Anterior Blepharitis | −0.175 | −31.82% | 1.03 | 0.8985 |
| Vascularity | −0.25 | −14.45% | 0.866 | 0.2699 |
| Obstruction | −0.4 | −20.25% | 0.995 | 0.1114 |
| Turbidity | −0.65 | −22.03% | 0.630 | 0.0010 |
| Zone A | −0.425 | −11.97% | 0.832 | 0.0248 |

*One patient was excluded from vision since he had prosthesis in his right eye

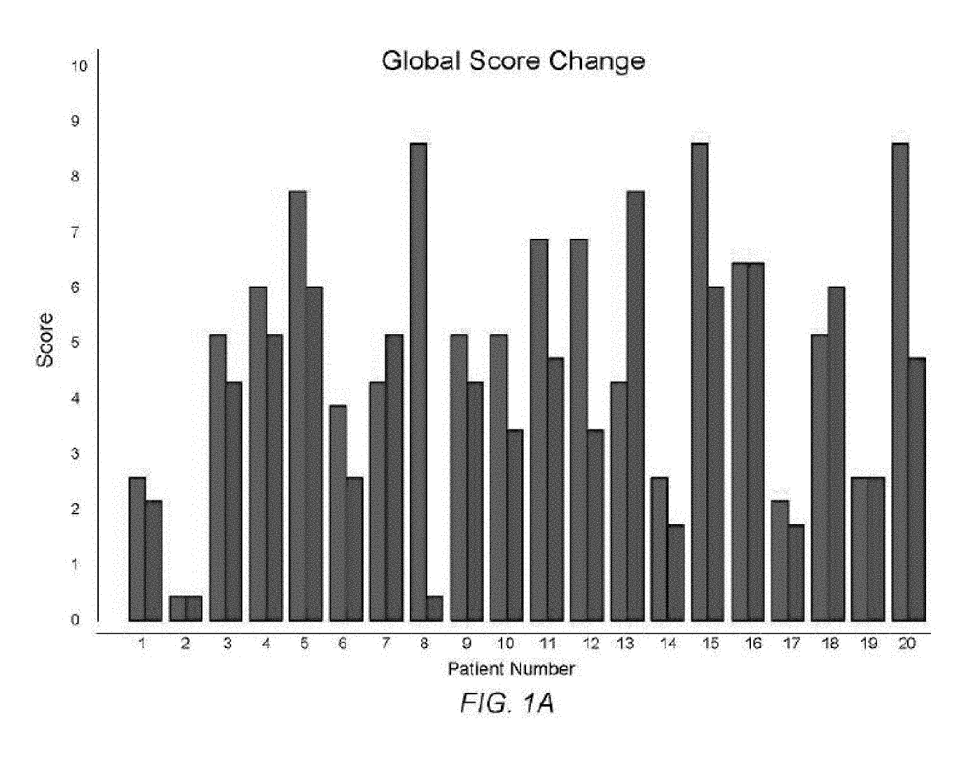

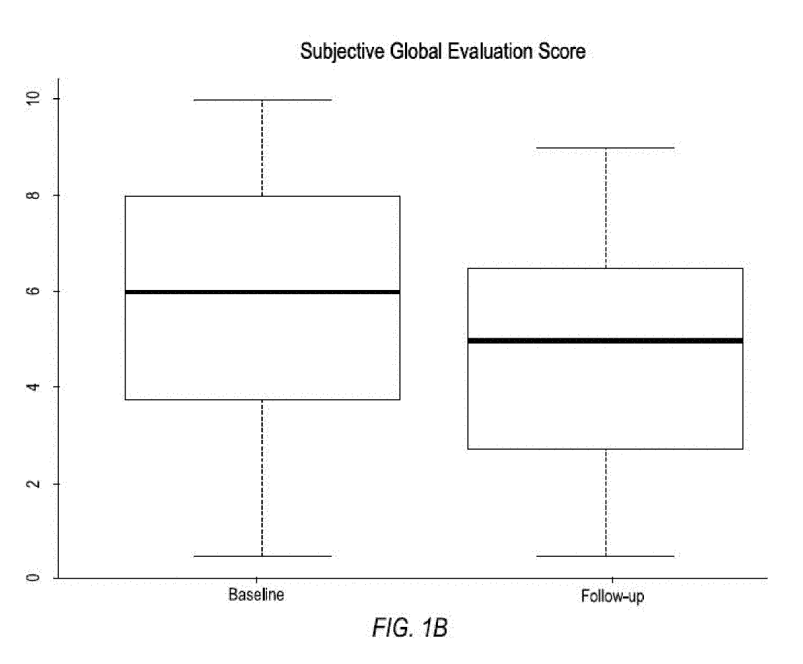

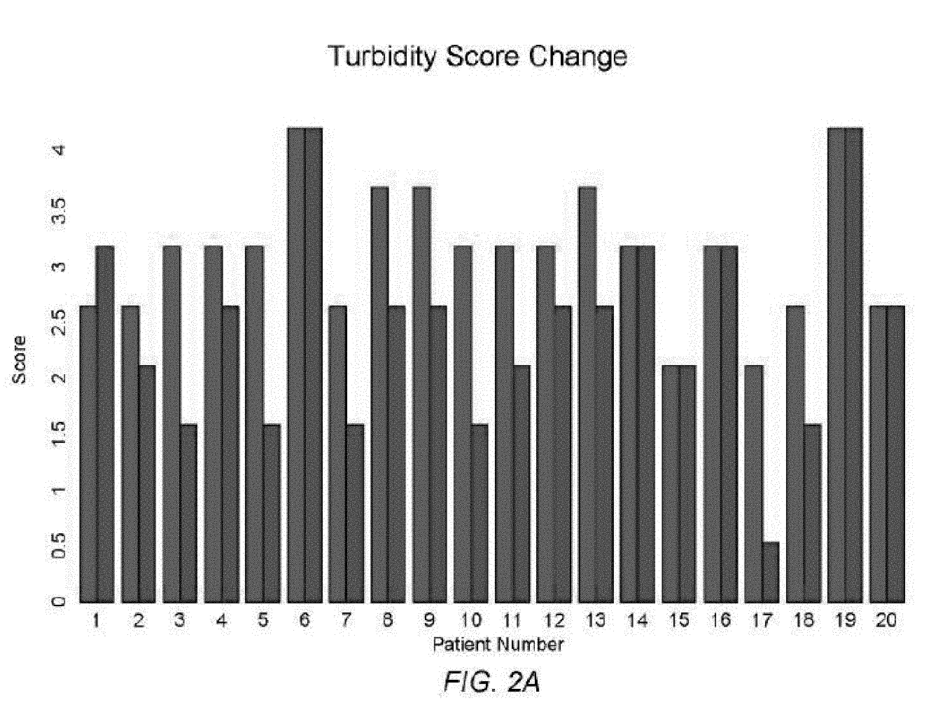

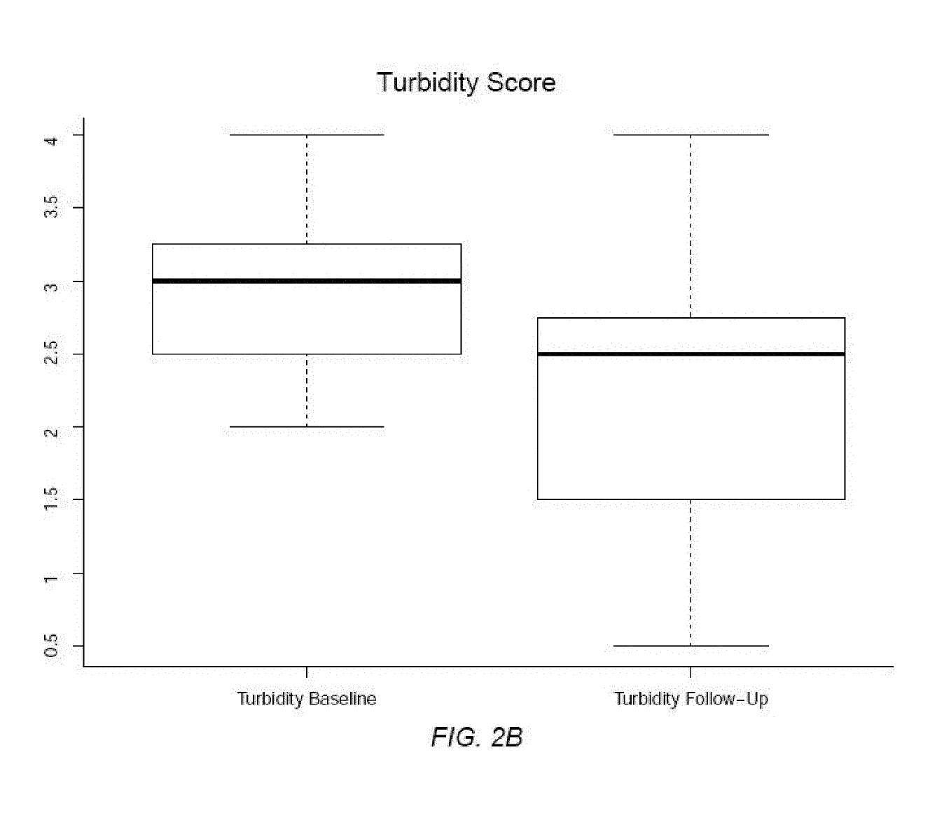

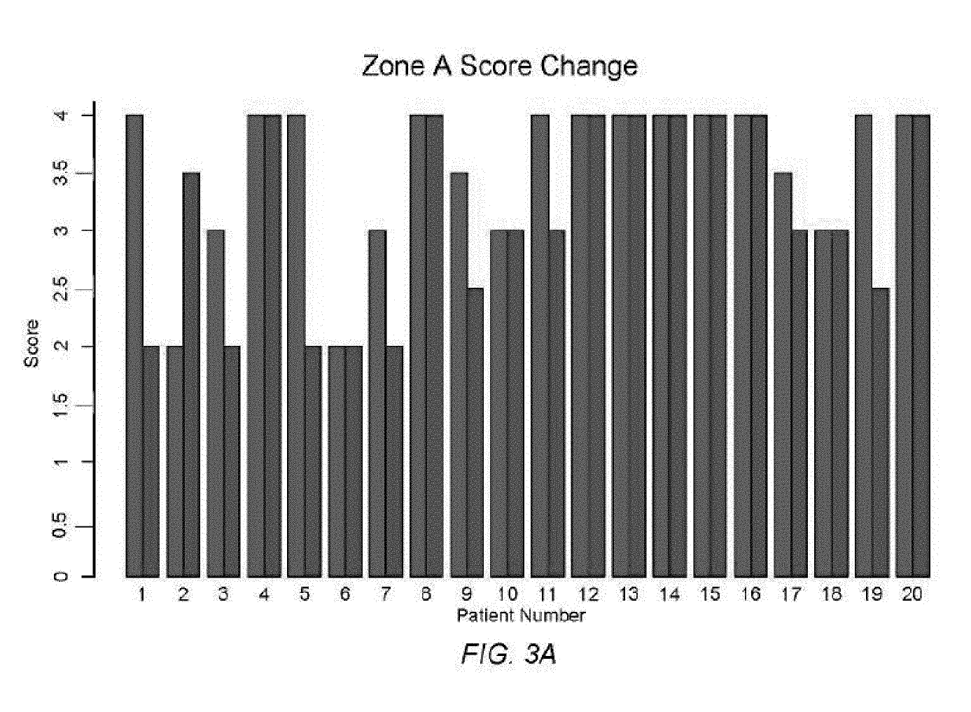

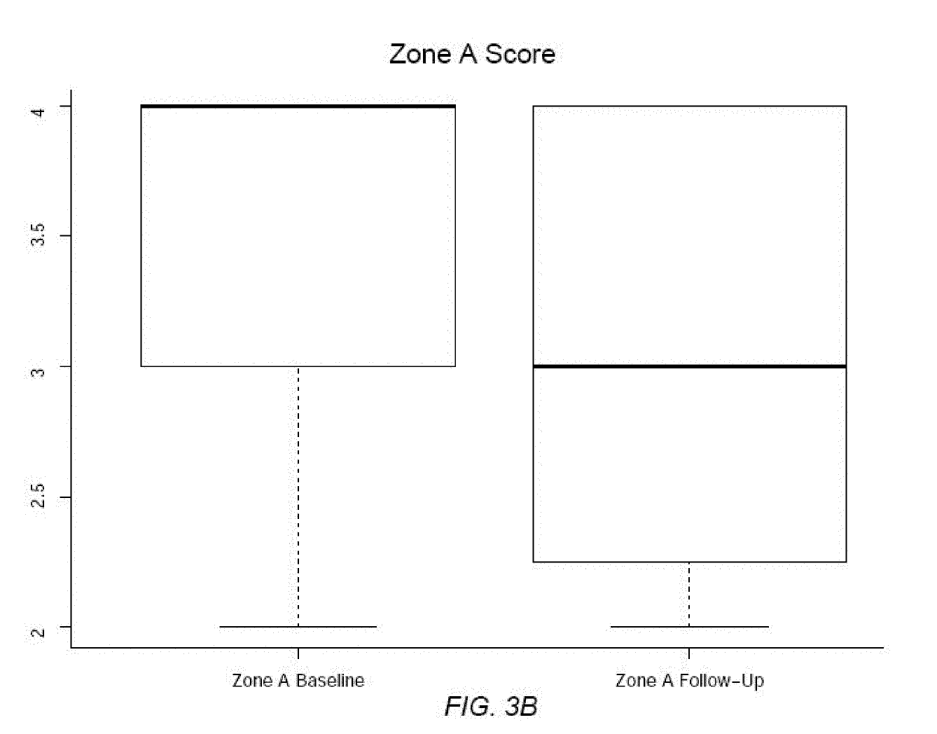

The change in statistically significant measurements (global evaluation, turbidity, and zone A) is plotted in FIGS. 1A-3B. As noted, the majority of the parameters measured showed trends of improvement.

Discussion: [000186] This study is the first to evaluate the potential of topical spironolactone to treat MGD. This study is expected to be published in 2016 as “Topical Spironolactone in the Treatment of Meibomian Gland Dysfunction,” by Brian S. Wong, Mikhail de Jesus, Richard W. Yee. Based on the results shown in Table III, application of a topical spironolactone ophthalmic suspension has a statistically significant effect (p<0.05) on the subjective global assessment score, keratitis and conjunctival score in the nasal region of the right eye, turbidity grade, and the zone A grade. The change in global evaluation score (p=0.0113) suggests that patients' symptoms associated with dry eye syndrome (redness, swelling, irritation) decreased, as well as better oil production, lower levels of inflammation, and less gland obstruction. The most statistically significant change is in the turbidity grade (p=0.0010). This change suggests that spironolactone may have a role in changing the quality of the expressed meibum through various mechanisms based on its diverse pharmacological properties. Moreover, change in zone A grade (p=0.0248) may reflect the anti-inflammatory properties of spironolactone.

Spironolactone was first used as a potassium-sparing diuretic due to its antagonistic activity at aldosterone receptors. Many other properties of spironolactone have been discovered including its dual activity at testosterone receptors (see Térouanne, et al., “A stable prostatic bioluminescent cell line to investigate androgen and antiandrogen effects,” Molecular and Cellular Endocrinology, 160 (1-2): 39-49, (2001)). Due to this property, spironolactone has been used off-label for the treatment of hormonal acne in women and to suppress unwanted effects of androgens in individuals undergoing gender reassignment. Since testosterone has a known role in the development and function of meibomian glands, it is reasonable to assume that spironolactone may have additional off-label uses and may benefit patients with MGD.

The presence of MGD in patients with low testosterone levels was identified in 2002 and subsequent studies have confirmed the influence of androgens on gene regulation in meibomian glands (see Sullivan, et al., “Androgen deficiency, Meibomian gland dysfunction, and Evaporative dry eye,” Ann N Y Acad Sci., 966:211-222, (2002)). A study in 2011, showed that levels of testosterone were elevated in patients with MGD and suggested that elevated levels of testosterone should be used as a diagnostic criteria for diagnosing MGD (see Sahin, et al., “Meibomian Gland Dysfunction: Endocrine Aspects,” ISRN Ophthalmology, vol. 2011, Article ID 465198, 6 pages, (2011)). After an improvement in the understanding of MGD, one explanation of these conflicting results is that Sullivan was studying hyposecretory states resulting in MGD, while Sahin was studying hypersecretory states resulting in MGD. This suggests that it is necessary to have a proper balance of testosterone levels for meibomian glands to secrete meibum with an optimal quality for maintaining tear film. Spironolactone's property as a weak partial agonist of testosterone may help maintain a balanced level of testosterone. This property of spironolactone may address the underlying cause of MGD pathogenesis in addition to addressing the symptoms that may be associated with its anti-inflammatory properties.

An advantage of using topical spironolactone, rather than oral spironolactone, is that a lower concentration is necessary to deliver an effective dose directly to the site of action. Topical use of spironolactone still has possible side effects. A small percentage of the patients reported a mild temporary burning sensation in the eye after administration of spironolactone. The role of the anti-aldosterone activity of spironolactone in the eye is unclear. The presence of a renin-angiotensin system has been identified as a potential target for lowering intraocular pressure in patients with glaucoma. Strain and Chaturvedi, “The renin-angiotensin-aldosterone system and the eye in diabetes,” J Renin Angiotensin Aldosterone Syst., 3:243-246, 2002.

### Example 4

### Administering Dapsone and Spironolactone to a Subject

In many patients, dapsone and spironolactone, when administered topically in conjunction with one another, results in an additive effect (i.e. 10% to 30% improvement) in subjective complaints of surface symptoms and objective findings of overall improved inflammation based on vascularity, zone A scores and bulbar and palpebral injection. Dapsone and spironolactone can be administered simultaneously, sequentially, in the same or different compositions. There may or may not be improvement of the keratitis seen with surface disease depending on the severity of the aqueous components and how much morbidity that has occurred with essential macro and micro components of the ocular surface anatomy. Dapsone, however, may be contraindicated in patients with a well-documented sulfa allergy.

An exemplary 15 mL ophthalmic solution composition comprising 3 mg spironolactone and 2.5 mg dapsone per mL can be prepared as follows. Dissolve about 0.31 grams spironolactone and about 0.26 grams dapsone in an alcohol. Ethyl alcohol can be used from 70-95% ethyl alcohol, such as from 80-90%, or 85-95%. Return solution to syringe for filtering into a sterile glass mortar in a clean room. Approximately 8 mL of ethyl alcohol is needed for each 0.3 grams of spironolactone powder. Prepare an extra 1 mL syringe comprising an alcohol, such as from 70-95% ethyl alcohol. Using a filter, such as a 0.1, 0.2, 0.3, 0.4, 0.5 micron Teflon filter, filter the solution into a sterile glass mortar and rinse the filter with the extra alcohol (e.g., from 1-5 mL extra alcohol) to remove any spironolactone left in the filter. Place 1 mL of alcohol into the filtered solution and allow the alcohol to evaporate (typically several hours, to overnight) to leave a sterile powder in the glass mortar. Draw up 30 mL spironolactone vehicle (such as hypromellose 0.01% to 5%, such as from 0.05% to 0.8%, or from 0.1% to 0.5%, or from 0.2% to 1%, or from 0.3% to 2%, or from 0.4% to 3%, or from 0.5% to 4%, or 5%, with or without NaCl (i.e., preservative free, PF)) (see also Example 5 below for exemplary vehicle) using a 30 mL syringe from a sterile Pyrex bottle. In the same glass mortar, add drop by drop from 10 mL, to 20 mL, to 30 mL, to 40 mL, to 50 mL of spironolactone vehicle or spironolactone-PF vehicle up to the desired concentration, stirring/grinding vigorously. For a composition comprising about 2.5 mg dapsone and 3 mg spironolactone per mL, the amount of vehicle added is around 90-100 mL, such as about 91, 92, 93, 94, 95, 96, 97, 98, 99, or 100 mL. Continue to stir/grind well to make a paste, then a suspension. In the same sterile Pyrex bottle, add the syringe of the suspension and let spin, for example for up to 1, 2, 3, 4, 5, 6, 7, 8, 9, or 10 hours. Final pH of the composition is in the range of a pH of about 4-8, such as a pH of 4, 4.5, 5, 5.5, 6, 6.5, 7, 7.5, or 8. Transfer contents of pyrex bottle into an appropriate sized droptainer, for example, a 15 mL droptainer.

### Example 5

### Exemplary Aldosterone Antagonist Vehicle

An ophthalmic vehicle for administering aldosterone antagonist, such as spironolactone, can be prepared by the following method. Heat about 80-100 mL of water for irrigation in a beaker until steaming, about 70-120 degrees Celsius, such as about 70-110 degrees Celsius, or from about 75-115 degrees Celsius, or from about 80-105 degrees Celsius. Add about 0.04-0.06 grams (such as 0.0.045, 0.05, 0.055, 0.06 grams) edetate disodium dihydrate; about 0.4-0.6 grams (such as 0.45, 0.48, 0.50, 0.52, 0.55, 0.57, 0.59, 0.6 grams) dibasic sodium phosphate; about 0.05-0.2 grams (such as 0.06, 0.08, 0.10, 0.12, 0.14, 0.16, 0.18, or 0.2 grams) monobasic sodium phosphate; and about 0.5-3 grams (such as 0.8, 1, 1.2, 1.4, 1.7, 2, 2.2, 2.5, 2.8, or 3 grams) potassium chloride and stir until dissolved. Add about 0.05-0.8 grams (such as 0.08, 0.1, 0.15, 0.2, 0.25, 0.3, 0.35, 0.4, 0.5, 0.6, 0.7, or 0.8 grams) methocel to the heated solution and stir until clear. Bring to proper volume with water for irrigation. Add citric acid to adjust to desired acidic pH of about 4, 4.5, 5, 5.5, 6, 6.5, such as by using from 0.1-0.5 grams (such as 0.15, 0.2, 0.25, 0.3, 0.35, 0.4, 0.45, or 0.5 grams) citric acid. Transfer solution to an amber pyrex bottle. Close cap. Autoclave the solution until the solution reaches a temperature ranging from about 110 degrees Celsius to 130 degrees Celsius, such as up to 110, 111, 112, 113, 114, 115, 116, 117, 118, 119, 120, 121, 122, 123, 124 degrees Celsius and so on. Pressure can be in the range of 5-30 PSI, such as from 5-25 PSI, or from 10-20 PSI, or from 15-25 PSI, and so on. The residence time in the autoclave can range from about 20 minutes to 2 hours, such as from 30, 40, 45, 50, 55, 60, 75, 80, 90, 100, 110, or 120 minutes. The suspension can be stirred while autoclaving and/or cooling. Transfer the solution to a droptainer, such as a 10 mL, 15 mL, 20 mL, or 30 mL droptainer as appropriate. Final pH of the composition is in the range of a pH of about 4-8, such as a pH of 4, 4.5, 5, 5.5, 6, 6.5, 7, 7.5, or 8.

### Example 6

### Lipid Production in Corneal Epithelial Cells—Dilutions of 3 mq/ml Spironolactone Formulation

Corneal epithelial cells were cultured in vitro and treated with 0.03 mg/ml and other dilutions of a 3 mg/ml formulation of Spironolactone (100× (0.03 mg/ml), 200× (0.015 mg/ml), 500× (0.006 mg/ml), and 1000× (0.003 mg/mI)). The corneal epithelial cells were cultured in vitro to about 10-20% confluence. The cell medium with added spironolactone was changed every 2-3 days. On the ninth day, the cells were stained for lipids with Oil Red O.

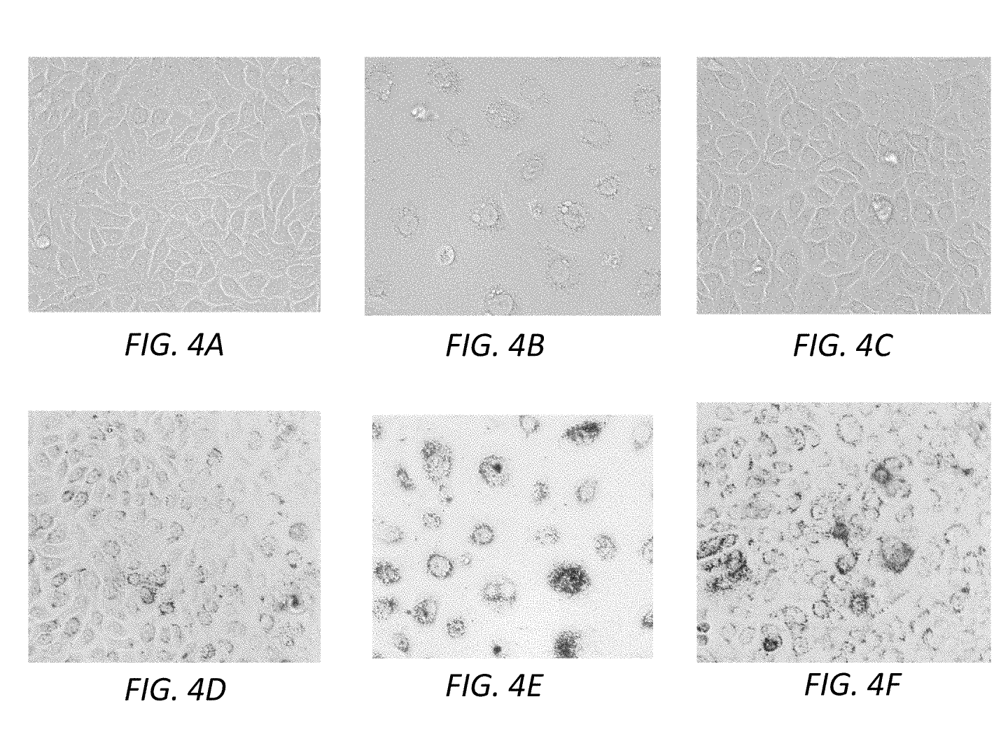

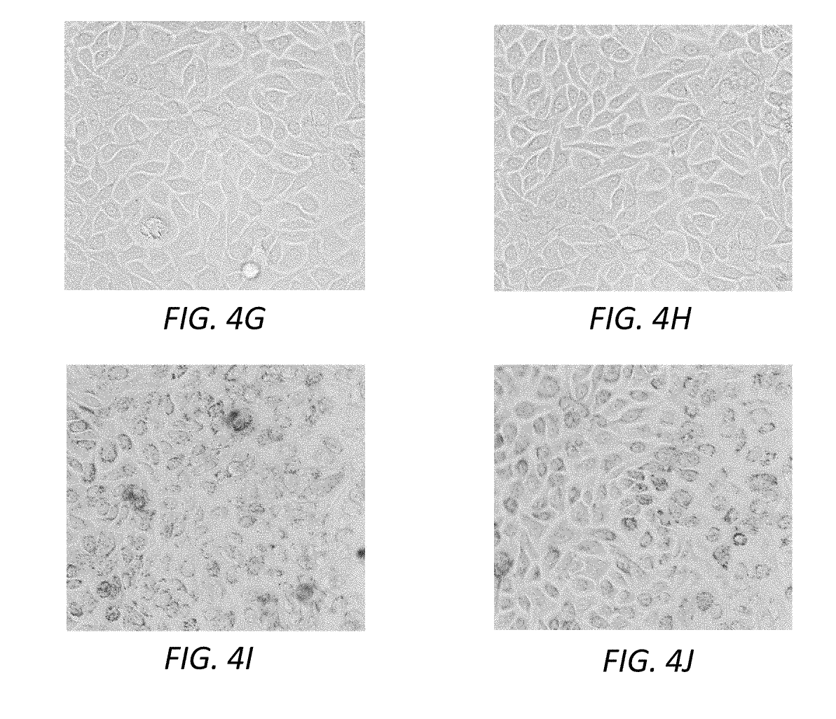

FIGS. 4A-4C show microscopic images of control, 0.03 mg/ml, and 0.015 mg/ml treated cells, while FIGS. 4D-F show microscopic images of control, 0.03 mg/ml, and 0.015 mg/ml treated cells which were stained for lipids with Oil Red O. FIGS. 4G and 4H show microscopic images of 0.006 mg/ml and 0.003 mg/ml treated cells, while FIGS. 4I and 4J show microscopic images of 0.006 mg/ml and 0.003 mg/ml treated cells which were stained for lipids with Oil Red O. As shown in FIGS. 4E, 4F, and 4I, the microscopic images of treated cells (0.03, 0.015, and 0.006 mg/ml groups) show enhanced lipid production in comparison to control (FIG. 4D).

### Example 7

### Lipid Production in Corneal Epithelial Cells—Dilutions of 0.025 mq/ml Spironolactone Formulation

Corneal epithelial cells were cultured in vitro to about 30% confluence and treated with various dilutions of a 0.025 mg/mL spironolactone eye drop formulation (i.e. 50×, 100×, 500×, 1000×, and 5000×, corresponding to 0.0005 mg/ml, 0.00025 mg/ml, 0.00005 mg/ml, 0.000025 mg/ml, and 0.000005 mg/ml). The cell medium with spironolactone was changed every two days. On the seventh day, the cells were stained for lipids with Oil Red O.

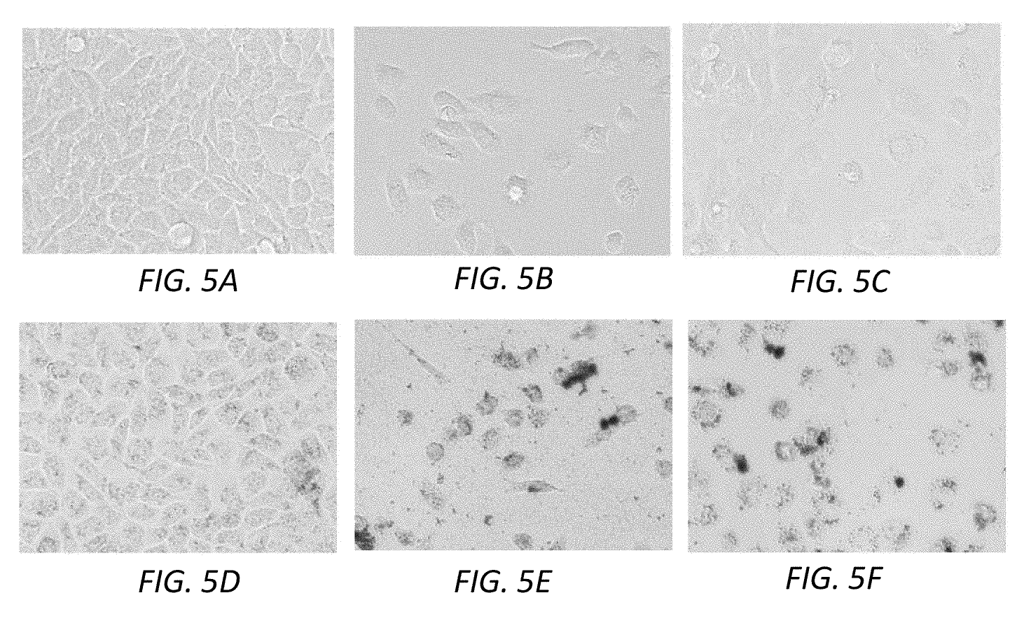

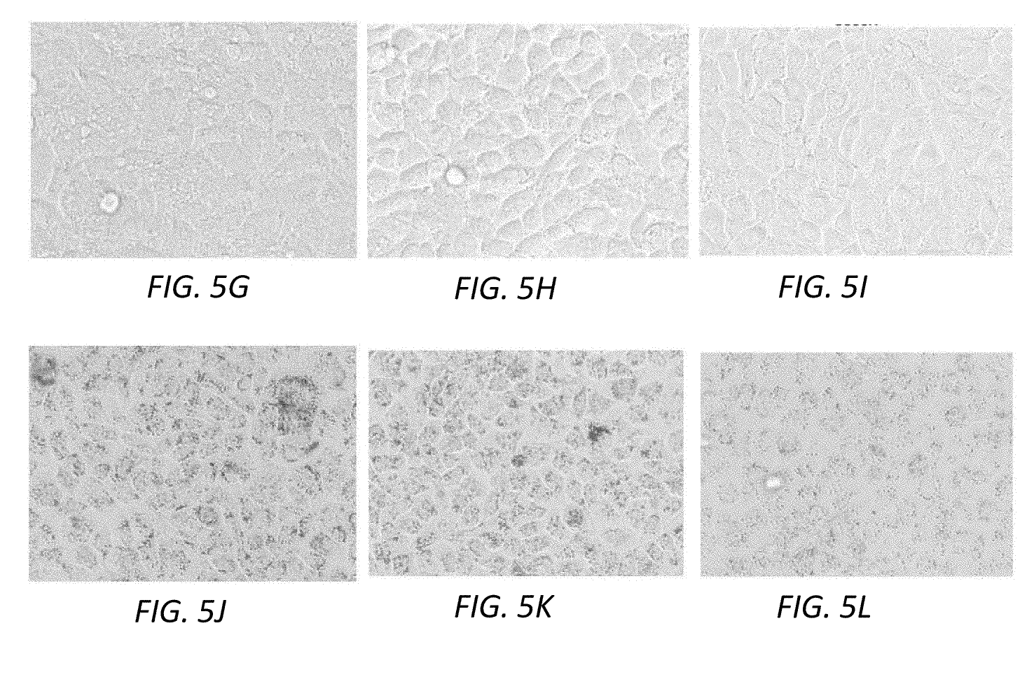

The results are shown in FIGS. 5A-5L. The top rows (FIGS. 5A-C, 5G-5I) represent unstained cells showing cell morphology, and the bottom rows (FIGS. 5D-F, 5J-5L) represent cells stained for lipids. FIGS. 5A and 5D represent the control group (no spironolactone), FIGS. 5B and 5E represent treatment with a 50× dilution (0.0005 mg/ml), FIGS. 5C and 5F represent a 100× dilution (0.00025 mg/ml), FIGS. 5G and 5J represent a 500× dilution (0.00005 mg/ml), FIGS. 5H and 5K represent a 1000× dilution (0.000025 mg/ml), and FIGS. 5I and 5L represent a 5000× dilution (0.000005 mg/ml).

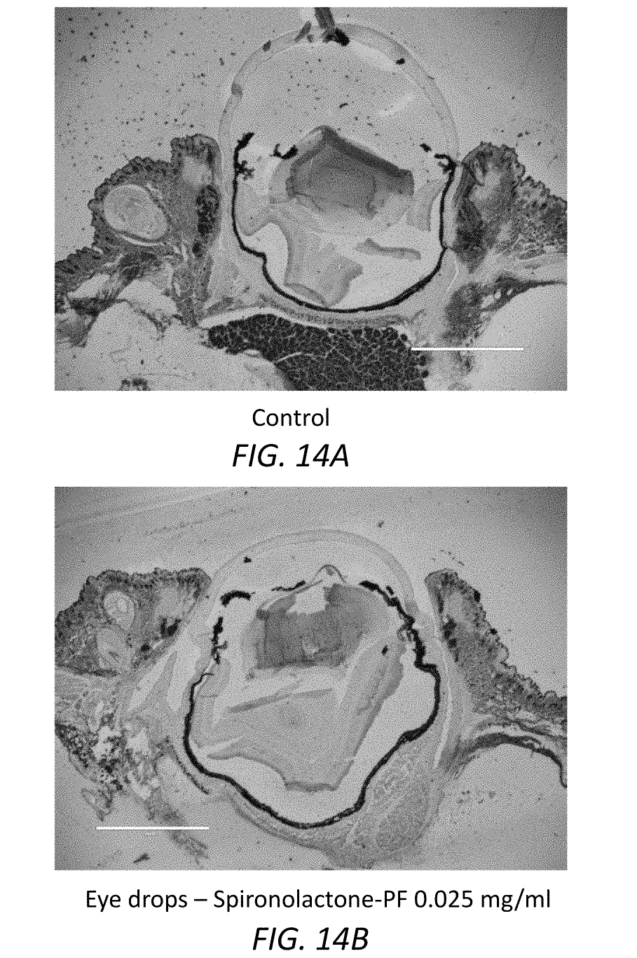

Toxicity to cells was also taken into account. Toxicity in the context of this disclosure refers to any amount of cell death upon exposure to the composition. Little to no toxicity or relatively no toxicity refers to some amount of cell death to no cell death upon exposure to the composition. Alternatively or additionally, the morphology should look phenotypically normal for the cells. Toxicity can be measured quantitatively or qualitatively, in vivo or in vitro. Standard assays, such as an LDH (lactate dehydrogenase) assay, can be used to quantify toxicity of the compositions and to determine a TC₅₀ concentration, i.e., a concentration that results in cell death for 50% of the cells upon exposure of the cells to that concentration. Depending on the application and the particular physiological circumstances of a patient, concentrations that exhibit toxicity in vitro can still be used in vivo, such as with a patient who produces a plethora of tears such that the concentration of the composition upon administration to the patient would be expected to be diluted further. For example, although some in vitro toxicity was observed at 0.025 mg/ml, in vivo tests showed that eye drops with a concentration of spironolactone in the amount of 0.025 mg/cc administered twice a day for more than 25 days does not show any difference with the control eye (i.e., no gross changes compared with the control), which suggests no deleterious effect in the normal in vivo animal model. See FIGS. 14A and 14B. As such, a concentration of 0.025 mg/cc or lower appears to be safe. At a 1000× dilution (0.000025 mg/ml), no in vitro toxicity was observed but the lipid production was still present. At a 5000× dilution (0.000005 mg/ml), the results are similar to the control group in that no lipid is being formed. Compositions with lower amounts of active agent (e.g., 0.0000025 mg/cc) may be useful for maintaining a patient's condition, without subjecting the patient to long-term toxicity, for example, once the patient has been stabilized by treatment for some amount of time with a higher concentration.

### Example 8

### Clinical Trial

Summary:

Purpose: Conventional treatments for dry eye syndrome have focused on addressing tear levels and inflammation, but have failed to demonstrate efficacy in all patients. New therapies have increasingly addressed meibomian gland dysfunction (MGD). This study tested the hypothesis that topical spironolactone can aid in the treatment of MGD by regulating and improving sebaceous gland meibum secretions through a variety of mechanisms in both patients with and without aqueous tear deficiency.

Methods: We performed a retrospective, observational clinical study of 102 patients with moderate to severe MGD from November 2014 to May 2016. These patients were split into two cohorts: the first cohort included 75 patients with Schirmer's tear test (STT) scores greater than 5 and at least 1 follow-up after 1 month and the second cohort included 27 patients with STT of 5 or less and at least 1 follow-up after 1 month. Both cohorts were monitored for changes in subjective global dry eye assessment, keratitis and conjunctival staining, anterior blepharitis grade (AB), gland obstruction grade (O), meibum turbidity grade (T), vascularization of the anterior lid margin (V), zone A posterior lid margin grade, best corrected vision, and STT score. These parameters were compared in a pre-post study.

Results: At the first follow-up, the 75 patients in the first cohort had statistically significant improvements in self-reported global assessment scores (p<0.0001), V scores (p=0.0009), O scores (p=0.0011), T scores (p<0.0001), and Zone A scores (p<0.0001). At the first follow-up, the 27 patients in the 2nd cohort had statistically significant improvements in self-reported global assessment scores (p=0.030), V scores (p=0.0056), T scores (0=0.0005), Zone A scores (p=0.0199), and STT (p=0.0324 OD, p=0.0096 OS).

Conclusions: In both cohorts at the 1st follow-up, patients reported improved dry eye symptoms after using spironolactone. The quality of expressed meibum secretions showed clinical improvement of clarity and viscosity posttreatment. Inflammation decreased at the avascular region ˜0.5 mm posterior to the posterior lid margin. Patients with low STT prior to treatment showed increased tear quantity. Ultimately, this retrospective study demonstrates the potential for spironolactone to significantly regulate meibum quality and address inflammation in treating moderate to severe MGD.

Description:

Background

Conventional treatments for dry eye syndrome have focused on addressing tear levels and inflammation, but have failed to demonstrate efficacy in all patients. New therapies have increasingly addressed meibomian gland dysfunction (MGD). Topical spironolactone is a drug with low toxicity and the potential to regulate and improve sebaceous gland meibum secretions through a variety of mechanisms.

Purpose

The objective of this study was to investigate the effectiveness of topical spironolactone in treating MGD, a major component of dry eye syndrome.

Design

Retrospective cohort study.

Methods Setting: Clinical practice.

Patient Study Population: The use of Spironolactone (3 mg/ml) for treating Meibomian Gland Dysfunction (MGD) was evaluated in a patient population (n=102) with severe MGD. 102 patients from November 2014 to May 2016 with moderate to severe meibomian gland disease were included in this study. Four different cohorts were included in this study. The first cohort included 75 patients with Schirmer's test scores greater than 5 and with at least 1 follow-up, and the second cohort included 27 patients with Schirmer's test scores of 5 or less prior to the start of topical Spironolactone and with at least 1 follow-up. The third cohort included the 28 patients from the first cohort who had more than one follow-up, and the fourth cohort included the 10 patients from the second cohort who had more than one follow-up. In all four groups, we excluded patients who were taking glaucoma medications, steroid eye drops and other lipid-altering eye drops prior to starting spironolactone. In addition, patients who had autoimmune diseases causing dryness, such as Sjogren's syndrome and Sicca syndrome were excluded. We also excluded patients who did not use the topical Spironolactone drops at least twice a day in both eyes at the start of the treatment and until the first follow-up appointment. Patients who had turbidity scores of less than 2 and Meibo scores of greater than 3 prior to the start of topical Spironolactone were also excluded. We analyzed several parameters in describing MGD. These included subjective global dry eye assessment, keratitis and conjunctival staining, anterior blepharitis grade, gland obstruction grade, meibum turbidity grade, meibum viscosity grade, zone A posterior lid margin grade, best corrected vision, tear osmolarity, and Schirmer's score. We compared these parameters in a pre-post study. These parameters were tracked for up to four follow-ups. The first follow-up times ranged from 1 to 7 weeks, with an average of approximately 3 weeks. All subsequent follow-up times were 3 months after the previous follow-up. [000217] Main outcome measures: Parameters in the prevalence of meibomian gland dysfunction (subjective global assessment, lissamine green keratitis and conjunctival staining, anterior blepharitis grade, obstruction grade, vascularity grade, turbidity grade, zone A grade, vision, tear osmolarity, and Schirmer's tear score) in patients with moderate to severe MGD.

Results

After the first follow up, the 75 patients with Schirmer's test scores greater than 5 had improved self-reported global assessment scores (p=0.000), vascularity scores (p=0.0009), obstruction scores (p=0.0011), turbidity scores (p=0.000), and Zone A scores (p=0.000) after treatment with topical ophthalmic suspensions of spironolactone. After the final follow up for the 28 patients with more than 1 follow up, in comparison to the first follow up, the self-reported global assessment scores (p=0.0088) and vascularity scores (p=0.0409) continued to show significant improvement while the other studied variables continued to improve, but not at a statistically significant level.

After the first follow up, the 27 patients with Schirmer's test scores of 5 or less had improved self-reported global assessment scores (p=0.0297), vascularity scores (p=0.0056), turbidity scores (p=0.0005), Zone A scores (p=0.0199), and Schirmer's test scores (p=0.0324 OD, p=0.0096 OS) after treatment with topical ophthalmic suspensions of spironolactone. After the final follow up for the 10 patients with more than 1 follow up, in comparison to the first follow up, the Schirmer's OD score continued to improve (p=0.0479), while many of the other studied variables did not show the same improvement over time.

Conclusions

MGD patients reported improved dry eye symptoms after using compounded topical spironolactone ophthalmic suspensions for at least one month. The quality of expressed meibum secretions of MGD patients clinically showed improved clarity and viscosity post-treatment. Inflammation decreased at the avascular region ˜0.5 mm posterior to the posterior lid margin post-treatment. For patients who had low Schirmer's scores prior to beginning the treatment, in addition to the aforementioned improvements, there was also an increase in tear quantity observed as soon as one month post-treatment. MGD patients with multiple follow ups and Schirmer's scores of greater than 5 showed continued improvement in the initially improved parameters. However, this trend was not as prevalent in the MGD patients with low Schirmer's scores who had multiple follow ups. These discrepancies could be due to the low sample size of these 2 groups, and suggests further studies to test for the long-term effectiveness of Spironolactone on Meibomian Gland Dysfunction. Ultimately, this study demonstrates the potential for spironolactone to regulate meibum quality and address inflammation in treating MGD.

Meibomian Gland Dysfunction (MGD) is a major contributor to dry eye syndrome and characterized by diminished function of meibomian glands. These glands normally release an oily secretion to maintain tear film stability. The meibomian glands of affected individuals are often characterized by terminal duct obstruction, glandular secretion changes, and posterior lid margin inflammation (see Nelson J D, Shimazaki J, Benitez-del-Castillo J M, Craig J P, McCulley J P, Den S, et al. The international workshop on meibomian gland dysfunction: report of the definition and classification subcommittee. Invest Ophthalmol Vis Sci. 2011;52(4):1930-7. doi: 10.1167/iovs.10-6997b). MGD has been diagnosed based on presence of glandular dropout, reduced secretion upon gland expression, meibum secretion quality, inflammation, and meibography (see Foulks G N, Bron A J. Meibomian gland dysfunction: a clinical scheme for description, diagnosis, classification, and grading. Ocul Surf. 2003; 1:107-126) Arita recommends physicians to suspect obstructive MGD with two or more abnormal scores in ocular symptoms, lid margin abnormalities, and meibo-score (see Arita R, Itoh K, Maeda S, et al. Proposed diagnostic criteria for obstructive meibomian gland dysfunction. Ophthalmology. 2009; 116:2058-2063).

Current treatments for MGD include warm compresses, lid hygiene, intraductal meibomian gland probing, lipid-emulsion eye drops, thermal pulsation, n-acetyl-cysteine, azithromycin, omega 3-fatty acid supplementation, cyclosporine A eye drops, and intense pulse-light therapy (IPL) (see Craig, J P, Chen Y, Turnbull P R K. Prospective Trial of Intense Pulsed Light for the Treatment of Meibomian Gland Dysfunction. Investigative Ophthalmology & Visual Sciences. 2015; 56(3):1965-70; Qiao J, Yan X. Emerging treatment options for meibomian gland dysfunction. Clin Ophthalmol. 2013; 7:1797-803). While current treatments target symptoms, they are sometimes poorly tolerated and often offer temporary relief. Spironolactone is a drug that has been used as a potassium-sparing diuretic to treat heart failure for over 35 years (see Rathnayake D and Sinclair R, Use of spironolactone in dermatology. Skinmed. 2010 November-December; 8(6):328-32; quiz 333; “Rathnayake D and Sinclair R”). In recent years, its anti-androgenic effects have been used in dermatologic settings to treat hirsutism, female pattern hair loss, and hormonal acne in women (see Rathnayake D and Sinclair R; and Salavastru C M, Fritz K, Tiplica G S [Spironolactone in dermatological treatment. On and off label indications.] Hautarzt. 2013 October; 64(10):762-7. doi: 10.1007/s00105-013-2597-y). Spironolactone is a synthetic 17-lactone steroid with anti-androgenic and anti-inflammatory properties in addition to its anti-hypertensive effect (see JérômeFagart, Alexander Hillisch, Jessica Huyet, Lars Bärfacker, Michel Fay, Ulrich Pleiss, Elisabeth Pook, Stefan Schäfer, Marie-Edith Rafestin-Oblin, and Peter Kolkhof A New Mode of Mineralocorticoid Receptor Antagonism by a Potent and Selective Nonsteroidal Molecule. J. Biol. Chem. 2010 285: 29932-29940. First Published on Jul. 22, 2010, doi:10.1074/jbc.M110.131342). Its anti-androgenic effect is due to a number of mechanisms including regulating androgen receptors. Unlike anti-androgenic drugs that are testosterone receptor antagonists, spironolactone is unique in that it is a weak partial agonist at androgen receptors and may have an agonistic or antagonistic effect at androgen receptors, depending on the concentration of testosterone. In individuals with normal testosterone levels, spironolactone inhibits key enzymes (17α-hydroxylase) in the androgen biosynthetic pathway, activates the progesterone receptor, and inhibits 5α-reductase, a key enzyme in the synthesis of dihydrotestosterone (DHT), a potent androgen (see Corvol P, Michaud A, Menard J, et al. Antiandrogenic Effect of Spironolactones: Mechanism of Action. Endocrinology 1975 97:1, 52-58). Due to spironolactone's dual anti- and pro-testosterone properties, anti-aldosterone, and anti-inflammatory properties, it is reasonable to believe that spironolactone may improve the quality of meibomian gland secretions and address the inflammation seen in patients with MGD.

Spironolactone has been associated with adverse effects such as increased urinary frequency, hyperkalemia, rashes, and menstrual irregularities in women (see. Greenblatt D J, Koch-Weser J. Adverse Reactions to Spironolactone: A Report From the Boston Collaborative Drug Surveillance Program. JAMA. 1973; 225(1):40-43. doi:10.1001/jama.1973.03220280028007) Adverse effects tend to be dose-related, however, the long-term use of spironolactone appears to be safe (see Shaw J C, White L E. Long-term safety of spironolactone in acne: results of a 8-year follow up study. J Cutan Med Surg. 2002 November-December; 6(6):541-5. Epub 2002 Sep. 12). The use of spironolactone in an ocular vehicle has never been previously reported in the literature, but may reduce the risk for adverse effects while improving the quality of meibomian gland secretions in MGD by potentially decreasing systemic levels when compared to oral therapy. This is due in large part to a significantly lower dose required when instilling the drug at the site of action. To the best of our knowledge, the effect of topical spironolactone on MGD has never been studied or previously reported.

The primary objective of this study was to evaluate the efficacy of topical spironolactone in the treatment of ocular surface disease based on subjective global assessment scores, keratitis (KS) and conjunctival scores (CS), anterior blepharitis grade (AB), lid margin vascularity grade (V), obstruction grade(O), turbidity grade (T), zone A grade, and vision. In addition, the second objective of this study was to evaluate whether the efficacy of topical spironolactone differs in patients with aqueous tear deficiency, in comparison to patients who make adequate tears as seen in the Schirmer's tear scores (STS). The sustained effectiveness of Spironolactone over time was also a major question for this study.

Methods

This study is a retrospective chart review of 102 patients separated into two groups. The first group included 75 patients with Schirmer's test scores of greater than 5, and the second group included 27 patients with Schirmer's test scores of 5 or less. Two sets of comparisons were performed for the variables being studied. For the first comparison set, the measurements at the baseline visit were compared with the measurements at the first follow up visit. For the second comparison set, the measurements taken at the first follow up visit were compared to the measurements taken at the patients' final follow up visit, if the patient had more than one follow up. 28 patients with Schirmer's scores of greater than 5 had more than 1 follow up, while 10 patients with Schirmer's scores of 5 or less had more than 1 follow up. Corrected visual acuity was measured prior to starting spironolactone and during the follow-up visits. Corrected visual acuity was recorded in log MAR based on the corresponding line read correctly on the Snellen chart. Patients self-assessed dry-eye global scores based on the presence of symptoms during the current exam on a scale from 0 to 10 based on (0=no dry eye symptoms, 10=the worst dry eye symptoms ever felt). Keratitis and conjunctival scores were evaluated using lissamine green staining on nasal conjunctival, central corneal, and temporal conjunctival regions on a scale from 0 to 3 (0=no staining and 3=confluent staining). Schirmer's tests were performed without topical anesthetics to evaluate tear film production. Tear osmolarity tests were performed using 50 microliters of tears from each eye using the Tear Lab osmometer analyzer before and after treatment. Anterior blepharitis scores were evaluated using a slit lamp and graded on a scale from 0 to 4 (0=no dandruff and 4=dandruff across the entire lash line). Lid margin abnormalities such as vascularity and inflammation of the avascular region ˜0.5 mm posterior to the posterior lid margin (Zone A) were evaluated and graded on a scale from 0 to 4 (0=no vascularization and 4=vascularization of the entire margin) (see Arita, R., Zavala, M., & Yee, R. W. (2014, Jun. 4). MGD Diagnosis. Curr Opthalmol Rep, 49-57). Meibomian gland expression (obstruction) was evaluated by applying pressure to the lower lid and graded on a scale from 0 to 4. The quality of the expressed meibum (turbidity and viscosity) was also evaluated and graded from 0 to 4 (0=clear oil; 4=cloudy & toothpaste-like). The diagnosis of MGD was made based on the presence of a score of 3+ on symptoms or 2+ on lid margin abnormalities (see Arita R, Itoh K, Maeda S, et al. Proposed diagnostic criteria for obstructive meibomian gland dysfunction. Ophthalmology. 2009; 116:2058-2063). Patients with autoimmune diseases such as Sjorgen's syndrome as well as patients taking glaucoma medications or steroid eye-drops were excluded from the study. In addition, patients who had turbidity scores of less than 2 prior to the start of topical Spironolactone were also excluded. Patients included in the study started using spironolactone eye drops prior to using other topical eye medications or systemic medications to treat any dry eye conditions and continued using the Spironolactone eye drops at least twice a day in both eyes until the first follow-up appointment. Patients were prescribed spironolactone after previously taking omega-3 fatty acid and flax seed oil supplements and practicing blinking exercises with limited improvement. The average follow-up interval in this pre-post study was 1 month for the first follow up, and then every 3 months for each subsequent follow up. Statistical analysis was performed using STATA 13 by fitting scored data to a non-parametric model with the Wilcoxon signed-rank test and testing continuous data with a paired t-test.

b. Results

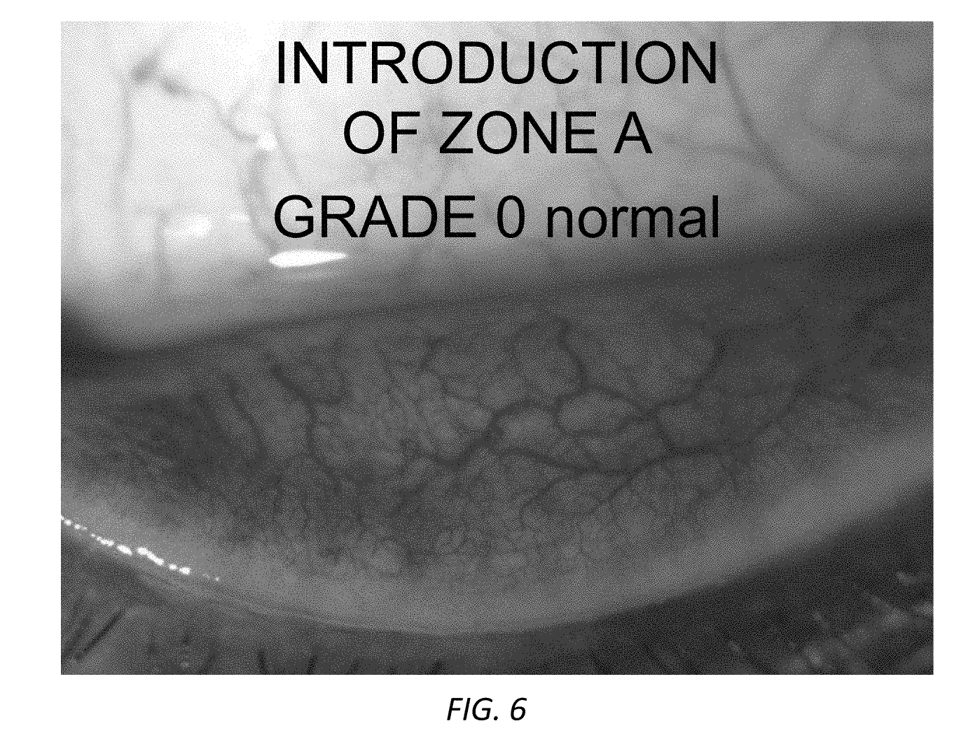

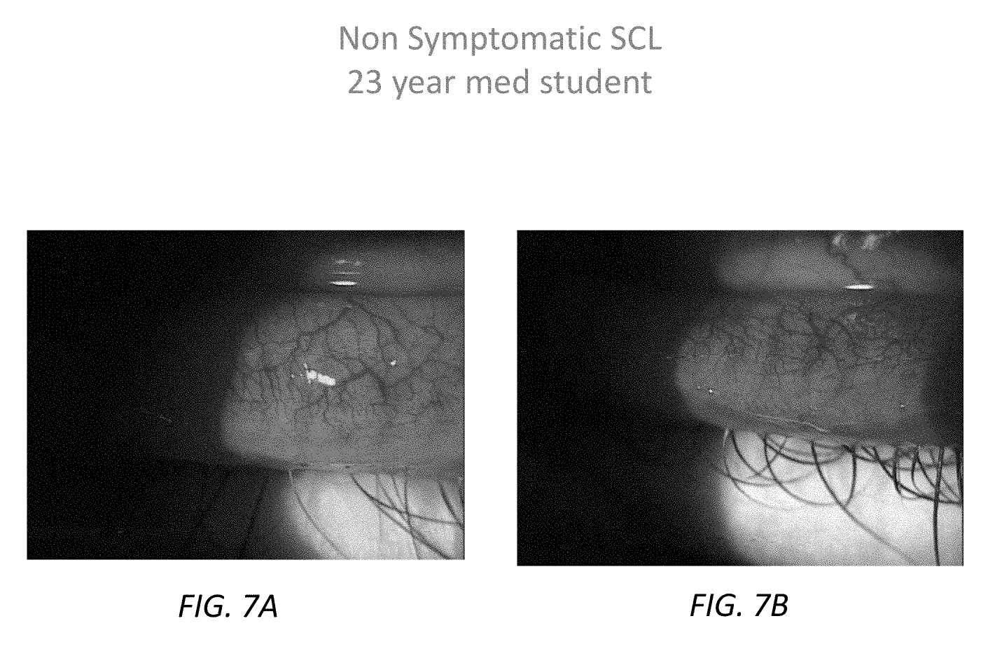

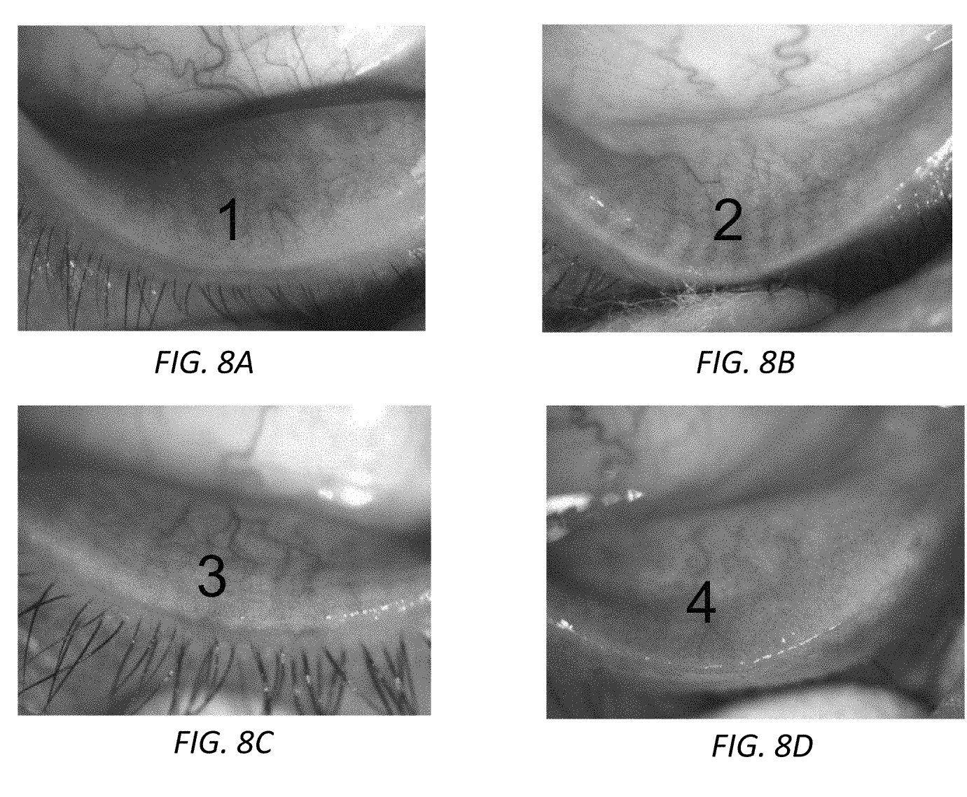

The image in FIG. 6 shows an example of a normal (grade 0) Zone A in a patient. The images in FIGS. 7A and 7B show additional examples of a normal (grade 0) Zone A in a 23 year old medical student. FIGS. 8A-8D shows Zone A with progressive levels of surface inflammation (graded 1 to 4, respectively).

Topical Spironolactone improved symptoms of ocular irritation of moderate to severe symptomatic MGD as early as 2 weeks (not 4-6 months like Restasis). Also improved were Lissamine Green staining, Schirmer's I test scores, Lid margin and Zone A levels of vascularity, the quality of the meibum and the relative obstruction of MG secretions.

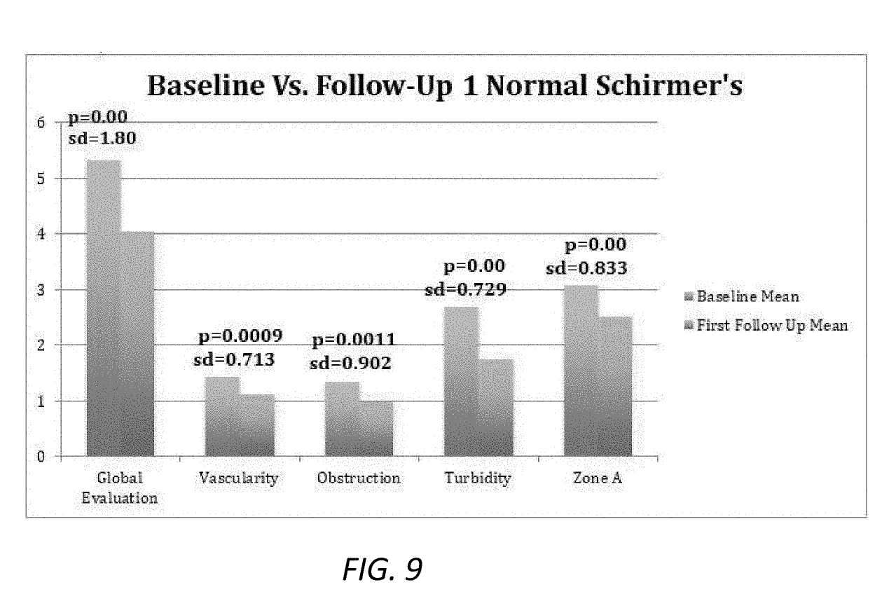

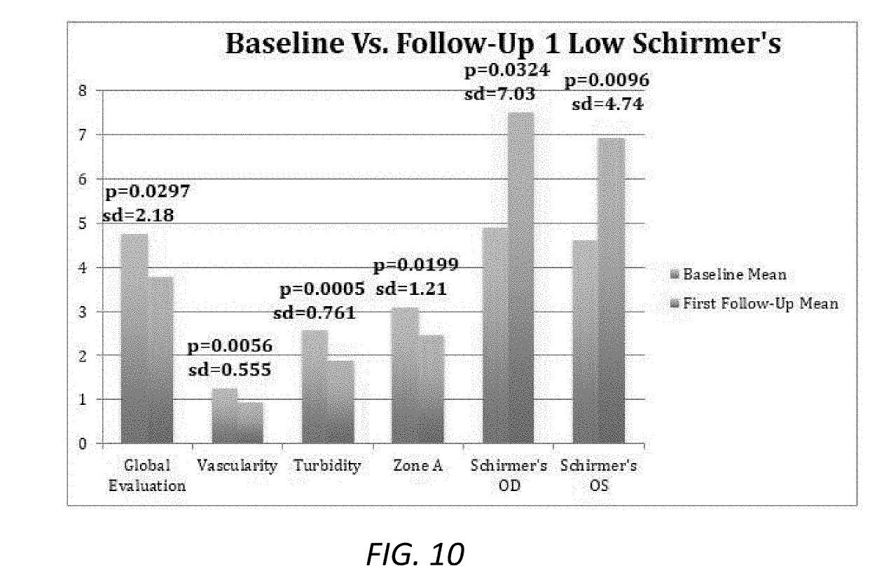

The pre-treatment measurements, post-treatment measurements, and change between pre- and post-treatment are summarized in Tables 1-12 below. FIG. 9 shows the statistically significant parameters in the comparison between the baseline visit and follow-up 1 for the 75 patients with normal Schirmer's scores. The percent reduction in Global Evaluation, Vascularity, Obstruction, Turbidity, and Zone A scores from baseline were 27.52%, 24.51%, 31.23%, 41.82%, and 19.86%. FIG. 10 shows the statistically significant parameters in the comparison between the baseline visit and follow-up 1 for the 27 patients with low Schirmer's scores. The percent reductions in Global Evaluation, Vascularity, Turbidity, and Zone A scores from baseline were 22.98%, 30.48%, 30.70%, 22.65%. Schirmer's I scores increased approximately 40% in these patients.

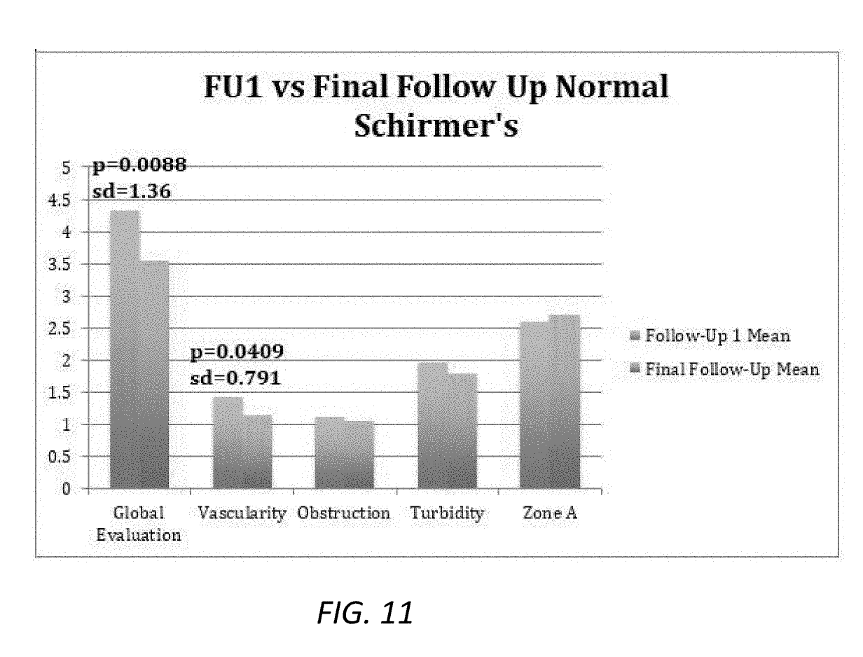

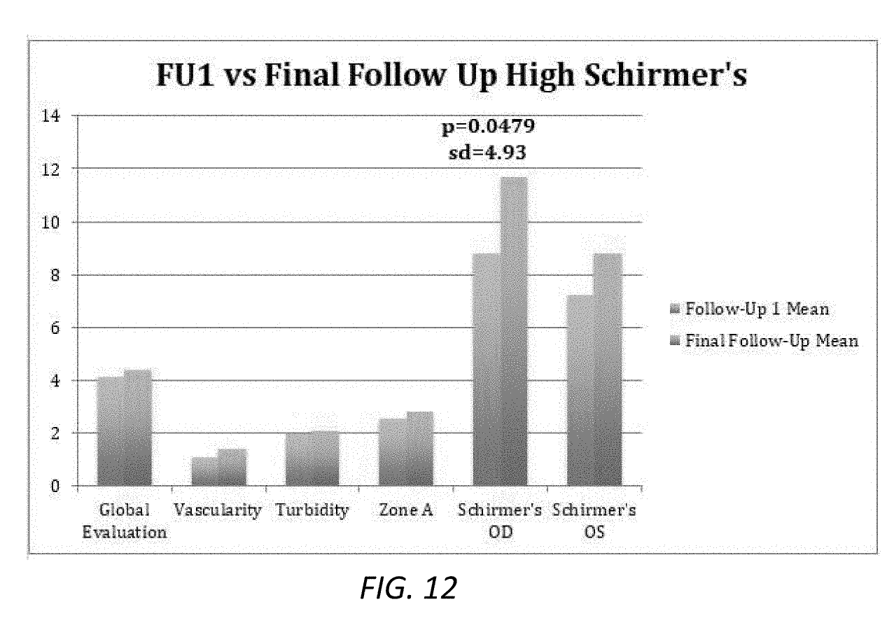

FIG. 11 shows the same statistically significant measurements from FIG. 9 in an attempt to measure the continued effectiveness of the drug on these parameters in patients with normal Schirmer's scores. FIG. 12 similarly shows the same statistically significant measurements from FIG. 10 to measure the continued effect of the drug on these parameters in patients with low Schirmer's scores.

Baseline vs Follow Up 1: Normal Schirmer's Cohort

For the cohort of 75 patients with Schirmer's scores of greater than 5, the mean subjective global assessment score of MGD prior to treatment based on self-reported symptoms was 5.32±2.12. After treatment with spironolactone, the mean subjective global assessment score was 4.03±1.57. The patients had an improvement of 1.29±1.80 (p=0.00) in self-reported global assessment scores.

The mean keratitis and conjunctival staining scores of the right eye prior to treatment were 0.49±0.64, 0.03±0.23, and 0.77±0.71 in the temporal, corneal, and nasal regions respectively. The mean keratitis and conjunctival staining scores of the left eye prior to treatment were 0.64±0.63, 0.07±0.29, and 0.84±0.77 in the temporal, corneal, and nasal regions respectively. Post-treatment mean keratitis and conjunctival scores were 0.45±0.64, 0.007±0.06, and 0.64±0.68 in temporal, corneal, and nasal regions of the right eye and 0.57±0.69, 0.07±0.34, and 0.72±0.73 in the temporal, corneal and nasal regions of the left eye, respectively. The mean change of keratitis and conjunctival scores were −0.03±0.65 (p=0.99), −0.02±0.24 (p=0.99), and −0.13±0.56 (p=0.22) in temporal, corneal, and nasal regions of the right eye and −0.06±0.59 (p=0.43), 0±0.32 (p=0.43), and −0.12±0.67 (p=0.24) in temporal, corneal, and nasal regions of the left eye.

The mean vision of patients prior to treatment was 0.09±0.17 and 0.13±0.23 log Mar units in the right and left eyes, respectively. After treatment, the vision of patients was 0.08±0.18 and 0.11±0.22 log units in the right and left eyes, respectively. The mean change in MAR was −0.016±0.08 log units (p=0.0562) for the right eye and −0.018±0.095 log units (p=0.248) for the left eye.

The mean anterior blepharitis score was 0.35±0.82 prior to treatment and 0.39±0.67 post-treatment; mean change in anterior blepharitis score was 0.04±0.70 (p=0.6475). The mean vascularity score was 1.42±0.897 prior to treatment and 1.11±0.829 post-treatment, with a mean improvement of 0.31±0.713 (p=0.0009). The mean obstruction score was 1.35±0.81 prior to treatment and 0.98±0.69 post-treatment, with a mean improvement of 0.36±0.90 (p=0.0011). The mean turbidity score was 2.68±0.543 prior to treatment and 1.75±0.75 post-treatment, with a mean improvement of 0.93±0.73 (p=0). The mean zone A score was 3.07±0.80 prior to treatment and 2.51±0.88 post-treatment, with a mean improvement of 0.53±0.83 (p=0). The Schirmer's score prior to treatment was 16.31±8.28 and 15.97±7.78 for the right and left eyes respectively. Post treatment, the Schirmer's scores were 16.47±9.75 and 16.95±9.61, with a mean improvement of 0.11±8.07 (p=0.454) and 0.92±9.43 (p=0.204) for the right and left eyes, respectively.

Baseline vs Follow Up 1: Low Schirmer's Cohort

For the cohort of 27 patients with Schirmer's scores of less than 5, the mean subjective global assessment score of MGD prior to treatment based on self-reported symptoms was 4.76±2.37. After treatment with spironolactone, the mean subjective global assessment score was 3.78±1.68. The patients had an improvement of 0.98±2.18 (p=0.0297) in self-reported global assessment scores.

The mean keratitis and conjunctival staining scores of the right eye prior to treatment were 0.94±1.06, 0.20±0.52, and 0.98±0.87 in the temporal, corneal, and nasal regions respectively. The mean keratitis and conjunctival staining scores of the left eye prior to treatment were 0.92±0.93, 0.15±0.37, and 1.04±0.88 in the temporal, corneal, and nasal regions respectively. Post-treatment mean keratitis and conjunctival scores were 0.74±0.91, 0.07±0.38, and 0.93±0.84 in temporal, corneal, and nasal regions of the right eye and 0.96±0.82, 0.15±0.37, and 1.19±0.87 in the temporal, corneal and nasal regions of the left eye, respectively. The mean change of keratitis and conjunctival scores were −0.20±0.75 (p=0.349), −0.13±0.55 (p=0.2991), and −0.056±0.53 (p=0.403) in temporal, corneal, and nasal regions of the right eye and 0.038±0.56 (p=0.743), 0±0.4 (p=1), and 0.15±0.70 (p=0.386) in temporal, corneal, and nasal regions of the left eye.

The mean vision of patients prior to treatment was 0.08±0.14 and 0.15±0.28 log Mar units in the right and left eyes, respectively. After treatment, the vision of patients was 0.05±0.12 and 0.13±0.30 log units in the right and left eyes, respectively. The mean change in MAR was −0.0345±0.08 log units (p=0.0324) for the right eye and −0.0198±0.078 log units (p=0.2112) for the left eye.

The mean anterior blepharitis score was 0.41±0.83 prior to treatment and 0.41±0.71 post-treatment; mean change in anterior blepharitis score was 0.0±0.64 (p=0.7535). The mean vascularity score was 1.26±0.90 prior to treatment and 0.93±0.829 post-treatment, with a mean improvement of 0.33±0.55 (p=0.0056). The mean obstruction score was 1.52±0.88 prior to treatment and 1.13±1.05 post-treatment, with a mean improvement of 0.39±1.48 (p=0.1531). The mean turbidity score was 2.57±0.51 prior to treatment and 1.89±0.91 post-treatment, with a mean improvement of 0.69±0.76 (p=0.0005). The mean zone A score was 3.09±0.92 prior to treatment and 2.46±1.13 post-treatment, with a mean improvement of 0.63±1.21 (p=0.0199). The Schirmer's 1 score prior to treatment was 4.91±4.05 and 4.60±3.73 for the right and left eyes respectively. Post treatment, the Schirmer's scores were 7.52±7.42 and 6.92±5.34, with a mean improvement of 2.61±7.03 (p=0.0324) and 2.33±4.74 (p=0.0096) for the right and left eyes, respectively.

Follow Up 1 vs Final Follow Up: Normal Schirmer's Cohort

For the cohort of 28 patients with Schirmer's scores of greater than 5 and more than 1 follow up, the mean subjective global assessment score of MGD after the first follow up based on self-reported symptoms was 4.32±1.53. After the final follow up treatment with spironolactone, the mean subjective global assessment score was 3.55±1.30. The patients had an improvement of 0.77±1.36 (p=0.0088) in self-reported global assessment scores.

The mean keratitis and conjunctival staining scores of the right eye after the first follow up were 0.54±0.68, 0.0±0.0, and 0.64±0.72 in the temporal, corneal, and nasal regions respectively. The mean keratitis and conjunctival staining scores of the left eye after the first follow up were 0.71±0.80, 0.14±0.52, and 0.82±0.80 in the temporal, corneal, and nasal regions respectively. The final follow up mean keratitis and conjunctival scores were 0.55±0.60, 0.0±0.0, and 0.63±0.63 in temporal, corneal, and nasal regions of the right eye and 0.70±0.57, 0.16±0.49, and 0.75±0.57 in the temporal, corneal and nasal regions of the left eye, respectively. The mean change of keratitis and conjunctival scores were 0.017±0.57 (p=0.69), 0.0±0.0, and −0.018±0.66 (p=0.79) in temporal, corneal, and nasal regions of the right eye and −0.018±0.60 (p=0.89), 0.018±0.21 (p=0.98), and −0.071±0.60 (p=0.89) in temporal, corneal, and nasal regions of the left eye.

The mean vision of patients after the first follow up was 0.07±0.20 and 0.09±0.15 log Mar units in the right and left eyes, respectively. After the final follow up, the vision of patients was 0.05±0.18 and 0.05±0.12 log units in the right and left eyes, respectively. The mean change in MAR was −0.03±0.095 log units (p=0.068) for the right eye and −0.04±0.11 log units (p=0.086) for the left eye

The mean anterior blepharitis score was 0.29±0.50 after the first follow up and 0.32±0.78 after the final follow up; mean change in anterior blepharitis score was 0.04±0.64 (p=0.8046). The mean vascularity score was 1.42±0.84 after the first follow up and 1.13±0.91 post-treatment, with a mean improvement of 0.29±0.79 (p=0.0409). The mean obstruction score was 1.12±0.83 after the first follow up and 1.05±0.71 post-treatment, with a mean improvement of 0.06±0.87 (p=0.66). The mean turbidity score was 1.96±0.62 after the first follow up and 1.80±0.83 post-treatment, with a mean improvement of 0.16±1.00 (p=0.13). The mean zone A score was 2.59±0.82 after the first follow up and 2.71±0.80 post-treatment, with a mean change of 0.13±0.69 (p=0.6142). The Schirmer's score after the first follow up was 17.07±10.10 and 17.50±10.42 for the right and left eyes respectively. Post treatment, the Schirmer's scores were 18.52±9.83 and 17.04±8.48, with a mean change of 1.77±9.01 (p=0.16) and −0.81±7.74 (p=0.71) for the right and left eyes, respectively.

Follow Up 1 vs Final Follow Up: Low Schirmer's Cohort

For the cohort of 10 patients with Schirmer's scores of less than 5 and greater than 1 follow up, the mean subjective global assessment score of MGD after the first follow up based on self-reported symptoms was 4.15±2.07. After the final follow up treatment with spironolactone, the mean subjective global assessment score was 4.4±2.62. The patients had a change of 0.25±2.55 (p=0.92) in self-reported global assessment scores.

The mean keratitis and conjunctival staining scores of the right eye after the first follow up were 0.65±0.71, 0.0±0.0, and 0.60±0.66 in the temporal, corneal, and nasal regions respectively. The mean keratitis and conjunctival staining scores of the left eye after the first follow up were 1.0±0.66, 0.22±0.44, and 1.28±0.57 in the temporal, corneal, and nasal regions respectively. The final follow up mean keratitis and conjunctival scores were 0.7±0.71, 0.0±0.0, and 0.75±0.63 in temporal, corneal, and nasal regions of the right eye and 0.83±0.79, 0.33±0.5, and 1.06±0.92 in the temporal, corneal and nasal regions of the left eye, respectively.

The mean vision of patients after the first follow up was 0.07±0.10 and 0.09±0.14 log Mar units in the right and left eyes, respectively. After the final follow up, the vision of patients was 0.13±0.15 and 0.11±0.11 log units in the right and left eyes, respectively. The mean change in MAR was 0.06±0.10 log units (p=0.12) for the right eye and 0.02±0.07 log units (p=0.45) for the left eye.

The mean anterior blepharitis score was 0.3±0.67 after the first follow up and 0.2±0.42 after the final follow up; mean change in anterior blepharitis score was −0.1±0.57 (p=0.56). The mean vascularity score was 1.1±0.88 after the first follow up and 1.4±0.77 post-treatment, with a mean change of 0.3±0.63 (p=0.19). The mean obstruction score was 1.35±1.06 after the first follow up and 0.75±0.42 post-treatment, with a mean improvement of 0.6±1.13 (p=0.22). The mean turbidity score was 1.95±0.76 after the first follow up and 2.1±0.74 post-treatment, with a mean change of 0.15±0.82 (p=0.57). The mean zone A score was 2.55±0.98 after the first follow up and 2.8±0.79 post-treatment, with a mean change of 0.25±0.75 (p=0.34). The Schirmer's score after the first follow up was 8.8±6.7 and 7.22±5.19 for the right and left eyes respectively. Post treatment, the Schirmer's scores were 11.7±10.94 and 8.78±9.98, with a mean improvement of 2.9±4.93 (p=0.048) and 1.56±6.25 (p=0.238) for the right and left eyes, respectively.

TABLE 1
Pre-treatment Descriptive Statistics for Normal Schirmer's Cohort
(n = 75)

| Parameter | Mean | Standard Deviation |
|---|---|---|
| Subjective Global | 5.32 | 2.123930574 |
| Assessment | | |
| Keratitis OD Temporal | 0.486486486 | 0.640916365 |
| Keratitis OD Corneal | 0.027027027 | 0.232495277 |
| Keratitis OD Nasal | 0.77027027 | 0.708022469 |
| Keratitis OS Nasal | 0.844594595 | 0.767158409 |
| Keratitis OS Corneal | 0.067567568 | 0.290539464 |
| Keratitis OS Temporal | 0.635135135 | 0.631899167 |
| Anterior Blepharitis | 0.346666667 | 0.82188533 |
| Vascularity | 1.42 | 0.896841304 |
| Obstruction | 1.346666667 | 0.813622752 |
| Turbidity | 2.68 | 0.543014608 |
| Zone A | 3.066666667 | 0.802585911 |
| Vision OD (log Mar units) | 0.093424658 | 0.169162473 |
| Vision OS (log Mar units) | 0.128506849 | 0.231239602 |
| Schirmer's OD (mm) | 16.31081081 | 8.275935863 |
| Schirmer's OS (mm) | 15.97297297 | 7.765347989 |

TABLE 2
Pre-treatment Descriptive Statistics for Low Schirmer's Cohort
(n = 27)

| Parameter | Mean | Standard Deviation |
|---|---|---|
| Subjective Global | 4.759259259 | 2.367124068 |
| Assessment | | |
| Keratitis OD Temporal | 0.944444444 | 1.059148182 |
| Keratitis OD Corneal | 0.203703704 | 0.523656876 |
| Keratitis OD Nasal | 0.981481481 | 0.871354841 |
| Keratitis OS Nasal | 1.038461538 | 0.882304674 |
| Keratitis OS Corneal | 0.153846154 | 0.367946484 |
| Keratitis OS Temporal | 0.923076923 | 0.934797387 |
| Anterior Blepharitis | 0.407407407 | 0.832478194 |
| Vascularity | 1.259259259 | 0.902670934 |
| Obstruction | 1.518518519 | 0.882320819 |
| Turbidity | 2.574074074 | 0.513354423 |
| Zone A | 3.092592593 | 0.920253279 |
| Vision OD (log Mar units) | 0.081346154 | 0.142363743 |
| Vision OS (log Mar units) | 0.15052 | 0.282601769 |
| Schirmer's OD (mm) | 4.907407407 | 4.0478794 |
| Schirmer's OS (mm) | 4.596153846 | 3.72563882 |

TABLE 3
Post-treatment Follow-up 1 Statistics - Normal Schirmer's (n = 75)

| Parameter | Mean | Standard Deviation |
|---|---|---|
| Subjective Global | 4.033333333 | 1.573284852 |
| Assessment | | |
| Keratitis OD Temporal | 0.452702703 | 0.636605504 |
| Keratitis OD Corneal | 0.006756757 | 0.058123819 |
| Keratitis OD Nasal | 0.641891892 | 0.680049707 |
| Keratitis OS Nasal | 0.722972973 | 0.731586248 |
| Keratitis OS Corneal | 0.067567568 | 0.344470254 |
| Keratitis OS Temporal | 0.574324324 | 0.690852325 |
| Anterior Blepharitis | 0.386666667 | 0.670686081 |
| Vascularity | 1.11 | 0.82940063 |
| Obstruction | 0.983333333 | 0.690589903 |
| Turbidity | 1.753333333 | 0.75055535 |
| Zone A | 2.513333333 | 0.881389157 |
| Vision OD (log Mar units) | 0.076861111 | 0.177712246 |
| Vision OS (log Mar units) | 0.111486111 | 0.224335011 |
| Schirmer's OD (mm) | 16.46575342 | 9.752609611 |
| Schirmer's OS (mm) | 16.94520548 | 9.61175555 |

TABLE 4
Post-treatment Follow-up 1 Statistics - Low Schirmer's (n = 27)

| Parameter | Mean | Standard Deviation |
|---|---|---|
| Subjective Global | 3.777777778 | 1.683250823 |
| Assessment | | |
| Keratitis OD Temporal | 0.740740741 | 0.913260962 |
| Keratitis OD Corneal | 0.074074074 | 0.384900179 |
| Keratitis OD Nasal | 0.925925926 | 0.840143116 |
| Keratitis OS Nasal | 1.192307692 | 0.872661711 |
| Keratitis OS Corneal | 0.153846154 | 0.367946484 |
| Keratitis OS Temporal | 0.961538462 | 0.823687768 |
| Anterior Blepharitis | 0.407407407 | 0.707610239 |
| Vascularity | 0.925925926 | 0.828619144 |
| Obstruction | 1.12962963 | 1.052401947 |
| Turbidity | 1.888888889 | 0.912870929 |
| Zone A | 2.462962963 | 1.134476548 |
| Vision OD (log Mar units) | 0.046807692 | 0.117415849 |
| Vision OS (log Mar units) | 0.13072 | 0.300851226 |
| Schirmer's OD (mm) | 7.518518519 | 7.418119036 |
| Schirmer's OS (mm) | 6.923076923 | 5.336089032 |

TABLE 5
Baseline vs. Follow up 1 statistics - Normal Schirmer's (n = 75)

| Parameter | Mean | Standard Deviation | p-value |
|---|---|---|---|
| Subjective Global | −1.286666667 | 1.797470695 | 0 |
| Assessment | | | |
| Keratitis OD Temporal | −0.033333333 | 0.654162004 | 0.9929 |
| Keratitis OD Corneal | −0.02027027 | 0.240421849 | 0.9923 |
| Keratitis OD Nasal | −0.126666667 | 0.564090594 | 0.2187 |
| Keratitis OS Nasal | −0.121621622 | 0.671385864 | 0.2412 |
| Keratitis OS Corneal | 0 | 0.320530382 | 0.4342 |
| Keratitis OS Temporal | −0.060810811 | 0.590754699 | 0.4347 |
| Anterior Blepharitis | 0.04 | 0.701157344 | 0.6475 |
| Vascularity | −0.31 | 0.712864416 | 0.0009 |
| Obstruction | −0.363333333 | 0.901662628 | 0.0011 |
| Turbidity | −0.926666667 | 0.72918565 | 0 |
| Zone A | −0.553333333 | 0.832504092 | 0 |
| Vision OD | −0.015633803 | 0.080920638 | 0.0562 |
| (log Mar units) | | | |
| Vision OS | −0.017957746 | 0.095495017 | 0.2483 |
| (log Mar units) | | | |
| Schirmer's OD (mm) | 0.109589041 | 8.071833132 | 0.454 |
| Schirmer's OS (mm) | 0.917808219 | 9.436562205 | 0.2044 |

TABLE 6
Baseline vs. Follow up 1 statistics - Low Schirmer's (n = 27)

| Parameter | Mean | Standard Deviation | p-value |
|---|---|---|---|
| Subjective Global | −0.981481481 | 2.177160649 | 0.0297 |
| Assessment | | | |
| Keratitis OD Temporal | −0.203703704 | 0.750118699 | 0.3493 |
| Keratitis OD Corneal | −0.12962963 | 0.547592503 | 0.2991 |
| Keratitis OD Nasal | −0.055555556 | 0.525015262 | 0.403 |
| Keratitis OS Nasal | 0.153846154 | 0.703835645 | 0.3862 |
| Keratitis OS Corneal | 0 | 0.4 | 1 |
| Keratitis OS Temporal | 0.038461538 | 0.564323966 | 0.7432 |
| Anterior Blepharitis | 0 | 0.635488909 | 0.7535 |
| Vascularity | −0.333333333 | 0.554700196 | 0.0056 |
| Obstruction | −0.388888889 | 1.476308633 | 0.1531 |
| Turbidity | −0.685185185 | 0.761427659 | 0.0005 |
| Zone A | −0.62962963 | 1.205696356 | 0.0199 |
| Vision OD (log Mar | −0.034538462 | 0.077540302 | 0.0274 |
| units) | | | |
| Vision OS (log Mar | −0.0198 | 0.078135566 | 0.2112 |
| units) | | | |
| Schirmer's OD (mm) | 2.611111111 | 7.033345485 | 0.0324 |
| Schirmer's OS (mm) | 2.326923077 | 4.739076509 | 0.0096 |

TABLE 7
Follow-up 1 - patients with >1 follow-ups -
Normal Schirmer's (n = 28)

| Parameter | Mean | Standard Deviation |
|---|---|---|
| Global Evaluation | 4.321428571 | 1.528823596 |
| Keratitis OD Temporal | 0.535714286 | 0.679441102 |
| Keratitis OD Corneal | 0 | 0 |
| Keratitis OD Nasal | 0.642857143 | 0.718243006 |
| Keratitis OS Nasal | 0.821428571 | 0.79598862 |
| Keratitis OS Corneal | 0.142857143 | 0.524530528 |
| Keratitis OS Temporal | 0.714285714 | 0.798477387 |
| Anterior Blepharitis | 0.285714286 | 0.498675494 |
| Vascularity | 1.419642857 | 0.841772742 |
| Obstruction | 1.116071429 | 0.831893598 |
| Turbidity | 1.964285714 | 0.622548101 |
| Zone A | 2.589285714 | 0.817103867 |
| Vision OD | 0.07437037 | 0.200595296 |
| Vision OS | 0.088259259 | 0.148777478 |
| Schirmer's OD | 17.07142857 | 10.10290963 |
| Schirmer's OS | 17.5 | 10.41899974 |

TABLE 8
Follow-up 1 statistics for patients with >1 follow-ups - Low Schirmer's
(n = 10)

| Parameter | Mean | Standard Deviation |
|---|---|---|
| Global Evaluation | 4.15 | 2.068950351 |
| Keratitis OD Temporal | 0.65 | 0.709068246 |
| Keratitis OD Corneal | 0 | 0 |
| Keratitis OD Nasal | 0.6 | 0.658280589 |
| Keratitis OS Nasal | 1.277777778 | 0.565194165 |
| Keratitis OS Corneal | 0.222222222 | 0.440958552 |
| Keratitis OS Temporal | 1 | 0.661437828 |
| Anterior Blepharitis | 0.3 | 0.674948558 |
| Vascularity | 1.1 | 0.875595036 |
| Obstruction | 1.35 | 1.055409347 |
| Turbidity | 1.95 | 0.761941963 |
| Zone A | 2.55 | 0.984603699 |
| Vision OD | 0.066333333 | 0.104743735 |
| Vision OS | 0.087375 | 0.136033544 |
| Schirmer's OD | 8.8 | 6.6633325 |
| Schirmer's OS | 7.222222222 | 5.190803834 |

TABLE 9
Final Follow-up - patients with >1 follow-ups -
Normal Schirmer's (n = 28)

| Parameter | Mean | Standard Deviation |
|---|---|---|
| Global Evaluation | 3.553571429 | 1.30056459 |
| Keratitis OD Temporal | 0.553571429 | 0.598443749 |
| Keratitis OD Corneal | 0 | 0 |
| Keratitis OD Nasal | 0.625 | 0.632821431 |
| Keratitis OS Nasal | 0.75 | 0.569275043 |
| Keratitis OS Corneal | 0.160714286 | 0.491663304 |
| Keratitis OS Temporal | 0.696428571 | 0.566654995 |
| Anterior Blepharitis | 0.321428571 | 0.784269957 |
| Vascularity | 1.125 | 0.909059343 |
| Obstruction | 1.053571429 | 0.711535712 |
| Turbidity | 1.803571429 | 0.831545702 |
| Zone A | 2.714285714 | 0.798477387 |
| Vision OD | 0.045925926 | 0.181355988 |
| Vision OS | 0.049333333 | 0.117930553 |
| Schirmer's OD | 18.51851852 | 9.830761651 |
| Schirmer's OS | 17.03703704 | 8.482930741 |

TABLE 10
Final Follow-up - patients with >1 follow-ups -
Low Schirmer's (n = 10)

| Parameter | Mean | Standard Deviation |
|---|---|---|
| Global Evaluation | 4.4 | 2.622551768 |
| Keratitis OD Temporal | 0.7 | 0.714920353 |
| Keratitis OD Corneal | 0 | 0 |
| Keratitis OD Nasal | 0.75 | 0.634647759 |
| Keratitis OS Nasal | 1.055555556 | 0.916666667 |
| Keratitis OS Corneal | 0.333333333 | 0.5 |
| Keratitis OS Temporal | 0.833333333 | 0.790569415 |
| Anterior Blepharitis | 0.2 | 0.421637021 |
| Vascularity | 1.4 | 0.774596669 |
| Obstruction | 0.75 | 0.424918293 |
| Turbidity | 2.1 | 0.737864787 |
| Zone A | 2.8 | 0.788810638 |
| Vision OD | 0.126444444 | 0.153845792 |
| Vision OS | 0.105875 | 0.108030998 |
| Schirmer's OD | 11.7 | 10.94481105 |
| Schirmer's OS | 8.777777778 | 9.975316759 |

TABLE 11
Follow-up 1 vs Final Follow-up statistics -
Normal Schirmer's (n = 28)

| Parameter | Mean | Standard Deviation | p-value |
|---|---|---|---|
| Global Evaluation | −0.767857143 | 1.364104257 | 0.0088 |
| Keratitis OD Temporal | 0.017857143 | 0.568984522 | 0.6936 |
| Keratitis OD Corneal | 0 | 0 | |
| Keratitis OD Nasal | −0.017857143 | 0.659434983 | 0.7937 |
| Keratitis OS Nasal | −0.071428571 | 0.604217978 | 0.8857 |
| Keratitis OS Corneal | 0.017857143 | 0.214395915 | 0.9795 |
| Keratitis OS Temporal | −0.017857143 | 0.600650001 | 0.8863 |
| Anterior Blepharitis | 0.035714286 | 0.63724772 | 0.8046 |
| Vascularity | −0.294642857 | 0.790726258 | 0.0409 |
| Obstruction | −0.0625 | 0.865022479 | 0.66 |
| Turbidity | −0.160714286 | 1.000495909 | 0.1296 |
| Zone A | 0.125 | 0.688866487 | 0.6142 |
| Vision OD | −0.033269231 | 0.094566615 | 0.0679 |
| Vision OS | −0.040423077 | 0.111040956 | 0.0861 |
| Schirmer's OD | 1.777777778 | 9.014233759 | 0.1575 |
| Schirmer's OS | −0.814814815 | 7.741183755 | 0.7055 |

TABLE 12
Follow-up 1 vs Final Follow-up statistics - Low Schirmer's (n = 10)

| Parameter | Mean | Standard Deviation | p-value |
|---|---|---|---|
| Global Evaluation | 0.25 | 2.552232139 | 0.9185 |
| Keratitis OD Temporal | 0.05 | 0.643341969 | 0.4138 |
| Keratitis OD Corneal | 0 | 0 | |
| Keratitis OD Nasal | 0.15 | 0.411636301 | 0.2922 |
| Keratitis OS Nasal | −0.222222222 | 0.833333333 | 0.713 |
| Keratitis OS Corneal | 0.111111111 | 0.333333333 | 0.3173 |
| Keratitis OS Temporal | −0.166666667 | 0.559016994 | 0.4888 |
| Anterior Blepharitis | −0.1 | 0.567646212 | 0.5637 |
| Vascularity | 0.3 | 0.632455532 | 0.1882 |
| Obstruction | −0.6 | 1.125462868 | 0.2215 |
| Turbidity | 0.15 | 0.818195847 | 0.569 |
| Zone A | 0.25 | 0.754615428 | 0.3377 |
| Vision OD | 0.060111111 | 0.102126691 | 0.1171 |
| Vision OS | 0.0185 | 0.07362647 | 0.4477 |
| Schirmer's OD | 2.9 | 4.931756505 | 0.0479 |
| Schirmer's OS | 1.555555556 | 6.252221827 | 0.2384 |

Discussion

This study is the first to evaluate the potential of topical spironolactone to treat MGD. Based on the results shown in Table 3 and FIG. 9, application of a topical spironolactone ophthalmic suspension has a statistically significant effect in patients with Schirmer's test scores of greater than 5 (p<0.05) on the subjective global assessment score, vascularity score, obstruction score, turbidity grade, and the zone A grade. The change in global evaluation score (p=0.0) suggests that patients' symptoms associated with dry eye syndrome (redness, swelling, and irritation) decreased. The most statistically significant changes were in the global evaluation, as mentioned above, as well as in the turbidity and zone A scores. This change in turbidity (p=0.0) suggests that spironolactone may have a role in changing the quality of the expressed meibum through various mechanisms based on its diverse pharmacological properties. In addition, the change in zone A grade (p=0.0) may reflect the anti-inflammatory properties of spironolactone. In addition, although the vision did not show statistically significant improvement, both eyes did seem to show improvement (p=0.0562 and p=0.2483, respectively) on the log mar scale, which might suggest improved vision due to the decreased dry eye symptoms that caused blurriness.

Based on the results shown in Table 6 and FIG. 10, application of topical spironolactone ophthalmic suspension also has a statistically significant effect in patients with Schirmer's test scores of 5 or less (p<0.05) on the subjective global assessment score, vascularity score, turbidity score, zone A score, Schirmer's score in both eyes, as well as the vision log Mar score in the right eye. Similar to the cohort with Schirmer's scores of greater than 5, the significant improvement in turbidity (p=0.0005) and zone A (p=0.0199) suggest the effectiveness of spironolactone in increasing the quality of the meibum, as well decreasing inflammation. In addition, one interesting outcome measured was the increase in Schirmer's tear test scores in both eyes in this cohort. This finding suggests that spironolactone may play a role in increasing the quantity of tears produced, along with the increased quality of the meibum as mentioned earlier. The statistically significant improvement in the vision of the right eye (p=0.0274) also suggests improvement of the dry eye symptoms, which would possibly lead to less blurred vision. Although the left eye did not show statistically significant improvement as the right eye did, there was still measured improvement in vision (p=0.2112). A larger sample size of patients with low Schirmer's scores might help reduce these discrepancies.

In the 28 patients who had Schirmer's scores of greater than 5, and who also had more than 1 follow up, there was a statistically significant improvement in the global evaluation (p=0.0) and the vascularity (p=0.0409) scores as seen in table 11 and FIG. 11. This suggests that patients continued to feel an improvement in their dry eye symptoms, such as redness, swelling, and irritation even after the initial follow up. Further, although many of the previously statistically significant parameters, such as obstruction, turbidity, and zone A scores, did not show continued statistically significant improvement, many did still show overall improvement, as seen in table 11 and FIG. 11. For example, turbidity continued to show improvement, which suggests increased quality of meibum secretions when using spironolactone over time. Ultimately, these findings warrant further study of the effectiveness of spironolactone over sustained periods of time. With a larger sample size, observation of the changes in the aforementioned parameters would likely be more informative regarding the requirement for long-term use of spironolactone.

In the 10 patients with multiple follow ups and with Schirmer's scores of 5 or fewer, the only statistically significant improvement was in Schirmer's scores in the right eye (p=0.0479) as seen in table 12 and FIG. 12. This further suggests improvement in the quantity of tears after using Spironolactone. However, the other previously statistically significant improved measurements, including global evaluation, vascularity, turbidity and zone A scores slightly worsened, though not to a statistically significant extent. However, due to the extremely small sample size of 10 patients in this cohort, it is difficult to make any definitive conclusions regarding the long-term effectiveness of the drug in this cohort. Therefore, as in the previous cohort, it would likely be necessary and informative to study the long-term effectiveness of Spironolactone in a larger sample size of patients with low Schirmer's test scores.

Spironolactone was first used as a potassium-sparing diuretic due to its antagonistic activity at aldosterone receptors. Many other properties of spironolactone have been discovered including its activity at testosterone receptors. More specifically, Spironolactone has been found to have both partial agonistic and antagonistic activities at testosterone receptors (see Térouanne B, Tahiri B, Georget V, et al. (February 2000). “A stable prostatic bioluminescent cell line to investigate androgen and antiandrogen effects”. Molecular and Cellular Endocrinology 160 (1-2): 39-49. doi:10.1016/S0303-7207(99)00251-8).

Due to the antagonistic properties, spironolactone has been used off-label for the treatment of hormonal acne in women and to suppress unwanted effects of androgens in individuals undergoing gender reassignment. Since testosterone has a known role in the development and function of meibomian glands, it is reasonable to assume that spironolactone may have additional off-label uses and potentially benefit patients with MGD.

MGD has been found to be present in both patients with low testosterone levels as well as high testosterone levels. The presence of MGD in patients with low testosterone levels was identified in 2002 and subsequent studies have confirmed the influence of androgens on gene regulation in meibomian glands. For example, anti-androgen therapy in men and androgen receptor dysfunction in women led to the symptoms of MGD and evaporative dry eye. Furthermore, it was found that this androgen insufficiency could play a role in MGD (see Sullivan D A, Sullivan B D, Evans J E, et al. Androgen deficiency, Meibomian gland dysfunction, and Evaporative dry eye. Ann N Y Acad Sci. 2002; 966:211-222). Similarly, a study in 2006 found that patients with MGD had a depleted androgen pool compared to non-MGD patients. This decreased androgen pool was hypothesized to alter the lipid metabolic pathways that influence the meibomian glands. See Tamer C, Oksuz H, Sogut S, Androgen status of the nonautoimmune dry eye subtypes, Ophthalmic Res. 2006; 38(5): 280-6, Epub 2006 Sep. 15. In addition, a study in 2013 showed that testosterone plays a role in down-regulating the expression of genes that cause keratinization of Meibomian Glands, which has the potential to prevent MGD. See Schirra F, Gatzioufas Z, Scheidt J, Seitz B, Testosterone reduces the expression of keratinization-promoting genes in murine Meibomian glands, Ophthalmologe, 2013 March; 110(3): 230-8, doi: 10.1007/s00347-012-2661-5 (article in German). On the other hand, a study in 2011, showed that levels of testosterone were elevated in patients with MGD and suggested that elevated levels of testosterone should be used as a diagnostic criteria for diagnosing MGD (see Ozlem G. Sahin, ElçinKartal, and NusretTaheri, “Meibomian Gland Dysfunction: Endocrine Aspects,” ISRN Ophthalmology, vol. 2011, Article ID 465198, 6 pages, 2011. doi:10.5402/2011/465198). After an improvement in the understanding of MGD, one explanation of these conflicting results is that Sullivan was studying hyposecretory states resulting in MGD, while Sahin was studying hypersecretory states resulting in MGD. This suggests that it is necessary to have a proper balance of testosterone levels for meibomian glands to secrete meibum with an optimal quality for maintaining tear film. Spironolactone's property as both a weak partial agonist and an antagonist of testosterone may help maintain a balanced level of testosterone. This property of spironolactone may address the underlying cause of MGD pathogenesis in addition to addressing the symptoms that may be associated with its anti-inflammatory properties.

An advantage of using topical spironolactone, rather than oral spironolactone, is that a lower concentration is necessary to deliver an effective dose directly to the site of action. Topical use of spironolactone still has possible side effects. A small percentage of the patients reported a mild temporary burning sensation in the eye after administration of spironolactone. The role of the anti-aldosterone activity of spironolactone in the eye is unclear. The presence of a renin-angiotensin system has been identified and has been a potential target for lowering intraocular pressure in patients with glaucoma (see Strain W D, Chaturvedi N. The renin-angiotensin-aldosterone system and the eye in diabetes. J Renin Angiotensin Aldosterone Syst. 2002; 3:243-246).

Limitations of this study include the retrospective nature of this first pilot study, the lack of a control group, and the small number of patients, especially in the low Schirmer's cohort with at least 1 follow up (n=27), the normal Schirmer's cohort with multiple follow ups (n=28), and the low Schirmer's cohort with multiple follow ups (n=10). Future prospective masked placebo controlled studies of MGD are obviously warranted.

### Example 9

### RT-PCR Results Measuring ELOVL4 Gene Expression in Corneal Epithelial (Human Telomerase-Immortalized Corneal Epithelial (HTCE)) Cells Treated with Different Dilutions of a 0.025 mg/mL Spironolactone Formulation

HTCE cells were cultured in 6 well plates and grown to about 30% confluence. The cells were then treated with different dilutions of a 0.025 mg/ml Spironolactone eye drop formulation (i.e. 50×, 100×, 500×, 1000×, and 5000×, corresponding to 0.0005 mg/ml, 0.00025 mg/ml, 0.00005 mg/ml, 0.000025 mg/ml, and 0.000005 mg/ml). The medium with different concentrations of Spironolactone was changed every 2 days. On the 7th day, the cells were collected, and the RNA was extracted and subject to RT-PCR analysis for gene expression of ELOVL4, an enzyme involved in in the biosynthesis of fatty acids (particularly very long chain fatty acids (VLCFAs)).

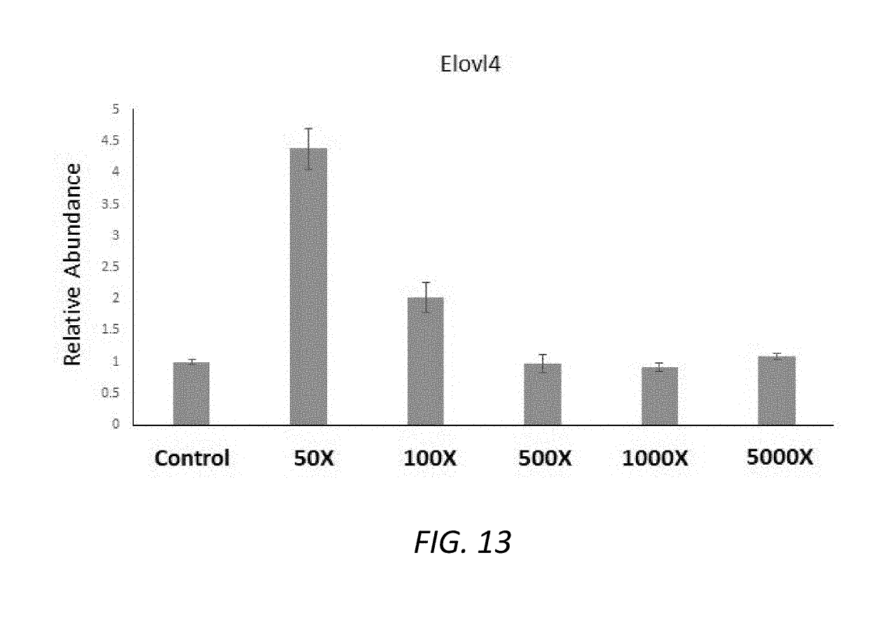

The results (after normalization) are shown in the bar graph in FIG. 13. As the graph shows, ELOVL4 gene expression was upregulated the most in the 0.0005 mg/ml treatment group, with an induction of approximately 4.5-fold. ELOVL4 was induced approximately 2-fold in the 0.00025 mg/ml treatment group. The remaining treatment groups showed no change relative to control.

As a result of increased gene expression and resulting protein expression, the 0.0005 mg/cc concentration of spironolactone would be expected to increase production of VLCFAs produced by the ELOVL4 protein in corneal epithelial cells. These fatty acids would then replace/supplement the oils not being produced by the Meibomian glands in a dry eye situation. Replacement/supplementation of these lipids also increases the quality of the lipids by substituting/replacing the lipids normally generated with different higher-quality lipids.

### Example 10

### Aldosterone Antagonist Representative Compositions

(A) 0.0000025-0.1 mg/cc aldosterone antagonist, with one or more of up to 30% of a pluronic component, such as Pluronic F127, Pluronic F123, Pluronic F127, Pluronic P85, and/or Pluronic F68, up to 30% of a glycol or polyol, such as polyethylene glycol (PEG), PEG 200, PEG 300 and/or PEG 400, and/or up to 10% EDTA, with the balance provided by one or more of water, pyrogen-free water, isotonic saline, phosphate buffer, sodium phosphate dibasic, sodium phosphate monobasic, magnesium hydroxide, aluminum hydroxide, potassium chloride, citric acid, alginic acid, edetate disodium, edetate calcium disodium, edetate sodium, edetate trisodium, edetate dipotassium, sodium phosphate, sodium dihydrogen phosphate, disodium hydrogen phosphate, potassium phosphate, potassium dihydrogen phosphate and dipotassium hydrogen phosphate, sodium borate, potassium borate, sodium citrate, disodium citrate, sodium acetate, potassium acetate, sodium carbonate, sodium hydrogen carbonate, p-hydroxy benzoate esters, benzalkonium chloride, benzethonium chloride, sodium chloride, esters of parahydroxybenzoates (parabens), phenylmercuric acetate, phenylmercuric nitrate, thimerosal, Polyquad, Purite, chlorobutanol, benzyl alcohol, phenylethylalcohol, sorbic acid, chlorhexidine gluconate, sodium dehydroacetate, cetylpyridinium chloride, alkyldiaminoethylglycine hydrochloride, oleic acid, 1-methyl-2 pyrrolidone, 2,2-dimethyl octanoic acid, N,N dimethyl lauramide/propylene glycol monolaureate, and/or dapsone.

(B) Composition (A) with the aldosterone antagonist being one or more of spironolactone, eplerenone, canrenone, prorenone, and/or mexrenone.

(C) Composition (A) or (B) with 0.000005 mg/cc aldosterone antagonist.

(D) Composition (A) or (B) with 0.00005 mg/cc aldosterone antagonist.

(E) Composition (A) or (B) with 0.0005 mg/cc aldosterone antagonist.

(F) Composition (A) or (B) with 0.005 mg/cc aldosterone antagonist.

(G) Composition (A) or (B) with 0.05 mg/cc aldosterone antagonist.

(H) Composition (A) or (B) with 0.025 mg/cc aldosterone antagonist.

(I) Composition (A) or (B) with 0.1 mg/cc aldosterone antagonist.

(J) Composition (A)-(I) with 80-99% water.

(K) Composition (A)-(J) with 1-10% pluronic, 5-20% pluronic, 2-30% pluronic, 2.5-15% pluronic, 8-25% pluronic, or 12-15% pluronic.

(L) Composition (A)-(K) with 0-5% PEG, or 1-10% PEG, or 2-15% PEG, or 3-18% PEG, or 4-12% PEG, or 8-30% PEG, or 20-25% PEG.

(M) Composition (A)-(L) with 0.01-5% EDTA, 0.04-8% EDTA, 0.08-2% EDTA, 0.12-3% EDTA, 0.14-4% EDTA, 0.16-10% EDTA, 0.2-0.5% EDTA, 0.3-0.9% EDTA, 0.06-4.5% EDTA, 1.5-3.5% EDTA, 2.5-7% EDTA, 6-9% EDTA.

According to the literature, Spironolactone is practically insoluble in water. The present inventors were able to get spironolactone into solution at 0.015 mg/ml with the help of Pluronic F127.

### Example 11

### Gene and Cell Therapy for MGD

Meibomian glands are specialized sebaceous glands in the eyelids that are responsible for producing meibum. Meibum is an oily substance that forms the outermost layer of the tear film slowing its evaporation. In humans, there are approximately 30-40 meibomian glands present in the upper eyelid, and approximately 25-30 meibomian glands in the lower eyelid.

Meibomian Gland Dysfunction (MGD) also known as posterior blepharitis is generally described as a chronic, diffuse abnormality of the meibomian glands located in the eyelids. MGD may result in eyelid alterations and is also associated with an alteration of the tear film causing ocular surface disease.

These glands normally release an oily secretion to maintain tear film stability. The meibomian glands of affected individuals are often characterized by terminal duct obstruction, glandular secretion changes, and posterior lid margin inflammation. MGD has been diagnosed based on presence of glandular dropout, reduced secretion upon gland expression, meibum secretion quality, inflammation, and meibography. Arita recommends physicians to suspect obstructive MGD with two or more abnormal scores in ocular symptoms, lid margin abnormalities, and meibo-score.

A group of experts on meibomian glands convened to better define the disease in an effort to better understand the known physiology and pathophysiology and treatment of this prevalent disease.

The incidence of the disease increases with age, and occurs in greater than 80% of patients with complaints of ocular surface disease. A conservative estimate of the number of patients affected with this condition is 20-30 million in the United States alone. Patients with significant disease can be severely impaired causing significant pain, suffering and predisposition to eye threatening visual loss.

Common complaints of patients suffering from meibomian gland disease include blurred or filmy vision, burning or foreign body sensations in the eye, photophobia, and pain severe enough to awaken the person from sleep. Although patients with this condition usually have normal production of aqueous tears by their lacrimal glands, their meibomian glands can atrophy and this is frequently accompanied by metaplasia of the ductal epithelium of these glands. Anterior erosion of the mucocutaneous junction of the eyelid is often noted, as well as eyelid and conjunctival infection, eyelid margin irregularity, corneal epithelial changes, and corneal vascularization.

The mechanisms responsible for the eyelid and ocular surface changes and irritation symptoms experienced by patients with meibomian gland disease are mostly unknown. Therefore, previous treatments of meibomian gland disease were directed only to treatment of presumed infection of the eyelids or meibomian glands, or had particular disadvantages that made such treatments of little use for long periods of time.

Current treatments for MGD include warm compresses, lid hygiene, intraductal meibomian gland probing, lipid-emulsion eye drops, thermal pulsation, n-acetyl-cysteine, azithromycin, omega 3-fatty acid supplementation, cyclosporine A eye drops, and intense pulse-light therapy (IPL). While current treatments target symptoms, they are sometimes poorly tolerated and often offer only temporary relief.

For example, patients with meibomian gland disease have been symptomatically treated with artificial tears, but these agents provide limited, if any, improvement. Topically applied steroids and non-steroidals to the eyelids or ocular surface are effective as short-term pulse therapies. However, steroids are not good long-term solutions because of the potential side-effects e.g., cataract and glaucoma and non-steroidals are fraught with concerns of toxicity, keratitis and corneal melting. Meibomian Gland Disease is currently not curable or reversible; therefore, patients with this condition must be treated for life. Restasis (Allergan) and Xidra (Shire) are commercially available for dry eye indications by improving symptoms, Schirmer scores and surface keratitis. They have not been indicated for MGD based on FDA labeling for both prescriptions.

Orally administered tetracyclines and tetracycline analogues (e.g., doxycycline and minocycline) having antibiotic activity are commonly and effectively used for prophylactic or therapeutic treatment of Meibomian Gland Disease. The mechanism by which tetracyclines work in treating Meibomian Gland Disease is not known (tetracycline is known to chelate Zn++ which inhibits MMP (matrix metalloproteinase) activity), but some relief of symptoms has been reported. However, one disadvantage for using antimicrobially active tetracyclines or tetracycline analogues orally in the treatment of Meibomian Gland Disease is that a high percentage of patients are unable to tolerate oral tetracyclines for extended periods of time. The intolerance to tetracyclines can manifest itself in gastrointestinal problems, e.g., epigastric pain, nausea, vomiting, and diarrhea, or other problems related to taking long-term oral antibiotics, such as mucosal candidiasis. At the present time there are no available long-term treatments of Meibomian Gland Disease.

MGD is thought to be a significant cause of ocular surface disease throughout the world (Nichols et al, Invest. Ophthalmol. Vis. Set 52: 1922-1929, 2011). Over the last 20 years dry eye experts have not always been in agreement with the definition of dry eye and recently have redefined the definition of dry eye and its many treatments. Most dry eye treatments currently available address ocular inflammation, improving Schirmer's scores, and other signs of dry eye, such as keratitis.

The inventors' method not only demonstrates improvement of the ocular surface but more distinctly directly addresses the lipid production abnormalities of posterior blepharitis and an eyelid abnormality associated with MGD.

Recently, spironolactone and other aldosterone inhibiting analogues have been reported to improve patients who have not readily responded to conventional therapies for MGD and ocular surface diseases. This includes even some of the post LASIK corneal neuropathies.

Spironolactone is a drug that has been used as a potassium-sparing diuretic to treat heart failure for over 35 years. In recent years, its anti-androgenic effects have been used in dermatologic settings to treat hirsutism, female pattern hair loss, and hormonal acne in women. Spironolactone is a synthetic 17-lactone steroid with anti-androgenic and anti-inflammatory properties in addition to its anti-hypertensive effect. Its anti-androgenic effect is due to a number of mechanisms including regulating androgen receptors. Unlike anti-androgenic drugs that are testosterone receptor antagonists, spironolactone is unique in that it is a weak partial agonist at androgen receptors and may have an agonistic or antagonistic effect at androgen receptors, depending on the concentration of testosterone. In individuals with normal testosterone levels, spironolactone inhibits key enzymes (17α-hydroxylase) in the androgen biosynthetic pathway, activates the progesterone receptor, and inhibits 5α-reductase, a key enzyme in the synthesis of dihydrotestosterone (DHT), a potent androgen. Due to spironolactone's dual anti- and pro-testosterone properties, anti-aldosterone, and anti-inflammatory properties, it is reasonable to believe that spironolactone may improve the quality of meibomian gland secretions and address the inflammation seen in patients with MGD.

Spironolactone has been associated with adverse effects such as increased urinary frequency, hyperkalemia, rashes, and menstrual irregularities in women. Adverse effects tend to be dose-related, however, the long-term use of spironolactone appears to be safe. The use of spironolactone in an ocular vehicle has never been previously reported in the literature, but may reduce the risk for adverse effects while improving the quality of meibomian gland secretions in MGD by potentially decreasing systemic levels when compared to oral therapy. This is due in large part to a significantly lower dose required when instilling the drug at the site of action. To the best of the inventors' knowledge, the effect of topical spironolactone on MGD has never been studied or previously reported.

A topical eye drop used for any chronic eye condition such as MGD should have qualities of effectiveness without any untoward side effects to the ocular surface such as a drug related toxicity after long periods of use. The inventors' studies suggest topical spironolactone is effective in treating the signs and symptoms of MGD based on 2 retrospective studies demonstrating statistical significant improvement in ocular surface disease based on subjective global assessment scores of symptom relief, keratitis (KS) conjunctival scores (CS), lid margin vascularity grade (V), obstruction grade (O), turbidity grade (T), zone A grade, and vision. In addition, the study shows topical spironolactone improves Schirmer's score in patients with MGD with aqueous tear deficiency.

Certain modifications of the composition of and formulation of topical spironolactone results in an optimized topical composition and method to treat signs and symptoms of MGD without causing long term toxicity. These non-toxic, optimized low dose concentrations of spironolactone are shown to improve the chronic eye conditions associated with MGD.

Elongation of very long chain fatty acids protein 4 is a protein that in humans is encoded by the ELOVL4 gene. ELOVL4 is required for the synthesis of very long chain saturated fatty acids and very long chain polyunsaturated fatty acids, the latter of which are uniquely present in retina, sperm, and brain (Agbaga et al., 2008).

The inventors' in vitro studies using human telomerase-immortalized corneal epithelial cells (HTCE) show maximal upregulation of the ELOVL4 gene by RT-PCR at 50× dilution of the 0.025 mg/cc concentration (0.0005 mg/cc) (see Example 9). This concentration has also shown to optimize this gene to maximally produce the proteins that effect lipid synthesis that may be contributing to the abnormal lipids in MGD. Moreover, this concentration demonstrates a very good toxicity profile which is ideal and novel for chronic diseases which require long term constant exposure to the compromised ocular surface.

ELOV4 may be involved as an important and possible causative or disease related gene for the Meibomian Gland Disease in humans. Severe forms of MGD may show abnormal gene regulation or missense mutations in this gene.

All mammalian cell membranes are characterized by amphipathic lipid molecules that interact with proteins to confer structural and functional properties on the cell. The predominant lipid species are phospholipids, glycolipids, sphingolipids and cholesterol. These lipids contain fatty acids with variable hydrocarbon chain lengths between C14-C40, either saturated or unsaturated, that are derived from diet, synthesized de novo, or elongated from shorter chain fatty acids by fatty acid elongase enzymes. One member of the family of elongases, ELOngation of Very Long chain fatty acids-4 (ELOVL4), mediates the biosynthesis of both saturated and unsaturated very long chain fatty acids (VLC-FA; >C26) in the retina, meibomian gland, brain, skin, and testis.

Different mutations in ELOVL4 cause tissue-specific maculopathy and/or neuro-ichthyotic disorders. MGD mutations in ELOVL4 may cause a variable phenotypic disorder, and the inventors propose a possible mechanism, based on the role of fatty acids in membranes, which could explain the different variations and severity of the MGD phenotype. Adv Exp Med Biol. 2016; 854:129-35. doi: 10.1007/978-3-319-17121-0_18.

Regarding cell and molecular therapy, corneal, conjunctival or cells from the meibomian gland (i.e., meibocytes), stem cell therapy on patients may be very feasible knowing any gene abnormalities or missense mutations are critically important in understanding the pathogenesis of MGD and its subsequent treatments.

As reported, Karan et al. (2005) generated transgenic mice expressing the 5-bp deletion mutant form of human ELOVL4. They found that the mutant ELOVL4 exerted a dominant effect on the accumulation of undigested phagosomes and lipofuscin by the retinal pigment epithelium (RPE), which was followed by RPE atrophy. Subsequently, photoreceptor degeneration occurred in the central retina in a pattern closely resembling that of human Stargardt-like macular dystrophy and age-related macular degeneration. Karan et al. (2005) concluded that these ELOVL4 transgenic mice provide a good model for both STGD and dry ARMD.

In knock-in mice expressing the Elovl4 5-bp deletion mutation, Vasireddy et al. (2007) observed that heterozygous mice exhibited progressive photoreceptor degeneration, whereas homozygotes (del/del) displayed scaly, wrinkled skin, had severely compromised epidermal permeability barrier function, and died within a few hours after birth. Histopathologic evaluation of the homozygous pups revealed no apparent abnormality of vital internal organs; however, skin histology showed an abnormally compacted stratum corneum (SC), and electron microscopy revealed deficient epidermal lamellar body contents and lack of normal SC lamellar membranes.

Lipid analyses of epidermis from homozygous mice revealed a global decrease in very long chain fatty acids (VLCFAs) in both the ceramide/glucosylceramide and free fatty-acid fractions. In addition, Elovl4 del/del skin was devoid of the epidermal-unique omega-O-acylceramides, which are key hydrophobic components of the extracellular lamellar membranes in mammalian SC. Vasireddy et al. (2007) concluded that ELOVL4 is required for generating VLCFAs critical to epidermal barrier function, and that lack of epidermal omega-O-acylceramides is incompatible with survival in a dessicating environment.

MGD may be related to lipid production and homeostasis of unique hydrophobic components. Interestingly, Stgd3 mice, which harbor mutations in ELOVL4 that have been shown to decrease the levels of its biosynthetic lipid products, show changes that resemble clinical findings in patients with the evaporative type of dry eye disease, including increased eyelid blink rates, a reluctance to maintain their eyes fully open, protruding meibomian gland (MG) orifices, and anatomical changes within the MG (see Anne McMahon, Hua Lu, Igor A. Butovich; A Role for ELOVL4 in the Mouse Meibomian Gland and Sebocyte Cell Biology. Invest. Ophthalmol. Vis. Sci. 2014; 55(5):2832-2840. doi: 10.1167/iovs.13-13335). Thus, molecular approaches targeting ELOVL4 are expected to open new treatment approaches in humans with MGD.

This example provides a method for treating Meibomian Gland Disease or any ocular surface disease in a subject comprising obtaining a biological sample from the subject with Meibomian Gland Degeneration or any ocular surface disorder, determining the presence of a mutation in a nuclease hypersensitive region or a transcriptional regulatory region of a meibomian gland cell i.e. ductal cell or meibocyte, corneal cell, or conjunctival cell transcription factor gene, ELOVL4 gene, and administering an ocular surface meibocyte, ductal epithelial cell, corneal epithelial cell, or a conjunctival epithelial cell, a progenitor cell or a pluripotent stem cell lacking the mutation so as to form part or all of the diseased lid glands or/or ocular surface in the subject, thereby treating MGD and ocular surface disease in the subject.

According to this example, the biological sample includes tissues, cells, or nucleic acid from the subject. The nucleic acid can include DNA, RNA or a combination thereof. The mutation can be any mutation which results in altered expression of the ELOVL4 protein, including a point mutation; a substitution, insertion, deletion, transversion, or frameshift mutation; or a chromosomal duplication, inversion and translocation. According to this example, the mutation is present in the subject with MGD or ocular surface disease and is not found in a non-affected subject (i.e. without MGD or ocular surface disease). The mutation can occur in the nuclease hypersensitive region or transcriptional regulatory region of the ELOVL4 gene or genes, or can occur upstream of the ELOVL4 gene or within the coding region of the ELOVL4 gene.

Biopsy procedures for the eyelid are known (see Diagnostic Atlas of Common Eyelid Diseases by Jonathan J. Dutton, Gregg S. Gayre, Alan D. Proia. Taylor and Francis Group, LLC. 2007). These would be performed according to standard protocols used when taking a biopsy for pathology. Biopsy procedures for the eyelid include shave biopsies, incisional biopsies, excisional biopsies, map biopsies, and wedge biopsies. According to this example, a portion of the tissue sample would either be immediately put in culture or cryopreserved with the use of a cryoprotectant, such as DMSO or glycerol, for later culturing. Thus, the biopsy procedures need not be elaborated here.

The cells from the portion of the biopsy may be cultured through a variety of methods known for tissue culture or primary cell culture. For example, for primary cell culture, the tissue sample may be first dissected to remove fatty and necrotic cells. Then, the tissue sample may be subject to enzymatic or mechanical disaggregation. The dispersed cells may then be incubated, and the media changed to remove loose debris and unattached cells. Because primary cells are anchorage-dependent, adherent cells, they require a surface in order to grow properly in vitro. In one embodiment, the cells are cultured in two-dimensional (2D) cultures. Typically, a plastic uncoated vessel such as a flask or petri dish is used, and the cells are bathed in a complete cell culture media, composed of a basal medium supplemented with appropriate growth factors and cytokines. During establishment of primary cultures, it may be useful to include an antibiotic in the growth medium to inhibit contamination introduced from the host tissue. Various protocols for culturing primary cells are known and a variety of resources are available, including the ATCC® Primary Cell Culture Guide, available on the American Type Culture Collection (ATCC) website, Human Cell Culture Protocols (Methods in Molecular Biology), Mitry, Ragai R., and Hughes, Robin D. (Eds.), 2012.

According to this example, the cells of the biopsy are analyzed for mutations in the nuclease hypersensitive region or transcriptional regulatory region of the ELOVL4 gene. For example, genomic DNA isolated from a direct biopsy sample, cryopreserved biopsy sample, or a cultured cell sample can be subject to sequencing analysis to determine the presence of mutations. Various sequencing approaches are known, including Sanger (or dideoxy) method, Maxam-Gilbert, Primer Walking, and Shotgun Sequencing. Preferred are next-generation sequencing methods (also known as high-throughput sequencing), which include a number of different sequencing methods including Illumina (Solexa) sequencing, Roche 454 sequencing, Ion torrent: Proton/PGM sequencing, and SOLiD sequencing. Such next-generation techniques have been reviewed in the literature (see Grada and Weinbrecht, Next-Generation Sequencing: Methodology and Application Journal of Investigative Dermatology (2013) 133, e11; and Bahassi and Stambrook, Next-generation sequencing technologies: breaking the sound barrier of human genetics, Mutagenesis, 2014 September; 29(5):303-10). Next generation sequence methods may encompass whole genome, whole exome, and partial genome or exome sequencing methods. Whole exome sequencing covers the protein-coding regions of the genome, which represents just over 1% of the genome.

According to this example, healthy cells such as meibocytes, ductal epithelial cells, corneal epithelial cells, conjunctival epithelial cells, progenitor cells, or pluripotent stem cells lacking the mutation are cultured as donor cells for administration to a recipient (i.e. patient) with the mutation. In this example, the donor cells may be autologous, allogeneic, or xenogeneic. Autologous donor cells may be derived from the patient, and the mutation may be corrected in the patient's cells using gene editing technologies known in the art such as zinc finger nucleases, transcription activator-like effector nucleases (TALENs), meganucleases, and the CRISPR/Cas9 (see M. L. Maeder and C. A. Gersbach, “Genome-editing Technologies for Gene and Cell Therapy”, Molecular Therapy, 24(3):430-446, 2016). Allogeneic donor cells may be derived from a suitable human donor and cultured, such as a living donor or recently deceased organ donor, and the donor cells can be sequenced to confirm absence of ELOVL4 mutations. Xenogeneic cells may be derived from a suitable animal donor.

Alternatively, or in addition, such ELOVL4 mutations may be corrected in the patient through gene therapy techniques. Appropriate genomic editing vectors can be designed to replace the mutated DNA directly in the patient with “healthy” DNA. For example, a vector can be genetically engineered to include expression cassettes encoding Cas9 and a one or more guide RNAs. Such vector as well as a donor template such as a plasmid or oligonucleotide, can be administered to the ocular surface or eyelids of a patient. The sgRNA and donor template are designed to insert the non-mutated ELOVL4 promoter in place of the mutated ELOVL4 promoter through homology-directed repair.

Specific Aspects of this Example are enumerated as follows:

Aspect 1 is a method for treating Meibomian Gland Disease (MGD) or any ocular surface disease in a subject comprising:

a) obtaining a biological sample from the subject with Meibomian Gland Degeneration or any ocular surface disorder, such as macular degeneration, including progressive macular degeneration or where the macular degeneration is not a progressive macular degeneration, or Stargardt's disease;

b) determining presence of a mutation in a nuclease hypersensitive region or a transcriptional regulatory region of a meibomian gland cell, e.g., ductal cell or meibocyte, corneal cell, or conjunctival cell transcription factor gene, ELOVL4 gene, photoreceptor cell, e.g., rod photoreceptor cell, cone photoreceptor cell or photosensitive retinal ganglion cell, or retinal progenitor cell, such as a multipotent cell which can give rise to all cells of the retina, which retinal cell or progenitor cell can be derived from a pluripotent call or induced pluripotent cell; and

c) administering an ocular surface meibocyte, ductal epithelial cell, corneal epithelial cell, or a conjunctival epithelial cell a progenitor cell or a pluripotent stem cell lacking the mutation so as to form part or all of the diseased lid glands or/or ocular surface in the subject, thereby treating MGD and ocular surface disease in a subject.

Aspect 2 is a method of aspect 1, wherein the biological sample comprises a cell from said subject.

Aspect 3 is a method of any preceding aspect, wherein the biological sample comprises nucleic acid from said subject.

Aspect 4 is a method of any preceding aspect, wherein the nucleic acid comprises DNA, RNA or a combination thereof.

Aspect 5 is a method of any preceding aspect, wherein the mutation is a point mutation.

Aspect 6 is a method of any preceding aspect, wherein the point mutation is present in said subject and wherein the point mutation is not found in non-affected subject without MGD or ocular surface disease.

Aspect 7 is a method of any preceding aspect, wherein the point mutation occurs in the nuclease hypersensitive region or transcriptional regulatory region of the ELOVL4 gene or genes.

Aspect 8 is a method of any preceding aspect, wherein the point mutation occurs in the nuclease hypersensitive region or transcriptional regulatory region upstream of a retinal transcription factor gene, ELOVL4 gene.

Aspect 9 is a method of any preceding aspect, wherein the mutation is a transversion mutation.

Aspect 10 is a method of any preceding aspect, wherein the MGD is congenital or infantile.

Aspect 11 is a method of any preceding aspect, wherein the MGD is an ELOVL4 related gene abnormality.

Aspect 12 is a method of any preceding aspect, wherein the mutation or genetic change has a high penetrance, wherein about 70% of individuals with the mutation or genetic change on an average develop MGD or ocular surface disease.

Aspect 13 is a method of any preceding aspect, wherein the mutation or genetic change is inherited through germline and not a somatic mutation.

Elongation of Very Long Chain Fatty Acids-Like 4; ELOVL4

HGNC Approved Gene Symbol: ELOVL4

Cytogenetic location: 6q14.1 Genomic coordinates (GRCh38): 6:79,914,811-79,947,597 (from NCBI)

Source:

Online Mendelian Inheritance in Man, OMIM (TM). Johns Hopkins University, Baltimore, Md. MIM

Number: 605512: 01/02/2018: Retrieved from http://omim.org/entry/605512

(incorporated by reference in its entirety)

The present application incorporates the following journal articles by reference in their entireties:

Agbaga, M.-P., Brush, R. S., Mandal, M. N. A., Henry, K., Elliott, M. H., Anderson, R. E. Role of Stargardt-3 macular dystrophy protein (ELOVL4) in the biosynthesis of very long chain fatty acids. Proc. Nat. Acad. Sci. 105: 12843-12848, 2008.

Aldahmesh, M. A., Mohamed, J. Y., Alkuraya, H. S., Verma, I. C., Puri, R. D., Alaiya, A. A., Rizzo, W. B., Alkuraya, F. S. Recessive mutations in ELOVL4 cause ichthyosis, intellectual disability, and spastic quadriplegia. Am. J. Hum. Genet. 89: 745-750, 2011.

Ambasudhan, R., Wang, X., Jablonski, M. M., Thompson, D. A., Lagali, P. S., Wong, P. W., Sieving, P. A., Ayyagari, R. Atrophic macular degeneration mutations in ELOVL4 result in the intracellular misrouting of the protein. Genomics 83: 615-625, 2004.

Bernstein, P. S., Tammur, J., Singh, N., Hutchinson, A., Dixon, M., Pappas, C. M., Zabriskie, N. A., Zhang, K., Petrukhin, K., Leppert, M., Allikmets, R. Diverse macular dystrophy phenotype caused by a novel complex mutation in the ELOVL4 gene. Invest. Ophthal. Vis. Sci. 42: 3331-3336, 2001.

Cadieux-Dion, M., Turcotte-Gauthier, M., Noreau, A., Martin, C., Meloche, C., Gravel, M., Drouin, C. A., Rouleau, G. A., Nguyen, D. K., Cossette, P. Expanding the clinical phenotype associated with ELOVL4 mutation: study of a large French-Canadian family with autosomal dominant spinocerebellar ataxia and erythrokeratodermia. JAMA Neurol. 71: 470-475, 2014.

Conley, Y. P., Jakobsdottir, J., Mah, T., Weeks, D. E., Klein, R., Kuller, L., Ferrell, R. E., Gorin, M. B. CFH, ELOVL4, PLEKHA1 and LOC387715 genes and susceptibility to age-related maculopathy: AREDS and CHS cohorts and meta-analyses. Hum. Molec. Genet. 15: 3206-3218, 2006.

Edwards, A. O., Miedziak, A., Vrabec, T., Verhoeven, J., Acott, T. S., Weleber, R. G., Donoso, L. A. Autosomal dominant Stargardt-like macular dystrophy: I. Clinical characterization, longitudinal follow-up, and evidence for a common ancestry in families linked to chromosome 6q14. Am. J. Ophthal. 127: 426-435, 1999.

Giroux, J.-M., Barbeau, A. Erythrokeratodermia with ataxia. Arch. Derm. 106: 183-188, 1972.

Griesinger, I. B., Sieving, P. A., Ayyagari, R. Autosomal dominant macular atrophy at 6q14 excludes CORD7 and MCDR1/PBCRA loci. Invest. Ophthal. Vis. Sci. 41: 248-255, 2000.

Karan, G., Lillo, C., Yang, Z., Cameron, D. J., Locke, K. G., Zhao, Y., Thirumalaichary, S., Li, C., Birch, D. G., Vollmer-Snarr, H. R., Williams, D. S., Zhang, K. Lipofuscin accumulation, abnormal electrophysiology, and photoreceptor degeneration in mutant ELOVL4 transgenic mice: a model for macular degeneration. Proc. Nat. Acad. Sci. 102: 4164-4169, 2005.

Lagali, P. S., MacDonald, I. M., Griesinger, I. B., Chambers, M. L., Ayyagari, R., Wong, P. W. Autosomal dominant Stargardt-like macular dystrophy segregating in a large Canadian family. Canad. J. Ophthal. 35: 315-324, 2000.

Maugeri, A., Meire, F., Hoyng, C. B., Vink, C., Van Regemorter, N., Karan, G., Yang, Z., Cremers, F. P. M., Zhang, K. A novel mutation in the ELOVL4 gene causes autosomal dominant Stargardt-like macular dystrophy. Invest. Ophthal. Vis. Sci. 45: 4263-4267, 2004.

Ozaki, K., Doi, H., Mitsui, J., Sato, N., likuni, Y., Majima, T., Yamane, K., Irioka, T., Ishiura, H., Doi, K., Morishita, S., Higashi, M., and 11 others. A novel mutation in ELOVL4 leading to spinocerebellar ataxia (SCA) with the hot cross bun sign but lacking erythrokeratodermia: a broadened spectrum of SCA34. JAMA Neurol. 72: 797-805, 2015.

Vasireddy, V., Uchida, Y., Salem, N., Jr., Kim, S. Y., Mandal, M. N. A., Reddy, G. B., Bodepudi, R., Alderson, N. L., Brown, J. C., Hama, H., Dlugosz, A., Elias, P. M., Holleran, W. M., Ayyagari, R. Loss of functional ELOVL4 depletes very long-chain fatty acids (greater than C28) and the unique omega-O-acylceramides in skin leading to neonatal death. Hum. Molec. Genet. 16: 471-482, 2007.

Zhang, K., Kniazeva, M., Han, M., Li, W., Yu, Z., Yang, Z., Li, Y., Metzker, M. L., Allikmets, R., Zack, D. J., Kakuk, L. E., Lagali, P. S., Wong, P. W., MacDonald, I. M., Sieving, P. A., Figueroa, D. J., Austin, C. P., Gould, R. J., Ayyagari, R., Petrukhin, K. A 5-bp deletion in ELOVL4 is associated with two related forms of autosomal dominant macular dystrophy. Nature Genet. 27: 89-93, 2001.

It will be understood that the Specification and Examples are illustrative of the present embodiments and that other embodiments within the spirit and scope of the claimed embodiments will suggest themselves to those skilled in the art. Although this disclosure has been described in connection with specific forms and embodiments thereof, it would be appreciated that various modifications other than those discussed above may be resorted to without departing from the spirit or scope of the embodiments as defined in the appended claims. For example, equivalents may be substituted for those specifically described, and in certain cases, particular applications of steps may be reversed or interposed all without departing from the spirit or scope for the disclosed embodiments as described in the appended claims. Additionally, one skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features.

It is noted in particular that where a range of values is provided in this specification, each value between the upper and lower limits of that range is also specifically disclosed. The upper and lower limits of these smaller ranges may independently be included or excluded in the range as well. Further, all of the references cited in this disclosure are each individually incorporated by reference herein in their entireties and as such are intended to provide an efficient way of supplementing the enabling disclosure of this invention as well as provide background detailing the level of ordinary skill in the art.

1-13. (canceled)

14. A method for reducing or preventing one or more signs, symptoms, causes or effects of dry eye or meibomian gland dysfunction comprising:
administering to an ocular region of a subject a composition comprising at least one aldosterone antagonist, or isomer, salt, or solvate thereof and a pharmaceutically acceptable carrier for reducing or preventing one or more signs, symptoms, causes or effects of dry eye or meibomian gland dysfunction.

15. The method of claim 14, wherein the at least one aldosterone antagonist, isomer, salt, or solvate thereof is selected from one or more compounds of Formula (I): 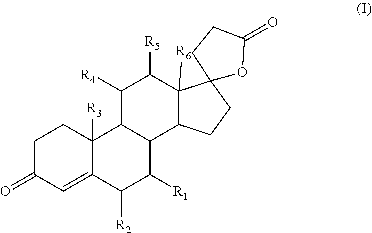
wherein R₁, R₂, R₃, R₄, R₅, and R₆ may each independently represent a hydrogen atom, an oxygen atom, a fluorine, chlorine, bromine, or iodine atom, a saturated or unsaturated, branched or unbranched, substituted or unsubstituted aliphatic or aromatic hydrocarbon containing between 1 and 20 carbon atoms, such as an alkyl group, an alkenyl group, an alkynyl group, an alkoxy group, an acyl group, an acetyl group, an aryl group, an aryloxy group, an acrylyl group, a carbonyl group, a cycloalkyl group, a hydroxyalkyl group, an alkoxyalkyl group, a hydroxycarbonyl group, an alkoxycarbonyl group, an acyloxyalkyl group, a heteroaryl group, a heterocyclyl group, a ketal group, an acetal group, an amine group, an amide group, an imide group, an azide group, a sulfur-containing group, a thiol group, a sulfide group, a disulfide group, a sulfinyl group, a sulfonyl group, an acetylthio group, a formyl group, a furyl group, a hydroxyl group, a hetero atom, a cyano group, or an ester, ether, ketone, or aldehyde functional group, as well as substituted groups thereof; and
when R₁ and R₂ are each a hydrogen atom, there is a C—C double bond present between the carbon atoms to which R₁ and R₂ are attached.

16. The method of claim 15, wherein the aldosterone antagonist is spironolactone.

17. The method of claim 14, wherein the pharmaceutically acceptable carrier is a solution, a suspension, or an emulsion and the pharmaceutically acceptable carrier is chosen from water, an aqueous solution, a polymer or a nonionic surfactant.

18. The method of claim 14, wherein the total amount of aldosterone antagonists is between 0.0005% to 1%, based on weight or volume of the composition, or between 0.005 mg/mL to 10 mg/mL.

19. The method of claim 14, wherein the composition further comprises one or more antibiotics, steroids, anti-inflammation agents, analgesics, surfactants, chelating agents, buffering agents, pH adjusting agents, adjuvants, protein-based materials, and combinations thereof.

20. The method of claim 14, wherein the one or more signs, symptoms, causes or effects chosen from impaired vision, burning sensation, redness, irritation, grittiness, filminess, inflammation, discomfort, pain, chemosis, chalasis, engorged vasculature, anterior lid margin vascularization, zone A posterior lid margin vascularization, eyelid disorders, swelling, lipids, vital staining, Schirmer's score, or meibomian gland obstruction, secretion, viscosity, secretion turbidity, loss, drop out, or dysfunction.

21. The method of claim 14, wherein the composition is a liquid and is administered as an ophthalmic drop to the ocular region of a subject.

22. A method for treating meibomian gland dysfunction comprising:
administering to an ocular region of a subject a composition comprising an effective amount of at least one aldosterone antagonist selected from spironolactone, eplerenone, canrenone, prorenone, mexrenone, or an isomer, salt, or solvate thereof of, for treating meibomian gland dysfunction, and a pharmaceutically acceptable carrier.

23. The method of claim 22, wherein the at least one aldosterone antagonist selected from spironolactone, eplerenone, canrenone, prorenone, mexrenone, or isomer, salt, or solvate thereof, is selected from one or more compounds of Formula (I): 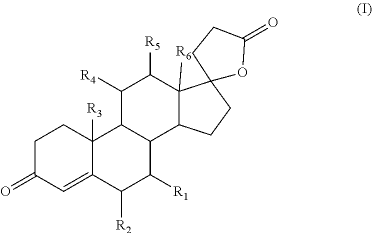
wherein R₁, R₂, R₃, R₄, R₅, and R₆ may each independently represent a hydrogen atom, an oxygen atom, a fluorine, chlorine, bromine, or iodine atom, a saturated or unsaturated, branched or unbranched, substituted or unsubstituted aliphatic or aromatic hydrocarbon containing between 1 and 20 carbon atoms, such as an alkyl group, an alkenyl group, an alkynyl group, an alkoxy group, an acyl group, an acetyl group, an aryl group, an aryloxy group, an acrylyl group, a carbonyl group, a cycloalkyl group, a hydroxyalkyl group, an alkoxyalkyl group, a hydroxycarbonyl group, an alkoxycarbonyl group, an acyloxyalkyl group, a heteroaryl group, a heterocyclyl group, a ketal group, an acetal group, an amine group, an amide group, an imide group, an azide group, a sulfur-containing group, a thiol group, a sulfide group, a disulfide group, a sulfinyl group, a sulfonyl group, an acetylthio group, a formyl group, a furyl group, a hydroxyl group, a hetero atom, a cyano group, or an ester, ether, ketone, or aldehyde functional group, as well as substituted groups thereof; and
when R₁ and R₂ are each a hydrogen atom, there is a C—C double bond present between the carbon atoms to which R₁ and R₂ are attached.

24. The method of claim 22, wherein the aldosterone antagonist is spironolactone.

25. The method of claim 22, wherein the pharmaceutically acceptable carrier is a solution, a suspension, or an emulsion and the pharmaceutically acceptable carrier is chosen from water, an aqueous solution, a polymer or a nonionic surfactant.

26. The method of claim 22, wherein the total amount of aldosterone antagonists is between 0.0005% to 1%, based on weight or volume of the composition, or between 0.005 mg/mL to 10 mg/mL.

27. A composition comprising from 0.0005% to 1%, based on weight or volume of the composition, or between 0.005 mg/mL to 10 mg/mL of one or more aldosterone antagonist, or isomer, salt, or solvate thereof, in suspension, solution or emulsion in water, an aqueous solution, a polymer or a nonionic surfactant as a carrier.

28. The composition of claim 27, wherein the one or more aldosterone antagonist, or isomer, salt, or solvate thereof is chosen from one or more compounds of Formula (I): 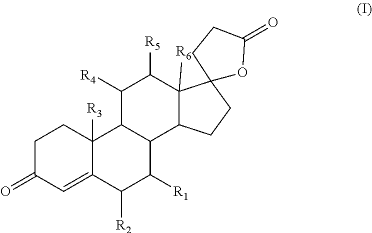
wherein R₁, R₂, R₃, R₄, R₅, and R₆ may each independently represent a hydrogen atom, an oxygen atom, a fluorine, chlorine, bromine, or iodine atom, a saturated or unsaturated, branched or unbranched, substituted or unsubstituted aliphatic or aromatic hydrocarbon containing between 1 and 20 carbon atoms, such as an alkyl group, an alkenyl group, an alkynyl group, an alkoxy group, an acyl group, an acetyl group, an aryl group, an aryloxy group, an acrylyl group, a carbonyl group, a cycloalkyl group, a hydroxyalkyl group, an alkoxyalkyl group, a hydroxycarbonyl group, an alkoxycarbonyl group, an acyloxyalkyl group, a heteroaryl group, a heterocyclyl group, a ketal group, an acetal group, an amine group, an amide group, an imide group, an azide group, a sulfur-containing group, a thiol group, a sulfide group, a disulfide group, a sulfinyl group, a sulfonyl group, an acetylthio group, a formyl group, a furyl group, a hydroxyl group, a hetero atom, a cyano group, or an ester, ether, ketone, or aldehyde functional group, as well as substituted groups thereof; and
when R₁ and R₂ are each a hydrogen atom, there is a C—C double bond present between the carbon atoms to which R₁ and R₂ are attached.

29. The composition of claim 27, wherein the aldosterone antagonist is spironolactone.


SEQUENCE LISTING (3 sequences)

SEQ ID NO: 1 - DNA, 39787 bp, Homo sapiens
gtgtatagtgtgtatggattcttcaggttcaagacagtttctccaagatcagaggggggtcatttatctttgtatggttcacaggcaggtgagtcctactgcaaagttgctgagtccataaaattatggttcatttactggaaagtttttaagcatttattcacatataaccattttccttaagaagcattcctgggtcatcgggatggctggtaggagatagcttctggaatcaactaaatgcttgcctagaaccaagtatttttctgatgtcttgggtttttaaaagtaagtccttgaaatacagctggtttctttcctctatcatggtgagtttatttcccctcaggctctttttatcttctcctaccctgaaaccagattgctcttcagtaataggaatatgagagcagggtgaaaaggcattccccattaacacttggaatttattaccagatttcttggaccaaagtttgttgatatccatgaagacagatatgttagagtagtaagtgaaggaaatcacaactcagacaatactcaattaagagtgacaaaacttttcttggtatagccaagccaggaaaaacgggcagaaccaccatggtttggccctcttatcactatatgcttgaatgtatgactaaaagtgtgggatatggccttgaaacatggctatgtgaagagagactgggctaatggctttgaccggtaaagaatacagattgggtgtacatattgggtcacaccaaaacaatgaatatccaatgtaagtaatttgtagttgtcattcttaggaaaggagtagcatatgtaccttagaaatatgcatcaggggttgcaaagctgagaaggcattctgatatatgattctggtggaggtggtggcgggtgaacctaacttattcccagcattcaatatgcctagttgaaaatcattataataaatttagtaagagacaaatagcaaactgggaaaatatatttgcaacatatgcagacaaactgttaataactttattataagaagatgtcttataaatcaataagaaaaagtagacacaactaatggaaaatgtctaaagaattcaaacaggcaattcctgaaagaaggctaaaattgcaataaacatgataatctgtcaagcaacaatgaaatatcaatctttacctctcagactggtaatgattacaaagcatacattcagtaaagagaatggggtggggaaatgagcaccttttcataaattttggtggaattgtgaattggtacatttctggaggttaagtgggcacgatgtgtcaaaatttgtaaacttaaaaccctttgaccaggtacagtggctcacacctgtactcccagcactttgggaggccaaggcaggcagatcacatgacaccaggagtttgagactagcctacctaacatggtgaaaccctgtctctaataaaaatacaaaaattagctgggtgtgatggcacacacccgtaatcccacctactcgggaggctggggcatgagaatcccttgaacccgagaggcagaggttgcagtgagccgagactgggccactgcactccagcttgggcgagacagtgggatgccgtctcaaaacaaaaacaaccacctctccacccctaaaaaaagtcccaaacctttgacccagaaatttcccttgtaaatatttatgctaagagttattaaacaagtgcacaaaattgcatattgtggatactaatagcccaattgatacagtagttcaaatttgaaacaatctaaatgctaatctatagggtaaatggctacataaatgagtgtgtatccatataatggaatattaagtacttggtattacgttgaaaagtattcctgatgtattaaatttttaaaaaagcagattatagagtagcattattttaaaaaaccatctgtgttaagaaaaaaaggcttatataggcatgtaaaatttatttagaatgggagacaaaattataggggggattttcattttgtttctttttttgttattttatctgtgctaatgtattatcaggataatctatgtccatttaaataaaataaaaataattcatacaagctaacaaaaaggtaaacatctttaaaagtttgaatgaaacatatctcgtggaaaacattgtaattccaagttagtcaagcacctgtgttatatgatggctataacgtgggggaggagtaatgagcagaataaaacaaaagactggaactaatagtagttagtgttatcaacaattttctccattttctctaagtagcatccatgtagcaaaatttacttctagaagattcaaagtctttctctacaactctttaattaggttatttctttttactcctgactcaacaggcttccaggttcacttttctttgaaagcagaggttgctgacaatattaccaacatggaaaggtggactaggaaaaaaaagatggatagcttgatttatgttgctaaccttgtatggactgtaggacatttaaaaatattttgtactgggtgtggtggctcactcctgtaatcccagtggttttgaaggccaaggcaggaggattgcttgcaggcaggaatttgagaccagccggggcaacacagccagtcctccttctctatcagctatttaggaggctgaggcaggaggatgaggcaggaggatagctcgagcccaggaattcgatgttgcactaacctatgattacaccactgcaatccagcctagctgatagagtgagaccctgtttctaaaaagaaaaaaatattgctattttatacaaaaaaatttatgataaaatgtcctctcctttaggttttgtaggtaactgtatcctatttgcatttgatccacatgaaagaactctgttctcaaagtttgcagatgctgttgttgcttgtatttgcttttactctgttatgatgctaccaagattttggttagatggctgttattctaggtggcagacatgcgggtttccctccctttgcacaactaaacaaggtaggctttggttctgggcagtcactggtatttacaaaccacttattcatgccaggtaatagggttccagattgggtaagttttatgttctggttcttggtatatgtttagacagcttataaatgtaaataaacattctactcttacaggtggagtgtggacacggtgatatccgtgtgaccgccattcccagctgaaggttatctcatctatgattttcttatacgtgactggcaccccctagtgaatagggacttaagttgacctccacggtagaagaaataccagggaaatacctgaagctgttttaacaatttctccttgtattaagtattatgctgcagttttgcgtgtgtgaatggaagtatgggtagagatctgttctccctaaaaactccaggattccacaatatagaaatagtaatcaaatttttaggtgaagctcgaactaatccgaactttgttagatcatcactgtaaatgaatgggtatttatccactccctaaatgaagagacttgactggatttcttttttttatatagctactagaatctgttacacataatttaggattgagacttgagaaattgtcattccaatccagaaaactttagatttgcaaatatatttgacaaattaataaattaacattttatttggttaatttcaagaatagggcatttaaagaagtctgtgtttgctttagttcggcaataaagttcctgccactcacaataatccttattattctctgaaagacatgttatatttttgtcatcataaatatttattaattactgtttatagcactgggttaggtactcatcaagcaaccaaaaataattcttaccatctaggatgcttccaatataaaatatagacaatatataaccaggtcaattgggaaatagatcatttcagtatgataaaagatagtattcacattaacagtgtgaaagggcaggaacaataagacacttgactcactggtctttaaaatgtagcatccaaaatgagcaagtggagaaaaggttaaacaagtaggtgacacatttaaaaaacaagtagatgaaaggactattctcaaaaatcttgttttatgtgagaaaccatcaaattatgaattccaagtactgtattttttttacttttcaagggtaggctctcctataccttatctaaacaatttttcaaaatagccacaattactttgttttcctctctacactaaattgccctttgcctcttgagcgattatctttttcagattcacctcaacttcttcaggttcaagcggacttcacctgtaagcccctctcggttctccctcttctctgaactactaatggcctaatttagcacaattatattgctttgttcattccatgtatagtaaaagagtctacaaaacacatgcaagcattcatgcaattatatgttgatttgttcatgggtcgaccccaaagtctattctccatcgctgaagcatggaagacaaatacccttcacttcttcagaggcataacacatgcacttctcttgtcatggtgacaggcatgtgctggtggaggtcaaagaaacaggaacacaagtgaaatcgaggtgagtgtcaggtaaggaccaaagcaccacgcctacctcatctttgcccacagaacacccattcttcccgtgtcctgtttcccaggacgtatccggggcggataagaaatcacccgtggggaggcggtgaactcctccgcaggggccgatgcccgggacaggggcggggaaggctaatgaggcgacttgtgcggggaggggccaaggaggagcccaggtgtcccgctcccgctcgacggcgcgcgcctgcgcgagcccagttggcgtcgcacccttgagcgcagcatccctacgccagcgagtcccaatactagggagggagggagggaggaggggcggccggccccccgcccccgcgcgcggccacgtgacgccggctgaggagattggaggggcggctgcgcgaggctgcagactggtgcagcgcactgtgctggcggctgggcctcctccacctcctcgtctttctcccgggaaccttgacgacgccttccgcttggccctgccttctgccgcatccccgccgccgcggcgccttgaggagcaggagaagacgcagccgggccgccgccgttagaggggttcccggccgccgctcgccccgtcggccgccaccgcctccggggtcagccctctctctgggtctccgctttctcctgccgccagcgcccgctcatcgccgcgatggggctcctggactcggagccgggtagtgtcctaaacgtagtgtccacggcactcaacgacacggtagagttctaccgctggacctggtccatcgcaggtaaagccgctgacttccccatcctcgctcggtcccccgcggggggtcaccggcccctggtctcgcagctcccgggcccggccccacaggcccccgcgccctgcggctttcggatgctgcggatcagcggaggggccggcgcgggcactgcggttgagcactggttaatctgatccgaatgttaaggcctcccctcccccgacactcgcagggttttcttctgagctccctccatctctcatcagctctcggatgcgggagctgacatatcagatggagcctgtgcagccaggagctcctcgcaggagccgccccaccctgtgctcccacatctcccgagctcgcctccctcttccccgtgcacaggattccttttaacatttagtcctattttgtatggagagcgggctggggaggaaggagggcttcaggggcagctgaaggtacaggaggtttcttcagcctcctggtcgctttgacagaaccctgctaatcttttcttctatctagctccttcatctatagtataattcggactgaaaagtaaaataatattgtttggctccttgaaccttgaagaatgttagtttccgaggcattttcaacagcaaatatgtttgaagtgattgtctataatggctttgcgcactgcagtgcagtcagggggagggggaggccaaaacaccggggaagaaatcagtctttaggaagggggtctctacagcaacacctagaggttgtcccagatgcctacatatatacagttcttaaatcagcaaacttttctttagctgttgcagttaatttggcagggctgtcaattgtttgatttatgcagctttcctagcgtccaactgtgatattatatttttacattgtactgtttatttcttcatttgcttcgctgcttttgattttaaagcatttaaaagtctttgaaatctaaaataaggtttaaaggatttcagaaatgattttacgaggtcaatttttctggtccttttgcagtatcacactgcatcctatcagtgaaggatattaacgttccatcttatgactctgccttccctacttcgaaatgaaaatgtactcctgtagtttttgtgttcattgttgttagaggaaattagacagtggttacagcatgaatgtagtccgatacatagggcttatgttttgggaaaaacgtatgctggaattatttttgtgagagacaaaatgattatttggcattaatactgcattcacttctttagaaaagtatgttctttttcagaagatctcaactttgacactgggaaataagctatttaataagccattacagctgaaaatggttaaaaggcagttttcaatggagtgtttcttcagtttttttttttgtttctatgcgctattacactgatgttacactgtacctgcaaaaactgggaaagatgtgcccttcccttcacgtctgtgatttcggttctttaaacgttgcatcagggcttcccattacacaattgtatagagctgaacatcagacaagttctttagcaggtaattgcacttcgcacacctaggagtcagacactgttgagatactccagtctccatatagccagagccacacccaggagttctcggtcctgtcattcctatcctatcccggagctttcccatctcttgagtctcagaaggaaaaattgagggcaaaggtagaatacacctttctgtgatggtgaattccttcctgatgatgtttgtgtaaaagaaaccacacacacacaaatgaaacaggaaattttgtgcagttgccgcatgtgaaagcttgctactaggcttcgaagggcatataaaataatataatattgtgggccaagcctgagaagtgagagtagccttagaacacaagtgacctctcactagcaggccgaaggaaggtatttgcctatgctttgactatccacttacgaaacctgagactgcttccttgctttctgtaatgagctaccttatgtaggcagatggtatttcttaaccaacagagaggcttagtggatatagtctgttctgctgtactgctgtgtgaggaggagtatttttcaggtccaggccgaatagacctgaagtctgtggtgtcccatttacgtttactctccctcattctatgcccctcgtgttccttttttttttttttttttttttttggactcattcactctgctctgattccacctctcagcataatttgatgaaaatctgggtgggggatatcaaaatagaaaagtagcttataagatgaggttaaacaaaagttggtgagtcaaatgtttggaaagaaggggaaacagaaatgggcgggatagaaagatggtcttgagaaatagtgatgagtgtggtgaaaggatgcaaattattgagggagagcaagaaatgaggtaggctccttgtccttggctgctctcattcctagtgctgtagttacatgcttctttcattcttttattaattcattaaatgccattttgtgctattatatcaaatggtgctttaaatatccaaacatatccaaagcaatgtctgaaaactgggcttttcagaggcacatatgtagcaaatacaaaatatggtccataataatgttaacaacaaaaatgttacttttactacagtattaacactttgtacattggaattgaaagtttgaaagagctatacttttagaaaaacaacaaaaataatttttatttctctgtatttgtagacattttgaacgaggaaattagcgttggtttatgttaaagtataggctggtgtaggactttatactagttgttagtattagagtaggtattccatgtcatcatgttagtttttttcttctattctttcacttcatgagtacatttaaattattaggattggagtcctaggctcaacgtatacatctaataaaagttgtatatacattttttaaactgatgaatctaaatattccccaatctatgaatacccaacttatctacattattcatttattcatgcttttaataaatgtttaatagcttttttgcatacctgacatttagcatataaacaccctagagagttagtaggatgcttacggtaaagccaggtgtctcctgtgaggccatgtatcttctgaccatttgtttctaaatagctgttgttcacagggtatgggtgctgtctgttggttgggggtggtaatgattggcattggccctcttttcgcagatatagtaccctgcattttggtgaaaaaaattgtctacttggtagttgcagttagaacataacttgctgggtccttgaattaactttggaggggtcagaaagaagggatgagtggtacaggaagagggaggagagctgtaggatgacgaaatgtggaagccaggcttatgataccatttctttccgtatttacttgccatgtacaggcacagttctaggctctggaaaggaaaggaggacacttggtcactgccctcatgcatcttgcaaagtctagggggtcactgtcattcccctttccttgatatcatggtctggaggcagtgtgatctgtcagactcaggactagctggggcctagggaagatcagaaaagtccttccctgctcttctcatctctacttctcctaagtgaaaggaatatacagttgtttttagaatattttagctgtttcaccacagtttacttccctctacccttgaatgaggagttgatgtgagtactgtcagagtatttggagaggcatactagggcaaaaggtgtcaaaggcaaggggaacagcttgactgaaaaccatggaaactgggaaggtgcttaagggtcaggccagtgagtcatccaggtttgatgaagctgggggctagtgggaggggatgtttaactcagctcaaataagctgaagtaaaagcaggtttgtcttagggattgctaggtcccagaggagcatgtattttaagtattgtacagtatgcttacattaattttattattcgtcacagttaatgtttagtttttcatctatgttattatctaaaataggtttatttgtttggctaagcctggtaatgcagttttctttgaatgtttctttgagaaaagacattctgattccaagttgtaggcagctaattaaaaaaaaaaaagaaagaaagaaacatcaccagctcctaacatgggatctcccttggtcactgttttcagtgacgtcctaattatcctttactcataagctccttgaagcctctgggatgtccatgccattttatggttttttgttttggtttggtttttattgtcttttccttcaccacagtttgatttttggtggtagactgctagactttaatagctaatgctcttatccattccccaatgatagcataaagtaaaacatcctattgcagatggaattccaggagggtaaacaatgcaggttctaaaacgggtgtcaggacacagttttaactattttaacttagattctttggcttaggaagacaaagttttaagaattatgcagataattatatcagatggcacttttgtaaacttaagagccacagagcacttggtcatcctttaaaaattcaatttgttttcttatttttttgctcaagtcctgtggataatgtcacctgtaaatgctttaacattattaacgtaagtattttcatttgttaagccacttattattctgaattaggcatatattagcacaccgaaatgagtagtgtaacagtctatcttttttcactttagatgtttttttcgtcttcattatcaggacattttgcctgttaacaaacgaaggtactattgttttattttctattagaatatatattttaaaatcatgtgggtttggatttcagaaaggagatgggaggaaacacatggccttttaaagccaaggtccacattaactttctcgcttgatccttcagtattcccctgcccaaagacctgcagctctagccaactgggataactcactgacccttaagtaaaatgtgtacatgtccacctttatgtttctcatcccaccattccccttgcctttacccaccttttccctgtttacctctccagataccttttaccagaacccagtacaatagtcctcctttgcaaagcctttcttgtttatggtaccctggagcaagttgttgtctcacaggagctcattgtctttagtaattactcacgtatttcctgtgccattccttctatgaccctagtgtcaaactgtttaaactttcgttattatttaccacttcactcctttgtatctttctctccaacatcagagattatcacatatatttgttgatatgatatgtatcaggagatgaccctcttaatctagcataattttgaatcatgatatattgtggttgctaaacatgaataaggctttagaccacactaaattacagaaggtttttgtttttgtttttcatagagtggggatttcaccatgttgccctggctgctcttaaactcctggtgtcatgtgatctgcccacctcagccccacaaagtgctgggattacaggcacgacccactgtgcccggccagagagttctttaaaagaatgatattttttatgagttggtcattaattcagttgccgttcactaaatttaatgagtaaatcaggccattaagtcatttagaagcacaaaacagataaaaaatgaagattccaagacatataaaatcagatatgattgttctcttcattgtctgtgcagaagcattgccttgtgtggaagtgaggagggcagtgtggtatagtgaatgggaagaattctggactgggatttagatgacctaggttttagtcctggtctgctgctgatttgctatgagagtttggacaagttcctttgccttcccaattctcagcattgttctctgtaaattaagagggttaaattaggtaatctgtaaggtcctacctgactctaaaagtctagatatagatatcactttctttattcttggaataggtaaagcaatttcaaataaatatacatttatcagggattcagaaacctcattctttatagtaaataaaatttttttccagcaaaactgttatcaacactgcctaaatagtacaaataacagattcttatttagttctttttactttttaaaatttgctttataagaaataggtagaggaatagagcaacaaaagtatacagatatttaagcgtctgttttgaaataacattgcttttgttgctgtaacactatacctgtttaattatctggattccttgaataaccaagagccaactattttatcgaaaacagtagcaataatattaaagaataaaattatctcaattatatattacatttattttgtgatactcagcttttgaggatggcatattatgtcgttttaggcaagttattcatgactctttgggtctcttatttctacctgtgctgtgagagggttgaactacataacctttaaggcttagtttattgaatccttataacatctcctaaaacagcagcctagatatcagtctaaactcagtcttcatatttttccaatagaaaatgtgaaattttaacatacatcattacaaatgattttctattataaacactttatatatgagcattttttacttaagtttggaagtatggcttaagatttttgtaaaattgataaaaatgtgtaggtcttacagaagagaaaacaaaagaaactaaatggttttggtataagtatgttccttaactaatcaaaatacaaatttttaaaatcttttgccatatatttttcttcatgaaatattaccatttgtaaacaaataccttagaatgatgttttatttatgattatattgtattagagtaaaatttttggaatttgaaagacaaatgcattagtccactgaggaaatcagttactcactgtcaggtataatcacataaaagcatttgtgattttgaactgcagaattccaagaaacaagcttttctgttttcatttttattcacttatcaaacaaaaacttgagcacagatgtgtgtttttaactaattttatatattttttcacacacgtacatagcagcattattcacagtagttaagaggtggaaacgacccagatgtccagtgttggataaatggataaacgcaacgtggtatatacatacaatggaatattattcagtcttaaaaaggaaagaaattctgacatgtgctacaacatagatgaaccttgttactaagtgaaatagtctagtcacaaaaagataattatgtatgtggcattatggttccacttatatggaccctctactgtagtcaaattcgggctgggcccagtggctcatgcctataatgctagcactttgggaggtggaggctcaaggatcacttgagcccaggagttcaagaccaacatgagcaacaaaaaaatattaaaaattagccaggcctggtgccatatgcctctagtctcagctattgagaaggctgagggagaaggatcacttgaacctgggaggtgaaggcttcagtgaaccataattgtgccactgcactccagcctgggtgacagagtaagatcctggcccaataaataaataaataaataaataaataaataaataaagtagtcaaattaatagaaacagaaagtagaaagatgtttgctagggcctggggagaaggagaactgatgacttgtttaataggtatagagtttcagtttttcaagatagaaaagttccagagagtcacaacaatgtggatatgcttaatgatactggactgtacacttaaaaatgattaagatggtatattttatgttatgtgcttttctttttaccgcaatgcaagaatttcatatattataaaattaatacattagcacctgtagaaatgatctctttagtgctcaatggtaaaacagtcaattcttctttattttttcaatttaaagtaaaatgactttataaagagactatatagctgttggcttaaaaaaaatccgagtggctagaaagtagcaaataacaagaaagaaaactttagctttgtattttgctttatggttttacaaacaacttcacaataactctgggagataggccttgaaggcattttttccatatatttagaagtctttgagatttacagaagttacgtaacttttgtaggtggtcactatagtgcaaatgaatgtgcatgggttgttttgtccctagcccagtgctcttgtcttcccgtcacactagccaagcaagaaagaaataatcaggcctgtaatctgtgtttctcttttattttaccttttcttttattgttatctacagagtatcactgtttagaatactaaatctgaacaaagtcataatattaccaaagatcatctaaagaaagacatctaaatattcaaaggtgctataaaggctggtgtatgagattaaactacaaagtgatatttattcatatactatatacatacacaaatatgtgaatatatgtagaatacaaaatgcgatatttagcctgagaagatggttaacttccatatgacggtgtaggattgtttaaaaaaagtgtattcaaagtgaattatgaaagtatttgccaaattgcataatacaagagctagaaacttgttagtgaaaggataatgaaattcattcaggactaaccgtattttacccagtggctattcaacttatagaactgttagagtcaagggaagattttggctaaagttcccattaactcacacactcacacactctaatggtatcttccatcttttttgctaaagggaaacaccatttcaattttcttaaagctttgagttcattggcagcattgggtggatggttgaacttggaactcccagattaagttttatgactactttttctgagaagcccagcaaaattggaaacaaggagagaaggaaactacttgtattctgatcttggcttccaggaaacgaggcaacattcttcagctctctctctcgagatatattttctcccttgaaattgtctccttttcccttatgaactggcttttatgaataggccattttctgaagtcatgaatttggacatcacctgcacttgggcatggtggctcacgcctgtaatcccagcactttgggaggccgaggcgggtggatcattgaggtcaggaatttgaaaccagatatcgtctgcacatgatgaaactcactaccatttattgagcatttagtataggaccattttaagatgctgtagtactgtatttgatacaccttatatttaattttcagatcatctctataagttagatattatttaaccatctatagatgaagaaatagactcaaagctacatagctagaaagtaacagagttggaataggatcaagatctttctccttcccaacagtgacctttttataatgcaacatagcctacttatttattcaataaataccattttattgcccagtaataatagtgaacatttattggatgcttactaagtgtcaggcactgtgtccagtgttttatatgtattatcacatttagtcctcatattccattgggctcggtaccgttattatccccattttccaggtgagtaaactgactttccctgaacctgattcatctaatactaaccctgtcaaattaaatcccagggtaaccagccttcagctcgaatcacacattgattctctcatctgtgttaactgtcttttctcctttacagcttctgaatctgttcttattttttgcttgctctaggaaattttataatttctctcactttctaccattattatgatctctattcaggcaaacttgcaatgaaaattctttttccctctgctttttaactctgtcctccattctttcaggctcttgggtaactgttatagaactgagttagaggagacatcttgttttaaccctgtgctgaatgactctcagggcacctgctccctcataccttgagacattgcagctttcccctctttcactggtgaaggtctctgttccctcctgtctgcccatcacattctgacttctttgccattttttagcccagcctctgccttatctacccctgcattctgcaaaagctaccctttcctcccattcatccaggtgctcttccaaactcaaaccttccatcctcttttttgagtacataggggttagaacagaaattttccctgctaatcatgctgactatacaggaaactgatcatgctattcttcacccaccaccttcaggatctgtttgtttctagagtgtgtctcagattgttcattacctgcctcatttgtccagttgtatttgaagggcccttctcttctagttctgcctttttcacctcactgatagacatggcttgaggaatatattaagcatcacattttagatttgaacctaggagaggcctttaagatgtatagagtttactccgtttgttagaaataagctctttccaagactcttcttgacaaccgttgtctacagttttcattgaaaatttcttttaacaaatgttttgaagactcttacatacctgacattgttaacagagatacagcaaggaacaagggctgtagcattggccctcgtgacatttataagaaatggaggaagacattgactatttccttaagaaaaagactacagttaccctttggggtacttgcagtgtacagtgtgacatactaataggtaacctttttgtgtaatcagtaaaggcaaatggatttttaatatcacttagtatattagtggtgatcagtagccagatgtttaaaaaaaaggaattaatttgcaactgttggttttaacaaattaaaggatggatagttttagggaatagtatagtacagacttcttggtgtcttgaatatactcctgaagctgctcaatatttagcgattgttagacttgtgttcttctttttttatggtagattaaacaaatgtagatggttttagaagatgaggaagtggctgatttattcggatagaatctaggaagaaatcaagtcaactttattttgtgacttaattgcctgttaaagtaggctcacatttctgagtagaccagagcagtgctccttagactttaatgtgtaaagtcactgtggaatcttattaaaatgcagattctgatttagtagttctggggtgaggcctgagattctgcatttcttgtaaggttccaggtgatgcttttctgctggtgcatggactctactttgaataacaagggaatggagtattcctaatttgttttgcaatctactgctgtgtactgttatggaaaaagaagaagttttaaacatgcttaagttattaatgtaaaaatcaaatgctagatgtatttagatttaattattttttatcctaacaggaattttgtgtgtcttctggaaccgtattatattgatcagtaactgttgctcattttacctgaatcaaaagggccaatgatttggttaatcttattatcaccttcactttacacatgcgaaaatagtggctctgagtttcaggttcatcatcatggggctagaaaataaatgacagagctagaatttaggtttttaattcaagtcttgtccctctccctgtatcaagctgcctctactccatgtgactccccttgccatcatctaagatgatttcctactgtattcttgtataccttttctttagttcttctaggggtttgggtgttctaaaatttttttaattactgttttttcttttgggagattatagtaaaactttattagcttagcttacttggggaagatagttcattctgactggtggagatcctgatttgcaaaaagctttaaaagaaatcagaaaaaaaattggtctattagaggtttttaaggaagttgctttaataaacatactgataatttgttgtaagtattaattatagttgtttgtgtattaagtagaagttacctgggttagttgaaaactattagattacaggcattttattatttattaaaatctggtttatattgtcatgaaaaggagtttttaaaaatagcctctctaaataattttaaaaatcagagtgagaatttttatgtattttatctcctgaataaataaatacagaattataaaatgtcttctcagtctatgtcataaaatgtttttttattctcagtgtaaactcttctaagtttaattattcatggtaatttttatttaatttggtttaacttttctctttgaagatagaggaggatttagtggttgatcagtgtcaataataatgaaatgccaaaaattattgaactctgacactgtgtcagatgctgtatacatgttgttctattcctcacaacagccctactatcatctataaggccaggggcactaagtaacttcttgggtttattcagctggtaagtgaagagtctaatgctggtaagtctgatttgagagtccatattcagaatatagaactctctaggttgagaagaaaaatcctggaagacattgaggggcttgacttttaaggggaggggctcagattatctctgacatgagacatactttgtgaagatgtaaatttgggagagaataggctgcagcagaagggaatgcatgacgtcgtggtatccatgtgtgctaggaaccttcgcatactttggttcatttagttatcacatcttaaccctatgaagtaactatttcctcattaaaaaaagaaaaaggaagaacagttggttttccagctatagattctgttctgcaccctttccttttggaaatttctcggtctttgtgacatggttttctcttttcctgcctctctggcttatttgctgccatttcacccactcatcttcaactgtcctcagagtcattcctttttcatgttattctctcactgtacatgctccttcccttgtgacctcaaactctggtgtgattgtcattgtcagtgacccctctgtgtgtccacagccctggcagttcctctgcagctccagaggctactggacgcctccacctagctgtcccaaaagtagctcagatgcgagatgttcaacattcacctgatcagtttctcccagatctgctcctcttcctttactctgttatggatcatgacttcactgtctgtgttgccagatagaccagaaatctgggagtaatcctgaacttttctttgactctcttctctcacatccacaacagtcgtggagtctgtagattttacccacgaaaccttcgtatctacctatcctgttcattgccagtgcagctccctaacccaggtttccatcatctcttatgtcctagtggtctccaagtctgcctgcctgtcagaatcacctgggggagctcagatcatagtgtccagtcccacccgcagagattttgattcagtaggacctgggaatctataattttagcaagcacatgattcttacatgggtagtctagtggtagacaagtattgagaaccactgatgcagttggttttcctgcctcttgacatatccccttcccctcagtccacaccccactgcctctaccatgatcttttgaaagccaaaactggctgggtgtggtggctcatgactgtaatcccagcactttgggaggctgaggtaggtgggttgcttgagctcaggggttcagggccagcctgagcaacatggcgaatcctcgtctctacaaaaaacacaaaaattagctgggtgttgtggcatgcacctgtagtccaagctacttgggtggctgaggcacgaaaatcgcttacgccctggaggtggaggctgcagtgagctgagatcatgccactgcattctagtctgggcaacagagccagacccagtcccaaaacaaacaaacaaacaaacaaacaaaaacaatcaaatatctcttcattatctacatgataaattccagcatcctcagtgtagtttgtaagacctcctaggggatcatcacagtcatttcctgccctcttcctttgtatatatcctcttcatcatgtctgcttagtttccagattgttttatttcttgcttctgggctgcttatacattcagttctctcagggtattcagatattctcatttgcttatatcttgtcattgtactaagggaacttttgtaggtctttctaattctgccccgtgaaaaaagacttttctccctgtctgcttgtcaaattcctcttctttgaaaaaaagttcaaatgtctgtgtttataccctgccttctccccttttctgtcctaccactgttcataagacactcctctgacataacactcatcacactgcatagcagttgaattctcatgtctgtctcttcctctgctagttgagcattgagagatcagggaccaagtgatctggccaaaatcactttgctgcaaaggagtagtatatgactttggagtatatgacttaccatctattccaccatgctatgtgatcttaagataaaaatcagattaagaataaaaacttattggtggaagatattcaagggaaaccccatttcaggtggactctattttttttttttgagaaggagtctctctctctctctctgtcacccaggctggagtgcagtgcgtgattccagttcactccaacctccacctcccggattcaagcaattctcctgcctcagcctcccgagtagctgggattacaggcgcacaccaccacgcccagctaaattttttttgtatttttagtagagacagggtttcaccatgttggccaggctggtcttgaactcctgacctcaagtgattcgcctgcctcggcctcccaaagtgctgggattacaggcgtgagccactgcacccggccccctcattttttataaaggaataatacgcaaatagtaaaattgactccttttagtgaacagtttttgaattttgacaaacacatagttacataattattacgtaaccttttaataaagataaaattgtatgattgctgaacacattaaatcagtccctcttggcataagctttaaaaatcagtctcaaaatattttcattacaaactatttattaactctttaagggtccgcaagggttaaggtgtaatgtgccttagtaattctcataattaaaataaaggcttttgctcctgagctcaatttgctatgatatttggctttaggtttgcttaaattttgttcaaaactatcatgggaaacccattaccaaaggagacaactccttctgtgtacaaaagggaaagacccactggttagaaatttcctgaattcaggtgaaatttactttcctataacttctgttggttctaattcctctctctgaaaatggagattaaatctgatttctcataagttctcttagtatttgctgaggcctctttctcttttccaggctttagtctttagtctttttcccttattctgtgtcatggctttgaagcattcatgatcctggcccaagtctgccagatgctgcacctaatgaagtataagacttggctcctttgagtaactcttgtcattctttctattgagggaacttttaaaagtctgtcttattttctttcccttgaaagaatagtcttaaaatatagcatttgtatctataattcagaaaaaaatcgatgggaaattctctcccttaagattaaagtacatggtgattgagtagtgcctacaacagctgctaggttacatgaggctcttgctttacgtgcagtggcaagtctcttgacctctctgaatctcatttttaagataaagggatcatttctaaatctaaaattttatgattctagtttaggtaaaggtttacatgacaacgtgaaaggcttaaaatggttaataggctcaaaataagagcaaatttattaacacatgaaggtacaaacatgttaataagcatattaatagtataaaatataaaaataatttgataaacaaatttgataatttgataaatttgacgagtccatttatatttgtgtcattttttccccctccaccccaccaaaactttctaaagagaaattcccaagaatgctacataaaacatatttggatctttgtactatttggcgacagggtttgacttcagttggtggtaggagaggtaggattttagtcgaagtttttcagtcttttttcttaatgaattaatgacatggaatcccactgataactgtgtaggattaggatgttgacaacataatagaaatgtatctcctgtcacactaatatgtataatctacatttggattcaatacatagcccaaatgagaggatttaatttagctgtaaaatattaatatatatttttaaacattcgagttaatgaaaatatggataatttaatatatttaaagtttacataaaatacatgaagcattagtattcatgattttcctttactgttttagccccccaacctttttttggtttaaaactcaggtggaactgttagtaatgatgaattttcatatcgttgtatggcactattgtgacacagccctttttagtggaaaaggaaacatatgaaagaaatatagaatatttgcatctcatcagaagcactaaaggaaatattaatgcaaatacatgcagaaaaacaaacataactagtatagtattataacatgtaaagacatggaggaatttcatccaaatattattaaaaggttgggaattgttatgctgtggattaggggttgacagttactgtagcttccatgataatcctgggaggctgaatttttgaaagctctctgaccctgctgaactagaagggctaatttgagaagggtatggtgtaaaatgtggccttttataggctgaaaacagggatttgttaagtggttgttgaatgaatgcatgccttgcattgttgggcaaagaagtgagggcattgcttttttcccctggagtaaggaatgaagtaaaaggtcattgagctttgtagagcacatatattcactaaatactaggttgatgagttgttgacttgaggataaagttattatgccagcatagaggaggatctgggaaaagctctaggaagggttcttggacactgaggaaaccttgaatatcaacaaatgtgcttttggatttggcagaaaggatagagaatagaggaatagtgtgaaaaaattatctactctttatttattcatcaaatatatttttaggtactttgataggccttgcaatacaagaatgaaacacccagcccctcttttcagggagaaaacagtttaaaagaaggtcactggacagtgtaatgagtgctgtaatggaatagtaacatgtgcaggtgctctggtcatgcaaaatgcgtcccctgaccagttaaaattttcttgatccagttttcctcaccattagtctattcttcctcctaataattatttttaaaagtaacaattttcagaagttaaaataaacctaaatgatcatgtagtttgttactgctcaaaaggggatggtccatggaataatggcatagcatcagttagagcttcctggaaaatgtggtcttcttagaaatgggctgcatgggctgggcatggtggctcacgcctgtaatcctaacactttgggaggccaaggtggatggattgcttgagtttaggagtttgagaccagcctaggcagcatgacaaaatcctgtctctacaaaaaatacaaaaagtaatagggtgtgttggcgtgtgcctgtaggaccagctacttgggagtctgaggtgggaggatggcttgagctcaggaggctgagcctgcagtggacggagatcgtgctactttactccagcctaggtgacacaacgagactctgtctcaaaaaagaaaaaagaaaagaaaagaaatgggctgcatatctgacccactgtctcaaaatctgcatttttaccatgcccacccgcccccctccccaacctggtgattcgttttctcattgaaactggagaagcactaatctggttcacctacttcccatccagttttacagatggaggaaatgaagatagattgtgctcctaaggtcctatgcacagtagcagagccagtgttagaactcagccagtcagctggtcatggtggcttcttactagtgcctctttccttctctcttttggaaattctctgttgaatgagagaacaacagtgcaccacactatatataaaaaagaatgaacatttttatttacttataatttgtggcatcagcaactcaggaggctgaggcaggtggatcacttgagccctggaggtggaggctgcagtgagccatgtgtgcaccactgccctctagtctggacaatagaatgagactccatttcttaaaaaaaaaaaaaaaaaaaaaaaaaaaacccagtcaccagtctacttactggtagggtcttgtgtgctttctctgatgcctctgttatttcctgtttctctttacttttttactcttctattggcctctattagtgtttcatattgtaagatcttcagcttctcactagtgtctctttccctctctcctgtggtagtcctctctttaaatttctctgttgaatgaaaacgatacagcataccacactgtacatcaaaaagaatgagcacttttacttactcttaatttctggcagaggtttggacctatttctttaattttttttttctacccgctactcagaactttctttgaagtcagtaggaattctgtgtgtggtaagggtgagagataatctatgatgttaggaattaaaatctcctcacagatacatttatcttcctttctttgttacatggagtatggcattaatcagagtacttttgctctttgtataagtctcatgggaaaaacagcgcctgccttaaagttggtggaaaagaacaggataccacagcaactccttctctgtgacctagaacagagagtttgtctaaatgtttcttctctaatatagggggtttgtctaaatctttttttttctataatatagaaaaagaaatacctaagtagatgcacacctcatgttttatagggggcctttagcatagaaagatatggtcatagaatgataatgaacataaaatgataaaatgccattaagaaaagtgaacagtcactgataagaccttcgtggcacaacgcctttttaacaaaagtcagccgtgctatcttttaagtcatttcctccttgtgagaagtttaccatctctcctgtggttcaaatatattggtttatttttaactctgaaatgtgtttgatttgagtgatatagccataatctctatgaatagtagacctccttttaaaactatgaaataaaatcagtttttctgttttcctggttcccatatgtgtttttctttcaagtcattatcacctttttttcccatatctcgagtcatcaagcaatagctcaccttcttaacaactctgatttcattaaaaccatagttgtgtcatttcctgactttgcaaattgtttgattaaatattgtgtttcgtatcttgtatcttactgtctattgacagtatatagacacagattaattttttaagtaaggtattttaatacctagttcactaatccagaagcattttgcatatacaggctttagtaaaaatgacagtttaaatagggagttattatgatcaacaattaagttaaattcatttaggttggttctaagaagttgaattttcttttactactttaaaaatacattatttcttcaaaatacgacataaaatgttttgcattgccaagaataaactagaaattagatttgcaaccttcaactgttgtttagcttttagaaatataaaaattaatattaatatatttttataatgtgttaaaagatgaatttttgttaatcccatccatatttcattatcatgcttgtttcagaaatgttttacttgtggcaacattccattataaaattgatttatggttgtgtatttgaatatgctggacaatcaattcattttaagtaaaataatgtgttgcattttatatatatagttacacatatgtaagttatgcgatttacagctctagagaaaaacattcaagttagttattcatgggaataaaataggtgcatgttctcaatgaaaaagttagaaaaatgtcttgaatatggtgactgtttacattatagaattgtaattaaccctaacttaatctctcagtgcccctgtactcttgtcttcagtgtctcgagatatttcagcaaatggctaccatcgccccgtacgtacctctccagttacattcttgcctgctttaggagtggacacacagagcagcctccaagctgccctgataaatttcacgtgtataatatggaagacaaatgtttccttaacatatttattttctgtgtatgaagtttttcattaacttttcatcccaagataaccacctctgtttttagtgcgctttatttcttgtttttgttctgtagggtctctgtaaaacataatgtgtgacataagtaatgaagcataattttcaaaagccacttgcaggagtcagtattgtttctttggtttttataccatgtattttttgttgggactcaaaggacagtgatccgtatttagtcaaattaggaaattaagttgaaacatcttgattcctaaaaagtgtattttataaaacatttactgattaatgaattttatggtattttgttctctctatagataagcgtgtggaaaattggcctctgatgcagtctccttggcctacactaagtataagcactctttatctcctgtttgtgtggctgggtccaaaatggatgaaggaccgagaaccttttcagatgcgtctagtgctcattatctataattttgggatggttttgcttaacctctttatcttcagagaggtatgtttttaagatcactttaataattttccaaggttattggaaatttaaaaatgagaatgtgtaaaaccatcattgtaatattttacccagacataactgttagctactctaaaagtacatttttgacatttttcacttttgaataacaaaaatattaaacatgaggtataggaaagttgtttaaagtggaattttgaataagaccaactggattctaaagtaacccttattagctgttctggcattgaaagttatttaacttttcttagcctcgttttccccacttaaatacctaccatctcaggttattatcggagtaaagtaagataatttttgcatagtgtttggcacgtattaagcactcagtaaacagtagcagctatttatttataattcttattcagtagtttctggtttggtaactgatcaaaatatgccaatttcaagtaaatcctattacataatgttgtattttgggctggaattaaaatgaattaactgccatatagctgttgttactaatcattatatacttttctgggcactagcaaattctccacaacattttcaaaggaaatcctaaaggctagattgacacagccattttttaaattgcatatgtataaagaatttgtttcctaagagactcattaaaattctgtatgaagccagagaactgactagtaatgctaggaaaataaacaataactcttcttagaaaaagaaatatttctttacttgaaaacttagcctcgagtctcaacttcatgaacactttaatttatttggatacctgtacctaatttgttagatgcaaaccactctggatcaaacatagaaaccagttaaggtaggacaactttgaaatctgagtcctagcaataaaaatttctcttaatactgtgaagtatttaatagatagagtcaatgttcagtataaaatttacatgtgatttttaatttagattttaattatagaacaaattaatgcacagtaacttctagcaatcggaatgcatgaaatttttaatgcatttgaaatttttaaagaaaatattgtgtttaaaataatttgaaaggctacattttgtatataattgtgtttttaatgctgtgtttactaaaactttactacaaatattattactctttttccagttattcatgggatcatataatgcgggatatagctatatttgccagagtgtggattattctaataatgttcatgaagtcagggtaagtacattaaaaatactcttaatcagtaaaagtggtttgatttttataggccccagtctgtgaaaatatagaatgtacttttttttaagagacagtttctttaacattcatttagagacagtttcttgctctgtcccccagactgaagtgcagtggtatgatcatagctcactgcagcctcaaactcctgggctcaattgatccttctgcctcagcctcccaagtagctgggactacgggtgtaccccaccatgcctagatatttttttactttttgtagagacagggtcttgctatgttgcccagtctggtcttgaactcccagccgcaagggatcctcttgcctcagcctcccaaagtgctgagattacaggtgtgagccaccacacctggcaaaatgtactttaatatgatagtgaaattatactagattgttatgagtcagtaaaactatgcttttttaaaaagtacttaatttaaaatttgtttttgtatctcaaaaatgttttttattttgcagcatgagtgaatatgcattgaattactagaatgcagtacttgttaaagcaaagaagtctcatgtttattttaaagacattattttagatttttgtttgtttgtttttgctcaagagtagaaactcttagagtccaagttatgtgttaagaaagtgaggaaaagtaaatgttttgtgaagtgtggaatatttaggaaattaggcatagaacaatttgtattggcataaactcagtttttttatatttgcagaaaaataaaacactagtaacaagatgttatattttattttccattcacttacatgagtaaaattaacattaagcaaaagaaacaacagattaccttattctattacgtatttattgaactcttcttatatactgtggttcatactagaaattacatataaaacataacatgttttatgttacctatagtatatgtagtaataatatctagtggcttcccttgagaaaaatagtttcctggcatggggagtggacaatatataaatatcagtattcatcaagactggagacaaaattggtttaatgtacttaacaattttatgtacgtatcttcatttccctattagaagacttatggactgatgaaaaatcacctgtcttggttgtcatgttggttttatttattcattaaatatgtattgagcatctactatatgttggtactggggacaggggtgaaaagccctgcctttgtggggctggcattttagtgagggtgagaaggcagacagcagccatgtaaacaaatgactaaggaggaaataaaacctagaaggtgactgagagaaaatgagagactactcagatgtattggctatgaaaaattttcttctgataactattgccaaagaatctcttcagttggagggctgacttcttgcttgccgtagggaagggaacagggataagtgtttgactgactgacttctgggagtatgcatagaaaaacccttgggtggggcaacatattactatttttaggataaaggtttaaaaataccactctcacttacagatgtaaattttcaagtctttaaaaaatgagctattatgagaactgtaatctaggaggataaagaacctgagatttgcaacagatttcatttgaaatcttcctcgtgctctcttctcctctctaatatgggatgcaatagagtgaatattttacgcagagactcatttctgtattcattgttacgtagccaagaagtatcactttttggtgtagtttatttaaagatatctagtgcagtttgcaaactaagcagacttatttgttgttattttatgtgaattgtggcaaatgtagaggtccagatagaggagaaataagagattgtatctttccttctagttaaaggttacgatgccaaaatggtgacagttactttattttaaagaactaatataatagtgaatattaactatagtgttgctgggagtagttaactatttcaagcttattttcaaccaattcatatcatttttaatgatatttttcaagtaaattcagtaaataattctcagtttgtaaatactttgtttcttatatgctttctgtttcttctttccccatactttaaaattataattttgtttttcaaatgcaaaagccaaaacaaacaaaaatctcatgacatataccaggtgcaggtataaaatgcaaagggatccttcaactctaaaacacctaagcctgtctgtgaatgaatctgacagggccctaacacatgaaagcttataaatagattttagttatgtactgccttaattgttatttgcacataggatgcttatcatagaaaatacttgacataaaaaattaagtttggtttactgttacagtaggattcttgtaaaacattttaaaacatacagaggtcacctctcactggatccttaatctcagtatgtaatgtaatctttctgtaccatctggtgccttaattagagcccaggaaaagaaaagacttattcatgtaagcaggattatatttatgacctgggaaatatttgtttgttcattccaattttttattttaaagatttaatacctaatacaaactgctgtgattggatatttatgttgttgcacatattggtgcagtcggagaggtgagggagagagggaggtgaggggagagggtgggcagacaggcagacacttgtagagaccagttgctacatgaacttacttgcaagtttttctgaggtagtataatgtaggctaagcatgaacaactaaaatcattttacaaagtaaacatcttagttcctctaccaagttgaaaagcacagcagtctgaatgtgcttccagctctgtattcagcagtttgacactatgtaacatctacctatggaatagacagaaaatgttctctaacttattggcactgtatagatagagctttggactctttcttttctatttttccccatggagagatgcttaggtttttcaaacctacttagccattaatgattccatgccttgtacattttgtgcaatatacaaatgtttattttggacttacttacaatgagtataaacccatacaatagtgtcattttggtgtatataacacgctttccctttttacagatagctgctgctctgtggtggtactttgtatctaaaggagttgagtatttggacacagtgttttttattctgagaaagaaaaacaaccaagtttctttccttcatgtgtatcatcactgtacgatgtttaccttgtggtggattggaattaagtgggttgcaggaggacaaggtgagcattttcaggaatatactgcttgcgtttaattgcatatatgtgttcagtggaaagcaatgagaacctaggactttgacttgatctaccatttaacttgctttcatggttaatcatttccatgttcatttcttttttttttttttttttttttttttttttgagatggagtctcgctctgtcaccaggctggagtgcagtggcgcgatctcggctcactgcaacctccacctcccgggttccagcgattctcctgcctcagcctcctgagtagctgggactacaggcacacaccaccacgcctagctaattttttgtatttttagtagagacagggtttcaccatgttggccaggatggtctcgatctcttgaccttgtgatccgcctgcctcggccttccaaagtgttgggattacaagcatgagccaccacacccagccatcatttcattttttaaaaaatttgaggcaagtataacttttgctttttatctgtgactactttaatacctaaagatttgccccagagatgagcataaaccaagactaaaggtagaaaacaatattttgatggggaacgggtctagtagttctgatccaaagaataccaagacacagcccctcaaatctgaagatttccatcttagcccatagctcactcctgccctaatacagattgaacatccctaatctaaacatctgaaatccaaatgctccaaaattcaaaactttttgagcaccagcatgataccacaggtggaaaattacacacctgacctcatgtgatgggtcacagtcaaaacacaagtgcagaacacactttattcagtatacctaagggaaaaaagaccctcttagcccttttccatgtacagcataatgttgatgccaaacaaccacagataatgtccacacgggtgacatctttgctttctgatgttcagagtacacaaactttgtttaatgcataaaattattttaaaattttataaaattaccttcaggatacatgtataaggtgtatatgaagttttggatttagacctgagtcctatccccaagacacatcattatgtctatgcaaatattccaaagtttaaaaaaatctgaaatcagaaacacatctggtcctgagcattttggataaggaatactcagcctgtataggtcttgttggttcttattcaaaggaaattttatatagccttgttaatgttaaagaggatctaaattcatagcttggccaagatctgcctcctgtcttttcagatgcctgtagattaaggatgattcacattgttgagcttgatgcagattattcctctgaggaggatccagggtacaagtcattatagtatagttcaccttgctgctttccattgtcatattatgataattggcagcttaggaattcatacatatattattgggggcccagtttaaaacaatacagttaaaggcaacttctgtaattttaaaagaaaattagtaaatctgaactgacttagatcactttatgacaaaaattaattattattacatctataccttgattaaattaatattctgaaaagaaagaatgtcattgagtttgactgactggaatatctctgtagatttatctatattaaatgaattattcaatgtgtgtagtgctgtcatattttaaactatttagaaacctaagaaataagtactgttttgttgcagatatatatattggcacatctaaaggcaagaatagtagaattaaaagtaacataggcaacagttctaagggcctatttaagctcactggagtactccatttgaaaacctacaactgtgaaagtcctttgcatttgaacatacatttttatttatatggtaaaacctcaaatctaaactttaaaaatatctagcttaatctgaagggaaaactttttcattagtactactttatatttttcatttctaatctgtactcaagattaaatattaaaaaataatgtgtattcatttattgtacatctcagtggcttactgcctaataaaattttctgtatcttgtaattacctgttgtttttctaaagcattttttggagcccagttgaattcctttatccatgtgattatgtactcatactatgggttaactgcatttggcccatggattcagaaatatctttggtggaaacgatacctgactatgttgcaactggtgagttaaatgcttccaaagtttcttctggtaaaatactgaaattgtttaaatttgattaattttaaagtgcaatgtcattttagacaattttctattatgaaattaactttatcctccagctatatcgcagttatgcccacttgactctagatgacctattcatagacttattggtcccagcaaatctttgtttttataatatgcttaaaagactagacatagcccttactcctctaaggaaaacattctctaaagatgtttgtggaaaggatgattaaagatctaagcacgctacaaagtacgtagtatgaatagaaaaataatatgtattttttccctacaggtgattttcttaacttaaaattttattacctttcttcatcttgtccttgcttaaaaattttgttttttatttcatttttcagtctttaacatatctcatatatttagactaaacagctgaaaagaaagcacatagccaaggaaggggtggggcaaaggaagggaataagggcaagagaactccttgtaattctctaaaacactgtccttaagagagcagagacagatattttcaaatggtggtctttagtccagacagttttcagcacatcccgctagtaagactgtttgtggcaatctgagtgtgtgcattagtgtacctttcagatgctaggcactagcctgtaatcaaactggaaattcaaagccaacctggtaactatttttcaagctttagaatttattataatttgctttaatcatagcatgtgcacaattttatcagtggaatcacatttggaaataatattcatctttgctttaagttgctaaacagctggctaaccaaacaggaagcacaacttttgggaaatgctgggtgcgaaggattcatttgtaaccggagtggtataggaagcttcctccagtttttgtgtaaaagctgttaacacagtttgtgtaattcatttctctcagtaagtagtaaatggacctaagaaaatgaagaggtgatcaaaatcatgtgaattagtcctatctggtttgttggagcctacacctggctctccttgagtgatgccatacccaatcacacccttatacacaccctttttggtgatgcagcttctaaaaaccattgtgaaaccacaagttcttcaaattaaaatatttgccaaggaaaattataaaagtttaatatagtataaatttttagcttacatggcatacattattttaatatttaaaataagtaatactgattattttaatcaataattaatattttaagaacaatgctatccttaaaagagaaaccacaattgtccatttcaaacaggatgttgagtttgtatttccaagccagaagttcccacaacagttagtcatataagttaattgtataaaaagttaacccttgttaacattatgtcttgacccattcttttattgatacatcagaatctatttcattttaatcatctcactttgggcttttttccttttaaaaattcttaaagttgaaagtgattctttttgtatttattctgaaatatctactaaattttatccaaatattatttttttctttttttgggacggagttttgctcttgttgcccaagttggagtgcaatggtgcagtctgggctcactgtaacttccgcctcctgggttcaagctattcttctgcttcagcctcctgagtagctgggattacagtcatggaccaccacacctggctaattttttgtatttttagtagaaatggggttttaccatgttggccaggctggtcttgaactcctgacctcagttgatccacccgcctcggcctcccagagtgctgggattgcaggcatgagccatcgcacctggccatcaaaatattagtttttaaaggatagtgttcgttcaaataaaaatttacctgcctactatctgtaagaaataaatgagccattttgataagctcattttaagtatgtgtatctgcttgctgacaatttgatagaaagaaaccaatgtaaaatgtgggaggaaaacaaaattgaagagcctacaatactattactttactctcctaggccattgattccctttcatcagataacattattcaacacatattcattgatcctgatgctgtgctgaggactcaagaatcaccgatgggcaagacaaagccctgccttctagagtcattttctagtggaaagataggcaataaagaaataacaaatacagaagacaatttcaaataggtataattctccttaaaaaaaatgcagaaagctctttggttaatgggattgggagggccaattgaaattgaagtcttgtcagagagtccttactgaggagatggcatttgagttgtgaatgaggagctagccatgggagccagaaaacaataaattttgcacagtgtggcaaaatttgggcctgtgatagctatgtagaagatgccgatgttgttaaaagttgtttactattcagattaaatgttttgtgctgtcatttctgtttttcagattcaattccatgtgaccattgggcacacggcactgtctctttacactgactgccccttccccaaatggatgcactgggctctaattgcctatgcaatcagcttcatatttctctttcttaacttctacattcggacatacaaagagcctaagaaaccaaaagctggaaaaacagccatgaatggtatttcagcaaatggtgtgagcaaatcagaaaaacaactcatgatagaaaatggaaaaaagcagaaaaatggaaaagcaaaaggagattaaattgaactgggccttaactgttgttgacagtgaggaaaaactcccatatcatataaaatttcagggaaaacagaagcaaaggagagcttgggggtggggagaaaagacaaatgtgctctatgtcctagtaactcttagactgagtaaagtgttaataccatacccagatgttttatttatgaagtttttattttaaacattttttttaaaaattagccttgatattctccagaccaaagcaatcattaagtgactttggggattctccccctgttcacatccagttgtctaaaggatgagatttttcatgtatcttatagtcactcattcttcggtctgaattttagacgatcacagaaacggtctttatgaattattttgataaattactaattatcttatctactgactgaaatcagtggtgttacattttcttgtccaaagctgaaaatgtgtatacacttaaacttgcacatttgaattcatttgctgaccggaatggtcaaatctctccacctctagtcagagtataattttggttgtaattaaatttttaaaatctgctgatctctgtagaatcttagaggcttgatgatgatggtgttggtgaaaataagaaagaattgcagtaaagtcttgtctggtgacccagagatcaccatgacttgaggcacaaatcactgtggggaaacaattttttgtgatgaaaaggcagcatttgaatactcctgttagtagcagaaatatattatgaaaattaagattattgtctgattgaaacatgaaacaactcatgtctttattagtaacatcataagatagttacatttatgtgctgttagaatatgttgatttttatcaggctttccttgttttgatttatggctgttcctgatttttcatatgtggaaatatacctacctcttccgttggaaagaacatttaaaattaaataaattttaattaaaaaatcaaggagtcttctaatgtaaattttaatgttaactttcaaatccactagtattttttttgcttttatgacaaatagcatacaccaaacatttctgtgaaactatccttctctttcaatgtgtttaattttggagtaacgttttccttgtgactaagttgcaagatcttatttattaactaggtatgaagtataaacccattttggtgcaatattcttgactccttggtgctaaagattgttaaattcaatgcttgatgttacaaggtgttgttaaaacacaaaatgaataaaagtgagagtagtcagaactataacattcaatttgctatttacaaatgaagtatttcatgtaatataagtgaacaactggaaataaagtaggaaagaatttgtatcatgttttactacataggttaattttttaagggatgttgcaaagggattactagagaaagacaaaatgtgaccaaaaaaaagcatgaatatttcttaagtatctcaacaacatgtcaaagctgcatgtgtaggatgtatgctgtttgtacaaactatttcagaatattttgtaagctataacatatttattgtgcattaaaattaaatactttttccccaaaggcatgcagtcatgagaattacagaaaatttgcaacatataaagtagtttgatctaagaggattcaacacctttgttttgttgctcagtgtgtaatgactgagatttgtaaatctttgtgaacattctgtactggttcccaagagctattcattccctgctacctgatttcagcacaataaatatacttctgctgtgggaaaaatatgatttttttttgttaatcagtttattataatgtaaactgtctgctagatgtttatttgatttagttaattctaaattgttttctgctgctaatttcttatatagaaaaaatcaaactagaacttgggtatctaccatttctggtaaaaagcttattttctatagtagcatatttttactttaaaaattagctgcttttggaagagcttttcattcagacataaaacataccattttcttctaaattattcttaacataaatggggaagttcttgtattctttcatttaaatcattcagtccttactataagtttctttatagaagatgattataagatgtcataaatacagccttttccagtagctttcataatttctctattatcacagatatgttgcatgccttatatctagaaactgatgaataaagattgtgtctcctacttggtaaaactttgtcactaactaaagtaacattttcttacatagaagacaaggaatttgttaaatacttacaataactagaaatacctgagacatgtgagttttcctctataatagatgaccatagctctttctctttcctttgttacttcttcattttttggtagaaatgagatttcactatgttgcccaggctggtcttgaactcctcaccttaagcaatcctcctgccttggcctcccaagtctgttacttttgactgttttgctgatggaaactaacaaatgactagttttagttttaacattattaattcattctctttctcttgttgtgcatgctctcaaggtattcaaaaatattttaacatggattattatgtttgtactttagaacatggaatctagcagtgcttttagtttattaggaatattttaatttacaggcttgtttcatatatgcaactcagctaccctgtaaatgggaaagagaacatagaggtataagtgatttttgttgtattatctatttgacaatacaagtcagatactatatattgactattggagcagaacttttgaatctctctcaccttcagaatgagacaccacactgaagagttcacactctcccagttctttccttgttatagccataatctcagccagctgtactcataacaccgtgcttactaagaaaaaaacagctacagctaaccaggatgtgtgctggctggcctgctcagcatggacctgggtggcagatttcagcatcctgccagatggtaaatagggcagcagaggtgagagactgatactgagctctgtacacaggcacacaacacacaacatctgagcagtaatcataaaacctgctgaaggaaattggtcacctgccactcagcgtgcaagtaattttggcaagtgaccttagcaagtccttattagtggtgtaatttagaatccatgcttagtcttttgtattttaggcagataacttctgaatgacaagaatatttgtgaaaaggaagagaagtagaaatggattccttttttgttgtatttttgcaccaacctaatatttttgtttcaaaatactacaaatcatgtaagtgttagaagttagttttcctataaattacagaggggaaggcacagctgtgaattgaaaggcaaagatccttttgaatttgttttcataggaggctgtgaatcctggaggagattaaaaaacctaaatttaaaataattgatatatagaaccactgccacatgaagattgtaagattttagaatgtatcactggaagagccttgtgacttagccagaaggggatccagagctgtctttagatgatcaatgggaatgctatgagtcagtcaccccttggtgttctttcagacttagaaatgtcggttttgctgtttcctttgagaaattaaactttcttctatctttttcttttaattagggtgttgtttttttccttttccttttttttcttgtctttttcaatttaccacacaaagcatgtttacacatcaatacacatgagc

SEQ ID NO: 2 - DNA, 3085 bp, Homo sapiens
acgtgacgccggctgaggagattggaggggcggctgcgcgaggctgcagactggtgcagcgcactgtgctggcggctgggcctcctccacctcctcgtctttctcccgggaaccttgacgacgccttccgcttggccctgccttctgccgcatccccgccgccgcggcgccttgaggagcaggagaagacgcagccgggccgccgccgttagaggggttcccggccgccgctcgccccgtcggccgccaccgcctccggggtcagccctctctctgggtctccgctttctcctgccgccagcgcccgctcatcgccgcgatggggctcctggactcggagccgggtagtgtcctaaacgtagtgtccacggcactcaacgacacggtagagttctaccgctggacctggtccatcgcagataagcgtgtggaaaattggcctctgatgcagtctccttggcctacactaagtataagcactctttatctcctgtttgtgtggctgggtccaaaatggatgaaggaccgagaaccttttcagatgcgtctagtgctcattatctataattttgggatggttttgcttaacctctttatcttcagagagttattcatgggatcatataatgcgggatatagctatatttgccagagtgtggattattctaataatgttcatgaagtcaggatagctgctgctctgtggtggtactttgtatctaaaggagttgagtatttggacacagtgttttttattctgagaaagaaaaacaaccaagtttctttccttcatgtgtatcatcactgtacgatgtttaccttgtggtggattggaattaagtgggttgcaggaggacaagcattttttggagcccagttgaattcctttatccatgtgattatgtactcatactatgggttaactgcatttggcccatggattcagaaatatctttggtggaaacgatacctgactatgttgcaactgattcaattccatgtgaccattgggcacacggcactgtctctttacactgactgccccttccccaaatggatgcactgggctctaattgcctatgcaatcagcttcatatttctctttcttaacttctacattcggacatacaaagagcctaagaaaccaaaagctggaaaaacagccatgaatggtatttcagcaaatggtgtgagcaaatcagaaaaacaactcatgatagaaaatggaaaaaagcagaaaaatggaaaagcaaaaggagattaaattgaactgggccttaactgttgttgacagtgaggaaaaactcccatatcatataaaatttcagggaaaacagaagcaaaggagagcttgggggtggggagaaaagacaaatgtgctctatgtcctagtaactcttagactgagtaaagtgttaataccatacccagatgttttatttatgaagtttttattttaaacattttttttaaaaattagccttgatattctccagaccaaagcaatcattaagtgactttggggattctccccctgttcacatccagttgtctaaaggatgagatttttcatgtatcttatagtcactcattcttcggtctgaattttagacgatcacagaaacggtctttatgaattattttgataaattactaattatcttatctactgactgaaatcagtggtgttacattttcttgtccaaagctgaaaatgtgtatacacttaaacttgcacatttgaattcatttgctgaccggaatggtcaaatctctccacctctagtcagagtataattttggttgtaattaaatttttaaaatctgctgatctctgtagaatcttagaggcttgatgatgatggtgttggtgaaaataagaaagaattgcagtaaagtcttgtctggtgacccagagatcaccatgacttgaggcacaaatcactgtggggaaacaattttttgtgatgaaaaggcagcatttgaatactcctgttagtagcagaaatatattatgaaaattaagattattgtctgattgaaacatgaaacaactcatgtctttattagtaacatcataagatagttacatttatgtgctgttagaatatgttgatttttatcaggctttccttgttttgatttatggctgttcctgatttttcatatgtggaaatatacctacctcttccgttggaaagaacatttaaaattaaataaattttaattaaaaaatcaaggagtcttctaatgtaaattttaatgttaactttcaaatccactagtattttttttgcttttatgacaaatagcatacaccaaacatttctgtgaaactatccttctctttcaatgtgtttaattttggagtaacgttttccttgtgactaagttgcaagatcttatttattaactaggtatgaagtataaacccattttggtgcaatattcttgactccttggtgctaaagattgttaaattcaatgcttgatgttacaaggtgttgttaaaacacaaaatgaataaaagtgagagtagtcagaactataacattcaatttgctatttacaaatgaagtatttcatgtaatataagtgaacaactggaaataaagtaggaaagaatttgtatcatgttttactacataggttaattttttaagggatgttgcaaagggattactagagaaagacaaaatgtgaccaaaaaaaagcatgaatatttcttaagtatctcaacaacatgtcaaagctgcatgtgtaggatgtatgctgtttgtacaaactatttcagaatattttgtaagctataacatatttattgtgcattaaaattaaatactttttccccaaaggcatgcagtcatgagaattacagaaaatttgcaacatataaagtagtttgatctaagaggattcaacacctttgttttgttgctcagtgtgtaatgactgagatttgtaaatctttgtgaacattctgtactggttcccaagagctattcattccctgctacctgatttcagcacaataaatatacttctgctgtgggaaaaataaaaaaaaaaaaaaaaaaaaaaaaaa

SEQ ID NO: 3 - protein, 314 aa, Homo sapiens
MGLLDSEPGSVLNVVSTALNDTVEFYRWTWSIADKRVENWPLMQSPWPTLSISTLYLLFVWLGPKWMKDREPFQMRLVLIIYNFGMVLLNLFIFRELFMGSYNAGYSYICQSVDYSNNVHEVRIAAALWWYFVSKGVEYLDTVFFILRKKNNQVSFLHVYHHCTMFTLWWIGIKWVAGGQAFFGAQLNSFIHVIMYSYYGLTAFGPWIQKYLWWKRYLTMLQLIQFHVTIGHTALSLYTDCPFPKWMHWALIAYAISFIFLFLNFYIRTYKEPKKPKAGKTAMNGISANGVSKSEKQLMIENGKKQKNGKAKGD